(19) United States
(12) Patent Application Publication
(10) Pub. No.: US 2026/0327332 A1
(43) Pub. Date: Oct. 22, 2026

(54) MEANS AND METHODS FOR THE EXPANSION OF NK-CELLS

(71) Applicant: Christian-Albrechts-Universität zu Kiel, Kiel (DE)

(72) Inventors: Ammelie Svea BOJE, Kiel (DE); Carina Lynn GEHLERT, Kiel (DE); Katarina Julia DIEMER, Kiel (DE); Anna-Kathrin LANGNER, Kiel (DE); Steffen KROHN, Kiel (DE); Katja KLAUSZ, Kiel (DE); Matthias PEIPP, Kiel (DE); Martin GRAMATZKI, Kiel (DE); Christian KELLNER, Kiel (DE)

(73) Assignee: Christian-Albrechts-Universität zu Kiel, Kiel (DE)

(21) Appl. No.: 19/483,770

(22) PCT Filed: May 10, 2024
(86) PCT No.: PCT/EP2024/062898

§ 371 Date: Nov. 12, 2025

(30) Foreign Application Priority Data
May 12, 2023 (EP) 23173193.6

(51) Int. Cl. C07K 16/30; A61K 31/711; A61K 35/00; A61K 38/00; C07K 14/54; C07K 14/715; C12N 5/0783; C12N 15/63

(52) U.S. Cl. CPC C07K 16/30; A61K 31/711; A61K 35/00; A61K 38/00; C07K 14/5443; C07K 14/7155; C12N 5/0636; C12N 5/0646; C12N 15/63; C07K 2317/14; C07K 2317/55; C07K 2317/622; C07K 2319/21; C07K 2319/41; C12N 2501/18; C12N 2501/2312; C12N 2501/2315; C12N 2502/1107; C12N 2502/30

(57) ABSTRACT

The present invention relates to a fusion protein comprising (a) an antibody or an antibody fragment binding to an antigen being expressed on the surface of a target cell of NK cells, T cells and/or NKT cells, preferably the surface of a B cell or a tumor cell, (b) an antibody or an antibody fragment binding to 4-1BB, NKG2D, NKp30, NKp46, NKp44, 2B4, CD28 or DNAMI, and (c) IL-15, IL-2, IL-18, IL-21 or IL-12.

The present invention relates to a fusion protein comprising (a) an antibody or an antibody fragment binding to an antigen being expressed on the surface of a target cell of NK cells, T cells and/or NKT cells, preferably the surface of a B cell or a tumor cell, (b) an antibody or an antibody fragment binding to 4-1BB, NKG2D, NKp30, NKp46, NKp44, CD28, DNAM1 or 2B4, and (c) IL-15, IL-2, IL-21; IL-18 or IL-12.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

More than 40 years ago, in 1975, natural killer (NK) cells were described for the first time. At that point of time they were described on a functional basis as lymphocytes with the ability to recognize and kill tumor cells without previous activation (Herberman, Nunn, Holden, & Lavrin, 1975; Kiessling, Klein, & Wigzell, 1975). During the past two decades there has been a major progress in the understanding of NK cell biology and function (Caligiuri, 2019). NK cells are described as large granular lymphocytes that represent around 10% of the cells of the peripheral blood mononuclear cells (PBMCs). With their ability to detect and directly destroy malignant or infected cells, NK cells form an important part of the first line defense of the innate immune system (J. Wagner, Pfannenstiel, & Waldmann, 2017). The NK cell cytotoxic response against malignant or infected cells is direct and does not require prior antigen-priming. Rather is the detection of malignant or infected cells controlled by a set of activating and inhibiting receptors that recognize the absence of self-proteins or presence of stress ligands on target cells (P. S. A. Becker et al., 2016). The interplay between activating and inhibiting factors controls the reaction of NK cells. The outcome defines whether the NK cells were activated or not. When the inhibitory receptors interact with HLA ligands, the receptors transmit negative signals that lead to an inhibition of the NK mediated response. In contrast, in the absence of this kind of interactions as it may take place in virally infected target or tumor cells, the activating receptors transmit triggering signals to the NK cells. This results in target cell killing (Bottino et al., 2003). The preponderance of activating signals is another way of triggering the NK cells into target cell killing. The NK cell inhibition is substantially mediated by the CD94/NKG2A heterodimer as well as the polymorphic inhibitory killer cell immunoglobulin-like receptors (KIRs). The task of the NK cells here is to differentiate between self- and missing-self. This is important to prevent their target cell killing on healthy self-tissues (Bottino et al., 2003). Both receptor types interact with their corresponding human-leukocyte-antigen (HLA) that are molecules of the major histocompatibility complex (MHC) (P. S. A. Becker et al., 2016; Moretta et al., 1996; Pérez-Villar et al., 1997). The KIRs can be distinguished into KIR2D, KIR2DL1, KIR2DL2, KIR2DS4, KIR3DL1 and others. They bind the HLA-ligands by using amino acid (AA) structures in the alpha-1 helix of the HLA molecule. The HLA-ligands are classified into three subgroups: HLA-group 1 (C1), HLA-group 2 (C2) and HLA-Bw4. All three subgroups bind to the KIRs by a long extracellular immunoglobulin domain (P. S. A. Becker et al., 2016). Well-characterized activating receptors are the natural cytotoxicity receptors (NCRs) NKp30 (CD337), NKp44 (CD336) and NKp46 (CD335), which recognize specific ligands on virally infected or tumor cells. Besides the typical bacteria or viral structures like haemagglutinin A (HA), NKp30 is able to interact with a member of the B7 family of immunoreceptors called B7-H6 as well as the HLA-B associated transcript 3 called BAT-3 (Brandt et al., 2009; Strandmann et al., 2007). While NKp30 as well as NKp46 are present on all NK cells, NKp44 represents a receptor that is specifically expressed on activated NK cells (Vitale et al., 1998). Besides the NCRs, the C-type lectin like receptor NKG2D (CD324) as well as the DNAX accessory molecule-1 (DNAM-1; CD226) portrait two other NK cell activating receptors (Andrade et al., 2015; P. S. A. Becker et al., 2016). The DNAM-1 molecule interacts with poliovirus receptor (PVR; CD155) as well as the human plasma membrane glycoprotein nectin-2 (CD112) (Bottino et al., 2003). NKG2D interacts with the unique long 16-binding proteins 1-6 (ULBP 1-6) as well as MHC class I polypeptide-related sequence A/B ligands (MIC A/B) (Lanier, 2015). The signal transduction of the activating signal occurs via transmembrane adaptor proteins that contain immunoreceptor tyrosine-based activation motifs (ITAMs). These ITAMs activate different signal pathways by phosphorylation. While NKp30 related signals are transmitted via the CD32, NKp46 signaling is redirected via the CD34 chain as well as the Fc-receptor γ-chain (FcRγ). Whereas NKp44 signal transduction is mediated via the DNAX activating protein of 12 kDa (DAP12), NKG2D signals through DAP10. In vivo as well as ex vivo development and survival of NK cells require cytokines like IL-2, IL-4, IL-9, IL-15, IL-18 and IL-21 (Fujisaki et al., 2009; Miller et al., 2005). Besides the described receptor molecules, the NK cell activation can also be triggered by these cytokines. Moreover, it could be shown that IL-2 and IL-15 have similar impacts on NK cell development and survival (Giril et al., 1994). In addition, NK cells can be activated by the binding of IgG antibodies on the antigen CD16a (FcγRIIIa). The binding to CD16a could be shown to induce antibody-dependent cell-mediated cytotoxicity (ADCC) leading to an NK cell-mediated killing of antibody-coated target cells (Bruenke et al., 2004). Phenotypically, NK cells are described as CD56+ while being CD3− (Freud et al., 2005). Moreover, human NK cells can be roughly distinguished into two subpopulations. The partition underlies their CD56 cell surface expression. A distinction is made between CD56dim NK cells that show a low CD56 expression and CD56bright NK cells that show an increased CD56 expression. At the same time CD56dim NK cells show higher expression levels of the Fc-receptor FcγRIIIa (CD16a) compared to the CD56bright NK cells. These cells are CD16a-negative/-low (Freud et al., 2005). Here, CD56dim NK cells show an increased ADCC compared to CD56bright NK cells due to their CD16a expression. When NK cells are activated, an immunological synapse/lytic synapse is formed. The formation can be split into three steps: The initiation step is when an interaction between the NK cell and the target cell is built. The effector step is when NK cell secretes vesicles with lytic granules that contain granzymes and perforins into the cell-cell interface and therefore into the target cell. And lastly, there is the termination step when the target cell is lysed. During the last step, the perforins destruct the cell membrane of the target cell and the granzymes lyse the target cell (Orange, 2008). Alongside the perforins and granzymes, NK cells are able to lyse target cells by the binding to the Tumor Necrosis Factor Related Apoptosis Inducing Ligand (TRAIL) (Smyth et al., 2005). Likewise, NK cells produce a variety of immunostimulatory cytokines and chemokines like the tumor necrosis factor-α (TNF-α) and interferon-γ (IFN-γ). Beside the immunosurveillance, NK cells are able to interact with other immune cells via cytokines like interleukin 3 (IL-3) or 10 (IL-10) (Vivier et al., 2011).

The basics of NK cells in cancer therapy were laid in 1980 and 1984 by two studies that found that NK cells effectively eliminate tumor cells in mice without prior stimulation (Mice et al., 1984; Riccardi, Santoni, & Barlozzari, 1980). It is known that NK cells have the ability to recognize and destroy tumor cells due to molecules of their MHC. Only one year later, it could be demonstrated that NK cells spontaneously kill MHC class-I-deficient tumor cells in vivo as well as in vitro (Ljunggren & Karre, 1985). Although NK cell-based immunotherapy for cancer represents a great promise, it was observed that tumors develop various strategies to escape the NK cell attack or to impair the function and activity of NK cells (Hu, Tian, & Zhang, 2019). Even under normal conditions NK cells represent only a small portion of peripheral blood mononuclear cells (P. S. A. Becker et al., 2016). It could be recognized that patients that suffer from various hematologic malignancies and solid tumors show lower NK cell numbers as well as NK cell dysfunction (Fujisaki et al., 2009). And it could be observed that a low NK cell number as well as a NK cell dysfunction correlates with a poor prognosis among tumor patients. It was also found that, under the settings of tumors and chronic infections, NK cells can show an exhausted phenotype. Such a phenotype is normally characterized by decreased effector functions as well as altered phenotypes (Gardiner, 2017). Decreased effector functions are associated with poor control of malignancies or infections due to a lower expression of cytolytic molecules, such as granzymes and perforin. The altered phenotypes are characterized by the downregulation of certain activation receptors. NKG2D, CD16a, NKp30, NKp44, and NKp46 along with CD226 belong to the receptors that are usually decreased under settings of tumors or chronic infections. Another phenotypic feature is the upregulation of inhibitory receptors (Mamessier et al., 2011).

Therefore, many NK cell-based therapeutic approaches focus on the expansion and activation of fully functional NK cells and adoptive transfer to patients as patients would benefit from a better effector cell to tumor cell proportion. Previous studies were able to show that an in vivo expansion with cytokines represented a promising approach. An increment of circulating NK cells as well as an increased lytic function could be observed. Nevertheless, the in vivo expansion with cytokines led to high toxic side effects (Burns et al., 2003). In addition, the in vivo expansion with cytokines (especially IL-2) often showed an expansion of regulatory T cells (Treg cells) as well. These Treg cells can inhibit the NK cell activity (Gasteiger et al., 2013). To avoid these problems, new approaches used ex vivo expansion of NK cells. An advantage over the in vivo expansion is that the patients would not suffer from side effects. Furthermore, like mentioned before, under tumor conditions NK cells show an exhausted status that is characterized by an NK cell dysfunction. When NK cells were expanded in vivo in an immunosuppressive tumor environment, the NK cells tend to quickly show some kind of dysfunctions (Fujisaki et al., 2009). An ex vivo expansion offers the possibility of getting the NK cells out of the immunosuppressive tumor environment and to expand fully functional NK cells. Proof could be furnished that the infusion of allogenic NK cells induced clinical remission in patients with high-risk acute myeloid leukemia (Miller et al., 2005). Moreover, it could be observed that patients with other cancer types show a benefit from NK cell infusions (Terme, Ullrich, Delahaye, Chaput, & Zitvogel, 2008).

There are various ex vivo approaches available that are successful but represent a very costly approach when it comes to techniques or logistics. The ex vivo expansion with cytokines represents one appendage. The in vivo as well as ex vivo development and survival of NK cells require cytokines like IL-2, IL-4, IL-9, IL-15, IL-18 and IL-21 (Fujisaki et al., 2009; Miller et al., 2005). Moreover, the heterodimeric cytokine called natural killer cell stimulatory factor (NKSF) was able to boost NK cell-mediated cytotoxicity. It could be observed that autologous NK cells could be expanded and activated ex vivo. But in contrast it could be shown that the ex vivo expanded NK cells showed no clinical responses in cancer patients. Another approach is the expansion with a genetically modified K562 leukemia cell line. The modification involves the expression of a membrane-bound interleukin (IL)-15 and a 4-1BB ligand. The 4-1BB ligand specifically activates NK cells via the binding to CD137 on NK cells. The use of K562 cells showed a high and specific proliferation of human NK cells that demonstrated a high cytotoxicity against tumor cells. But the final cell product may contain traces of genetically modified organisms. By the same token, reinfused NK cell populations always contained tumor cells (Fujisaki et al., 2009). Various genetically modified feeder cells have been developed but in principle display the same limitations as described above. As an alternative bead-based approaches have been described. Similar considerations hold true for the ex vivo expansion of T cells and/or NKT cells.

Hence, there is still a need for means and methods that allow cost-efficient and simple expansion of NK cells, T cells and/or NKT cells, in particular such means and methods that obviate the need of genetically modified feeder cells or microbeads. This need is addressed by the present application.

Hence, the present invention relates in a first aspect to a fusion protein comprising (a) an antibody or an antibody fragment binding to an antigen being expressed on the surface of a target cell of NK cells, T cells and/or NKT cells, preferably the surface of a B cell or a tumor cell, (b) an antibody or an antibody fragment binding to 4-1BB, NKG2D, NKp30, NKp46, NKp44, CD28 DNAM1 or 2B4, and (c) IL-15, IL-2, IL-21, IL-18 or IL-12.

The fusion protein of the invention comprises the three components according to (a), (b) and (c).

Components (a) and (b) are each independently an antibody or an antibody fragment. These two antibodie(s) and/or antibody fragment(s) bind to different antigens. The antibody or an antibody fragment of (a) binds to an antigen being expressed on the surface of a target cell of NK cells or T cells/NKT cells (preferably the surface of a B cell or a tumor cell) while the antibody or an antibody fragment of (b) binds to 4-1BB, NKG2D, NKp30, NKp46, NKp44, CD28, 2B4 or DNAM-1.

The term “antibody” as used in accordance with the present invention comprises, for example, polyclonal or monoclonal antibodies. Furthermore, also fragments thereof, which still retain the required binding specificity to the target described herein. Antibody fragments comprise, inter alia, Fab or Fab′ fragments, Fd, F(ab′)2, Fv or scFv fragments, single domain VH or V-like domains, such as VhH or V-NAR-domains, as well as multimeric formats such as minibodies, diabodies, tribodies or triplebodies, tetrabodies or chemically conjugated Fab′-multimers (see, for example, Harlow and Lane “Antibodies, A Laboratory Manual”, Cold Spring Harbor Laboratory Press, 198; Harlow and Lane “Using Antibodies: A Laboratory Manual” Cold Spring Harbor Laboratory Press, 1999; Altshuler E P, Serebryanaya D V, Katrukha A G. 2010, Biochemistry (Mosc)., vol. 75(13), 1584; Holliger P, Hudson P J. 2005, Nat Biotechnol., vol. 23(9), 1126). The multimeric formats in particular comprise bispecific antibodies that can simultaneously bind to two different types of antigens. The first antigen can be found on the surface of a target cell of NK cells, T cells or NKT cells, preferably the surface of a B cell or a tumor cell. The second antigen can be found within one of the proteins 4-1BB, NKG2D, NKp30, NKp46, NKp44, CD28 or DNAM1 or 2B4. Non-limiting examples of bispecific antibodies formats are Biclonics (bispecific, full length human IgG antibodies), DART (Dual-affinity Re-targeting Antibody) and BiTE (consisting of two single-chain variable fragments (scFvs) of different antibodies) molecules (Kontermann and Brinkmann (2015), Drug Discovery Today, 20(7):838-847).

The term “antibody” also includes embodiments such as chimeric (human constant domain, non-human variable domain), single chain, humanised (human antibody with the exception of non-human CDRs) antibodies and human antibodies.

Various techniques for the production of antibodies are well known in the art and described, e.g. in Harlow and Lane (1988) and (1999) and Altshuler et al., 2010, loc. cit. Thus, polyclonal antibodies can be obtained from the blood of an animal following immunisation with an antigen in mixture with additives and adjuvants and monoclonal antibodies can be produced by any technique which provides antibodies produced by continuous cell line cultures. Examples for such techniques are described, e.g. in Harlow E and Lane D, Cold Spring Harbor Laboratory Press, 1988; Harlow E and Lane D, Using Antibodies: A Laboratory Manual, Cold Spring Harbor Laboratory Press, 1999 and include the hybridoma technique originally described by Kohler and Milstein, 1975, the trioma technique, the human B-cell hybridoma technique (see e.g. Kozbor D, 1983, Immunology Today, vol. 4, 7; Li J, et al. 2006, PNAS, vol. 103(10), 3557) and the EBV-hybridoma technique to produce human monoclonal antibodies (Cole et al., 1985, Alan R. Liss, Inc, 77-96). Furthermore, recombinant antibodies may be obtained from monoclonal antibodies or can be prepared de novo using various display methods such as phage, ribosomal, mRNA, or cell display. A suitable system for the expression of the recombinant (humanised) antibodies may be selected from, for example, bacteria, yeast, insects, mammalian cell lines or transgenic animals or plants (see, e.g., U.S. Pat. No. 6,080,560; Holliger P, Hudson P J. 2005, Nat Biotechnol., vol. 23(9), 11265).

Further, techniques described for the production of single chain antibodies (see, inter alia, U.S. Pat. No. 4,946,778) can be adapted to produce single chain antibodies specific for an epitope as defined in the above item (a) or (b). Surface plasmon resonance as employed in the BIAcore system can be used to increase the efficiency of phage antibodies.

Since components (a) and (b) of the fusion protein are each an antibody or an antibody fragment binding to a distinct antigen, the fusion protein of the invention may also be said to comprise a multispecific antibody (noting that the fusion protein may comprise yet further antibodies or antibody fragments) and in particular a bispecific antibody.

The term “multispecific antibody” as used in accordance with the present invention comprises binding motifs (e.g. antbodie(s) and/or antibody fragments) displaying binding specificity to the targets as defined in the above items (a) and (b). The multispecific antibody may also be extended by a third specificity binding a target on a further tumor or effector cell. The binding motifs of the at least two different monoclonal antibodies may be comprised in the multispecific antibody in the format of full-length antibodies but also as fragments thereof, which still retain the binding specificity to the target, for example an antigen being expressed on the surface of a tumor cell, are comprised in the term “antibody”.

In accordance with the present invention the multispecific antibody may have a multi-chain or single-chain format. Multi-chain or single-chain antibody formats are, for example, minibodies, diabodies, bibodies, tribodies or triplebodies, tetrabodies or chemically conjugated Fab′-multimers (see, for example, Harlow and Lane “Antibodies, A Laboratory Manual”, Cold Spring Harbor Laboratory Press, 1988; Harlow and Lane “Using Antibodies: A Laboratory Manual” Cold Spring Harbor Laboratory Press, 1999; Altshuler E P, Serebryanaya D V, Katrukha A G. 2010, Biochemistry (Mosc)., vol. 75(13), 1584; Holliger P, Hudson P J. 2005, Nat Biotechnol., vol. 23(9), 1126). Among the formats the bibody format is preferred since bibodies are illustrated by the examples. The bibody, a Fab-scFv fusion protein, is created by adding a scFv fragment to the C-terminus of Fab scaffold. In this class of multispecific antibodies the bispecific fragment utilizes the natural in vivo heterodimerization of the Fd fragment (the HC regions of Fab fragment) and light chain. The heterodimerization scaffold can be further incorporated with additional functions, such as scFvs, scaffold proteins, cytokines, etc. to form bivalent, bispecific molecules or trivalent, bi- or tri-specific molecules. The bibody molecules, Fab-L-scFv and Fab-H-scFv, are bispecific and bivalent. It has been shown that this format can retain the bispecific binding, a low tendency to aggregate and stable in physiological conditions. The multi-chain formats in particular comprise bispecific antibodies that can simultaneously bind to two different types of antigens. Non-limiting examples of bispecific antibody formats are Biclonics (bispecific, full length human IgG antibodies), DART (Dual-affinity Re-targeting Antibody) and BiTE (consisting of two single-chain variable fragments (scFvs) of different antibodies) molecules (Kontermann and Brinkmann (2015), Drug Discovery Today, 20(7):838-847). Further bispecific antibodies formats will be discussed herein below.

An antigen as used herein refers to a molecule or molecular structure being present on the outside of a cell, that can be specifically bound by an antibody or antibody fragment as comprises in the fusion protein of the invention. The antigen comprises an epitope (also called antigenic determinant), which is the part of an antigen that is recognized by the fusion protein of the invention.

As discussed, the antigens bound by the fusion protein of the invention can be found on the surface of a target cell of NK cells, T cells and/or NKT cells (preferably the surface of a B cell or a tumor cell) as well as within one of the proteins 4-1BB, NKG2D, NKp30, NKp46, NKp44, 2B4, CD28 and DNAM-1.

As also discussed above, NK (natural killer) cells are a type of cytotoxic lymphocyte. They are critical to the innate immune system that belong to the rapidly expanding family of known innate lymphoid cells (ILC) and represent 5-20% of all circulating lymphocytes in humans. The role of NK cells is analogous to that of cytotoxic T cells in the vertebrate adaptive immune response. NK cells provide rapid responses to virus-infected cell and other intracellular pathogens acting at around 3 days after infection and respond to tumor formation. Typically, immune cells detect the major histocompatibility complex (MHC) presented on infected cell surfaces, triggering cytokine release, causing the death of the infected cell by lysis or apoptosis. NK cells are unique, however, as they have the ability to recognize and kill stressed cells in the absence of antibodies and MHC, allowing for a much faster immune reaction. They were named “natural killers” because of the notion that they do not require prior activation to kill cells that are missing “self” markers of MHC class I. This role is especially important because harmful cells that are missing MHC I markers cannot be detected and destroyed by other immune cells, such as T lymphocyte cells. NK cells can be identified by the presence of CD56 and the absence of CD3 (CD56⁺, CD3⁻).

T cells, also called T lymphocytes, are a type of leukocytes (white blood cells) that are an essential part of the immune system. T cells are one of two primary types of lymphocytes—B cells being the second type—that determine the specificity of immune response to antigens (foreign substances) in the body. T cells originate in the bone marrow and mature in the thymus. In the thymus, T cells multiply and differentiate into helper, regulatory, or cytotoxic T cells or become memory T cells. They are then sent to peripheral tissues or circulate in the blood or lymphatic system. Once stimulated by the appropriate antigen, helper T cells secrete chemical messengers called cytokines, which stimulate the differentiation of B cells into plasma cells (antibody-producing cells). Regulatory T cells act to control immune reactions, hence their name. Cytotoxic T cells, which are activated by various cytokines, bind to and kill infected cells and cancer cells.

Natural killer T (NKT) cells are a heterogeneous group of T cells that share properties of both T cells and natural killer cells. Many of these cells recognize the non-polymorphic CD1d molecule. NKT cells constitute only approx. 1% of all peripheral blood T cells that also express a variety of markers that are typical for NK cells, such as CD16 and CD56.

The target cells of NK cells, T cells and/or NTK cells are not particularly limited. NK cells can, for example, target and kill aberrant cells, such as virally infected and tumorigenic cells. Killing is mediated by cytotoxic molecules which are stored within secretory lysosomes, a specialized exocytic organelle found in NK cells. The target cells can also be antigen presenting cell (APCs), including dendritic cells (DC). Similarly, target cells of T cells include cells infected with intracellularly replicating pathogens, tumor cells and foreign cells entering the body as part of a tissue transplant.

The target cell is preferably a B cell or a tumor cell.

B cells (B lymphocytes) are a type of white blood cell of the lymphocyte subtype. They function in the humoral immunity component of the adaptive immune system. B cells produce antibody molecules which may be either secreted or inserted into the plasma membrane where they serve as a part of B-cell receptors. When a naïve or memory B cell is activated by an antigen, it proliferates and differentiates into an antibody-secreting effector cell, known as a plasmablast or plasma cell. Additionally, B cells present antigens (they are also classified as professional antigen-presenting cells (APCs)) and secrete cytokines. B cells, unlike the other two classes of lymphocytes, T cells and natural killer cells, express B cell receptors (BCRs) on their cell membrane. BCRs allow the B cell to bind to a foreign antigen, against which it will initiate an antibody response.

Tumor cells are aberrant cells that differ from normal body cells in many ways. Normal cells become tumor cells when a series of mutations leads the cell to continue to grow and divide out of control. Also, unlike normal cells, tumor cells may have the ability to invade nearby tissues and/or spread to distant regions of the body. The series of mutations often results in the expression of an antigen on the surface of a tumor cell that is not expressed on normal cells. In addition, tumor cells express various antigens found also on heathy tissues. However, also such antigens can be used as target structures, given that they have a limited expression pattern and/or their expression is restricted to certain tissues or cell types in heathy tissues. Such antigens are referred to herein as antigens being expressed on the surface of a tumor cell. The antigen is preferably not expressed on normal cells. The “bispecific binding antibody” may thus specifically or at least highly preferentially bind to tumor cells. The tumor as referred to herein may be malignant or benign tumor. The tumor is preferably a malignant tumor, which is also referred to herein as cancer.

As will be explained in more detail in the following all of the proteins 4-1BB, NKG2D, NKp30, NKp46, NKp44, 2B4, CD28 or DNAM-1 are all expressed on the surface of NK cells, T cells and/or NKT cells and they are all involved in the activation of these cells.

4-1BB (ILA/CD137) is a member of the tumor necrosis factor receptor family, expressed on activated T lymphocytes and NK cells.

NKG2D is a transmembrane protein belonging to the NKG2 family of C-type lectin-like receptors. NKG2D plays a key role in immune surveillance of tumors and pathogens. In humans, NKG2D is expressed by NK cells and cytotoxic thymocytes and recognizes “induced-self proteins”, which are frequently expressed at the cell surface after viral infection or malignant transformation. Human NKG2D ligands include MHC class I-related chain (MIC) A and B as well as UL16-binding proteins (ULBP) 1-6. Recognition of these danger signaling antigens results in cell activation through an intracellular activation pathway via the NKG2D-associated adapter protein DNAX-activating protein of 10 kDa (DAP10). In NK cells, this signal promotes natural cytotoxicity.

NKp30 (CD337) is a stimulatory receptor on human NK cells implicated in tumor immunity and is capable of promoting or terminating dendritic cell maturation.

NKp46 is a major NK cell-activating receptor that is involved in the elimination of target cells being killed by NK cells.

NKp44 (CD336) is a member of Natural Cytotoxicity Receptors (NCRs). It is an activating receptor playing a crucial role in most functions exerted by activated NK cells and also by other NKp44+ immune cells.

DNAM-1 (CD226) is an activating receptor belonging to the Ig superfamily and is constitutively expressed by most NK cells, T cells, macrophages, and DCs. DNAM-1 interacts with LFA-1, required for its functional activity on both NK and cytotoxic T cells. Ligands for DNAM-1 (DNAM1Ls) include Nectin-2/CD112 and PVR/CD155 belonging to the Nectin/Nectin-like family of adhesion molecules. The activating effects of DNAM-1 can be counteracted by TIGIT, a recently identified inhibitory receptor binding to PVR, and expressed by T and NK cells.

2B4 (CD244) is a protein expressed on NK cells and some T cells. The interaction between NK-cell and target cells via this receptor is thought to modulate NK-cell cytolytic activity.

CD28 is a protein expressed on T cells that provides co-stimulatory signals required for T cell activation and survival.

It is of note that an antibody or an antibody fragment binding to CD16 is not suitable as second component (b) of the fusion protein of the invention, because the fusion protein shall be suitable to expand NK cells, in particular NK cells with high ADCC activity. CD16a (FcγRIIIa) is regarded as the strongest cytotoxic trigger on NK cells. Although high affinity binding CD16a in the context of triggering strong cytotoxicity against target cells may be desirable, in the context of the fusion protein of the invention a high affinity CD16a binding domain is not desired. This is because it is well established that cross-linking CD16a with antibodies downregulates CD16a expression. This downregulation is expected to result in NK cell preparations being compromised in terms of ADCC induction. The dramatic effect of CD16a binding by a CD16 antibody in contrast to natural IgG is described, for example, in Romee et al. (2013), Blood, (18):3599-608 and Capuano et al. (2017), Oncoimmunology, 6(3):e1290037).

As the third component (c) the fusion protein of the invention comprises an interleukin selected from the group costing of IL-15, IL-2, IL-18, IL-21 or IL-12. Among this list IL-15 is most preferred.

Also contemplated herein are as the third component (c) of the fusion protein of the invention mutants, truncated versions or variants of an interleukin selected from the group costing of IL-15, IL-2, IL-18, IL-21 or IL-12. The primary functions of interleukins are to modulate cell growth, cell differentiation, and activation during inflammatory and immune responses and one or more of these functions are preferably retained in the mutants, truncated versions or variants of an interleukin selected from the group costing of IL-15, IL-2, IL-18, IL-21 or IL-12.

Interleukins (ILs) are a group of cytokines (secreted proteins and signal molecules) that are expressed and secreted by white blood cells (leukocytes) as well as some other body cells. The human genome encodes more than 50 interleukins and related proteins.

IL-15, IL-2, IL-21, IL-18 or IL-12 are all so-called proinflammatory cytokines. Proinflammatory cytokines are produced predominantly by activated macrophages and are involved in the upregulation of inflammatory reactions. NK cells respond to certain cytokines and their activity can be enhanced by stimulation with IL-15, IL-2, IL-18, IL-21 or IL-12 (de Rham et al. (2007), Arthritis Res Ther; 9(6): R125 and Rezani and Rouce (2015), Front. Immunol.).

Interleukin-15 (IL-15) is a cytokine with structural similarity to Interleukin-2 (IL-2). Like IL-2, IL-15 binds to and signals through a complex composed of IL-2/IL-15 receptor beta chain (CD122) and the common gamma chain (gamma-C, CD132). IL-15 is secreted by mononuclear phagocytes (and some other cells) following infection by virus(es). This cytokine induces the proliferation of natural killer cells, i.e. cells of the innate immune system whose principal role is to kill virally infected cells. IL-15R is expressed by NK cells.

Interleukin-2 (IL-2) is an interleukin, a type of cytokine signalling molecule in the immune system. It is a 15.5-16 kDa protein that regulates the activities of white blood cells (leukocytes, often lymphocytes) that are responsible for immunity. IL-2 is part of the body's natural response to microbial infection, and in discriminating between foreign (“non-self”) and “self”. IL-2 mediates its effects by binding to IL-2 receptors, which are expressed by lymphocytes. The major sources of IL-2 are activated CD4+ T cells and activated CD8+ T cells. Dimeric IL-2R is expressed by memory CD8+ T cells and NK cells, whereas regulatory T cells and activated T cells express high levels of trimeric IL-2R.

Interleukin-18 (IL-18) is a cytokine also being known as interferon-gamma inducing factor. IL-18 is a proinflammatory cytokine. Many cell types, both hematopoietic cells and non-hematopoietic cells, have the potential to produce IL-18. IL-18 production was first recognized in Kupffer cells, liver-resident macrophages. However, IL-18 is constitutively expressed in non-hematopoietic cells, such as intestinal epithelial cells, keratinocytes, and endothelial cells. IL-18 can modulate both innate and adaptive immunity and its dysregulation can cause autoimmune or inflammatory diseases.

Interleukin-21 (IL-21) is a cytokine that has potent regulatory effects on cells of the immune system, including natural killer (NK) cells and cytotoxic T cells that can destroy virally infected or cancerous cells. This cytokine induces cell division/proliferation in its target cells. The IL-21 receptor (IL-21R) is expressed on the surface of T, B and NK cells.

Interleukin 12 (IL-12) is an interleukin that is naturally produced by dendritic cells, macrophages, neutrophils, and human B-lymphoblastoid cells (NC-37) in response to antigenic stimulation. IL-12 plays an important role in the activities of natural killer cells and T lymphocytes. IL-12 mediates enhancement of the cytotoxic activity of NK cells and CD8+ cytotoxic T lymphocytes. There also seems to be a link between IL-2 and the signal transduction of IL-12 in NK cells. IL-2 stimulates the expression of two IL-12 receptors, IL-12R-β1 and IL-12R-β2, maintaining the expression of a critical protein involved in IL-12 signalling in NK cells. Enhanced functional response is demonstrated by IFN-γ production and killing of target cell. The IL-12R is expressed by NK cells.

It is of note the order of components (a), (b) and (c) as well as the way of connecting the components are not particularly limited as long as the two antibodies or antibody fragments of components (a) and (b) can bind their antigens and component (c) can bind the interleukin receptor on the NK cells and their target cells. The order of components can be, for example, from the N-terminus to the C-terminus (a) (b) (c) but also can be (c) (b) (a), (c) (a) (b), (b) (a) (c), (b) (c) (a) or (a) (c) (b).

The components are preferably connected via a linker, wherein the linker is preferably a flexible peptide linker as will be further described herein below.

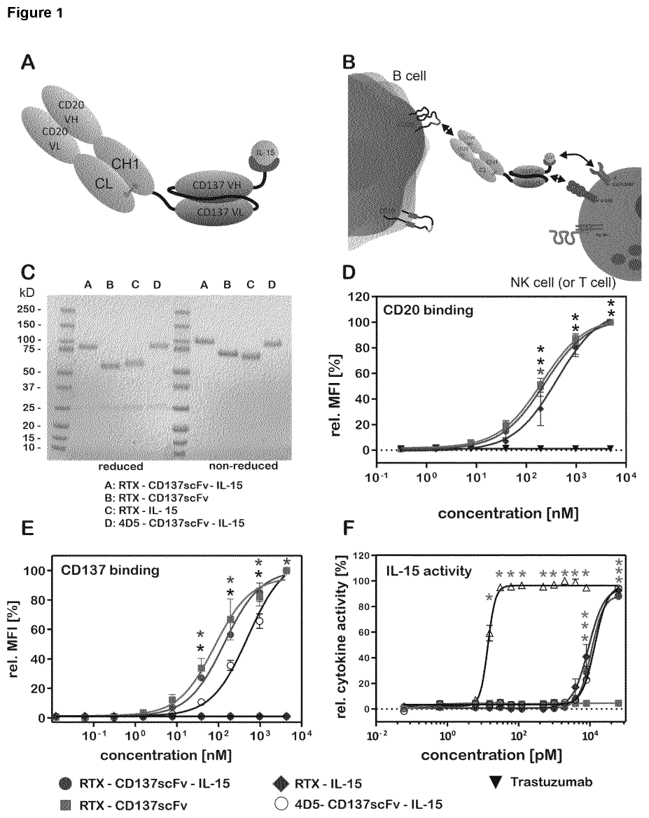

As can be taken from the above explanations, the fusion protein of the invention via its antibody components (a) binds to target cells and provides transpresentation of component c) of the fusion protein, IL-15, IL-2, IL-18, IL-21 or IL-12 to bind to the receptor of the respective interleukin on the surface of the NK-cells, T cells and/or NTK cells. This likely in turn leads to upregulation of the target antigen of component (b) which further connects the NK cells, T cells and/or NTK cells and the target cells of NK cells, T cells and/or NTK cells; see FIG. 1B. The co-binding of 4-1BB, NKG2D, NKp30, NKp46, NKp44, 2B4, CD28 or DNAM-1 by component (b) and the IL-15R, IL-2R, IL-18R, IL-21R or IL-12R by component (c) of the fusion protein on the surface of NK cells triggers two signal cascades that activate NK cells, T cells and/or NTK cells thereby leading to their proliferation. The connection of the NK cells, T cells and/or NTK cells and their target cells further improves the NK cell, T cell and/or NTK cell proliferation presumably in view of positive feed-back loops between the target cells and the NK cells, T cells and/or NTK cells by providing hyper crosslinking of component b) and c) and thereby stronger transpresentation of the cytokine. In addition, the close proximity of the NK cells, T cells and/or NTK cells and their target cells improves the killing efficacy of malignant target cells by the NK cells, T cells and/or NTK cells but to a lesser extent of non-malignant cells.

As is shown in the appended examples herein below based on the exemplified fusion protein of the invention consisting of one or two (DuoFab) CD20-directed Fab-fragment(s) as component (a), an agonistic anti-4-1BB (CD137) scFv as component (b), and the sushi domain of the IL-15 receptor fused to human IL-15 as component (c) (RTX-CD137scFv-IL-15/RTX-DuoFab-CD137scFv-IL-15) triggers strong NK cell expansion when bound to an NK target cell (which are exemplified autologous B cells). This is believed to be due to the binding of the fusion protein to IL-15R and 4-1BB on the surface of the NK cell. The essentially same is shown for the further exemplified fusion proteins of the invention consisting of (a) one or two (DuoFab) BCMA-directed Fab-fragment(s), an agonistic anti-4-1BB (CD137) scFv, and the sushi domain of the IL-15 receptor fused to human IL-15 (BCMA-CD137scFv-IL-15/BCMA-DuoFab-CD137scFv-IL-15), (b) CD19-CD137scFv-IL-15, (c) RTX-CD137scFvdss-IL-15, (d) RTX-CD137scFv-IL-2, (e) RTX-NKp46scFv-IL-15, and (f) (RTX-NKG2DscFv-IL-15. The data on CD20, BCMA and CD19 as component (a), CD137 (optionally with addition of a disulfide bond), NKp46 and NKG2D as component (b) and IL-15 (with sushi domain) and IL-2 as component (c) show that the all three components can be varied. Strong NK cell expansion was obtained with all these exemplified fusion proteins. The NK cell expansion and the cytolytic activity as obtained by the exemplified fusion proteins was in particular significantly stronger as compared to the prior art molecule design (SEQ ID NOs 79 and 80) as described in Kerner, Mol Cancer Ther, 2014; Beha, Mol Cancer Ther, 2019. This prior art molecule design is based on a 4-1BB ligand in order to stimulate to stimulate CD137 instead of using an CD137 scFv. The data with one or two Fab-fragments shows that the particular formats of the antibodies as used in the fusion protein of the invention is also not limited. No significant differences in terms of NK cell expansion and cytotoxic activity of the expanded NK cells were observed between one and two Fab fragments.

The examples herein below show that all three components (a), (b) and (c) of the fusion protein of the invention were necessary for full activity. Expansion rates ranged between 10-10,000-fold. The expanded NK cells showed high cytotoxic capacity against a wide range of tumor cell lines representing various tumor entities. Importantly, the expanded highly activated NK cells did not attack non-malignant B cells indicating that NK cells expanded by the fusion protein of the invention are still physiologically regulated. The cytotoxic activity of the expanded NK cells can be further enhanced by combination with a therapeutic antibody, e.g. in the format of a bispecific antibody. The fusion protein of the invention is also able to expand NK cells from Multiple Myeloma patients to a high extent.

In accordance with a preferred embodiment of the first aspect of the invention the antigen of (a) is selected from the group consisting of CD20, BCMA, CD19, CD22, CD37, CD38, CD7, CD33, CD44, CD54, CD64, CD75s, CD79b, CD96, CD123, CD317, CD319, FCRL5, EGFR, B7-H3, HER2, EpCAM, CEA, GD2 and Claudin 6/18, Trop-2, ROR1, PSMA, FoIR1, STEAP1, Her3, uPAR, Muc-1, cMet, CXCR4, SAP-1, Muc-16, TAG-72, HLA-DR, CD30, DLL4, CD221, Mesothelin, GPRC5D, Nectin-4, LIV-1, and Tissue factor, and is preferably CD20 or BCMA.

The fusion protein of the invention may include binding domains to more than one of these antigens. The fusion protein of the invention may accordingly bind to two or more antigens selected from the group consisting of CD20, BCMA, CD19, CD22, CD37, CD38, CD7, CD33, CD44, CD54, CD64, CD75s, CD79b, CD96, CD123, CD138, CD317, CD319, FCRL5, EGFR, B7-H3, HER2, EpCAM CEA, Claudin 6/18, ROR1, PSMA, FolR1, STEAP1, Her3, uPAR, Muc-1, cMet, CXCR4, SAP-1, Muc-16, TAG-72, HLA-DR, CD30, DLL4, CD221, Mesothelin, GPRC5D, Nectin-4, LIV-1 and Tissue factor wherein the two or more antigens preferably comprise CD20 and/or BCMA. The Expression of all these antigens is known to be implicated in cancer development and all these antigens can be found on certain cancer cells.

B-lymphocyte antigen CD20 or CD20 is expressed on the surface of all B-cells beginning at the pro-B phase (CD45R+, CD117+) and progressively increasing in concentration until maturity. CD20 has been found on B-cell lymphomas, hairy cell leukemia, chronic lymphocytic leukemia (CLL), B-cell acute lymphoblastic leukemia (ALL) and melanoma cancer stem cells.

The B-cell maturation antigen (BCMA), also known as tumor necrosis factor receptor superfamily member 17 (TNFRSF17), is a protein that in humans is encoded by the TNFRSF17 gene. TNFRSF17 is a cell surface receptor of the TNF receptor superfamily which recognizes B-cell activating factor (BAFF). BCMA recognizes B-cell activating factor (BAFF). BCMA expression is implicated in leukemia, lymphomas, and multiple myeloma.

CD19 is a transmembrane protein being expressed in B cells. Since CD19 is a marker of B cells, the protein has been used to diagnose and target cancers that arise from this type of cell, notably B cell lymphomas, acute lymphoblastic leukemia (ALL), and chronic lymphocytic leukemia (CLL).

CD22 is a molecule belonging to the SIGLEC family of lectins and is found on the surface of mature B cells and to a lesser extent on some immature B cells. Also CD22 has been used to diagnose and target cancers that arise from B cells, such as acute lymphoblastic leukemia (ALL).

CD37 is a member of the transmembrane 4 superfamily. The expression of CD37 is restricted to cells of the immune system, with highest abundance on mature B cells, and lower expression is found on T cells and myeloid cells. In cancer, CD37 is highly expressed on malignant B cells in a variety of B-cell lymphomas and leukemias, including Non-Hodgkin lymphoma (NHL) and CLL.

CD38 is a glycoprotein found on the surface of many immune cells (white blood cells), including CD4+, CD8+, B lymphocytes and natural killer cells. CD38 is also expressed in various hematological malignancies including NHL, MM, CLL and ALL.

CD7 encodes a transmembrane protein which is a member of the immunoglobulin superfamily. CD7 is found on thymocytes and mature T cells. CD7 is expressed by T lineage leukemias and lymphomas and is a leukemic prognostic marker.

CD33 is a transmembrane receptor being expressed on cells of myeloid lineage. It is a target used for treatment of patients with acute myeloid leukemia.

CD44 is a cell-surface glycoprotein being involved in cell-cell interactions, cell adhesion and migration. CD44 is expressed in a large number of mammalian cell types. Variations in CD44 are reported as cell surface markers for some breast and prostate cancer stem cells.

CD54 is a cell surface glycoprotein which is typically expressed on endothelial cells and cells of the immune system. CD54 has an important role in ocular allergies recruiting pro-inflammatory lymphocytes and mast cells promoting a type I hypersensitivity reaction.

CD64 is a type of integral membrane glycoprotein known as an Fc receptor that binds monomeric IgG-type antibodies with high affinity. CD64 is found on macrophages and monocytes. Neutrophil CD64 expression is increased in inflammatory autoimmune diseases.

CD75s is an alpha-2,6-sialylated carbohydrate epitope being expressed by mature B cells (especially germinal centre B cells), red blood cells and some epithelial cells. CD75s has been identified as a promising target for immunotherapy of mature B cell malignancies.

CD79b is the B-cell antigen receptor complex-associated protein beta chain. Diseases associated with CD79b include agammaglobulinemia 6, autosomal recessive and agammaglobulinemia, Non-Bruton type.

CD96 is a transmembrane glycoprotein that has three extracellular immunoglobulin-like domains and is expressed by resting NK cells. CD96 has been reported to correlate with immune profiles and the clinical outcome of gliomas.

CD123 is a molecule found on cells which helps transmit the signal of interleukin-3, a soluble cytokine important in the immune system, such as pluripotent progenitor cells of hematopoietic cells. CD123 is expressed across acute myeloid leukemia (AML) subtypes, including leukemic stem cells.

CD138 (or syndecan 1) is a protein which in humans is encoded by the SDC1 gene. The protein is a transmembrane (type I) heparan sulfate proteoglycan. CD138 functions as an integral membrane protein and participates in cell proliferation, cell migration and cell-matrix interactions via its receptor for extracellular matrix proteins. CD138 is a sponge for growth factors and chemokines, with binding largely via heparan sulfate chains.

CD317 is a lipid raft associated protein being expressed in mature B cells, plasma cells and plasmacytoid dendritic cells, and in many other cells. It is only expressed as a response to stimuli from the IFN pathway. Several reports have described the expression of CD317 in various types of malignancies, including lung cancer, leukemia, and lymphoma.

CD319 (also known as CS1 (CD2 subset-1), CRACC and SLAMF7) is a single-pass type I transmembrane glycoprotein, expressed on NK cells, subsets of mature dendritic cells, activated B cells, and cytotoxic lymphocytes, but not in promyelocytic, B or T cell lines. CD319 is a robust marker of normal plasma cells and malignant plasma cells in multiple myeloma.

FCRL5 (Fc receptor-like protein 5, also known as CD307) is a receptor that recognizes intact IgG, possibly enabling B cells to sense Ig quality. Diseases associated with FCRL5 include hairy cell leukemia and lymphoma.

EGFR (epidermal growth factor receptor) is a transmembrane protein that is a receptor for members of the epidermal growth factor family (EGF family) of extracellular protein ligands. In many cancer types, mutations affecting EGFR expression or activity could result in cancer.

B7-H3 (or CD 276) is a 316 amino acid-long type I transmembrane protein, existing in two isoforms determined by its extracellular domain. In mice, the extracellular domain consists of a single pair of immunoglobulin variable (IgV)-like and immunoglobulin constant (IgC)-like domains, whereas in humans it consists of one pair (2Ig-B7-H3) or two identical pairs (4Ig-B7-H3) due to exon duplication. In non-malignant tissues, B7-H3 has a predominantly inhibitory role in adaptive immunity, suppressing T cell activation and proliferation.

HER2 (Receptor tyrosine-protein kinase erbB-2, also known as CD340) is a receptor having an important role in normal cell growth and differentiation. HER2 over-expression is known to occur, for example, in breast, ovarian, stomach, adenocarcinoma of the lung, and uterine cancer.

EpCAM (epithelial cell adhesion molecule) is a transmembrane glycoprotein mediating Ca2+-independent homotypic cell-cell adhesion in epithelia. EpCAM is overexpressed in many carcinomas and in cancer stem cells, making EpCAM an attractive target for immunotherapy.

CEA (Carcinoembryonic antigen) describes a set of highly related glycoproteins involved in cell adhesion. CEA is normally produced in gastrointestinal tissue during fetal development, but the production stops before birth. In adults, CEA is primarily expressed in cells of (malignant and benign) tumors.

GD2 is a disialogangliside with limited expression in healthy tissues. In certain tumors, GD2 is extensively expressed and has been associated with cancer development. The antigen is a target in the treatment of neuroblastoma.

CLDNs (claudins) refers to the members of a family of proteins which, along with occludin, are the most important components of the tight junctions (zonulae occludentes). Altered expression of several claudin proteins, in particular claudin-1, -3, -4 and -7, has been linked to the development of various cancers. In addition, CLDN6 and CLDN18.2 are attractive targets for immunotherapy.

Tumor-associated calcium signal transducer 2, also known as Trop-2 and as epithelial glycoprotein-1 antigen (EGP-1). Trop-2 is a carcinoma-associated antigen defined by the monoclonal antibody GA733. This antigen is a member of a family including at least two type I membrane proteins. It transduces an intracellular calcium signal and acts as a cell surface receptor.

Tyrosine-protein kinase transmembrane receptor ROR1, also known as neurotrophic tyrosine kinase, receptor-related 1 (NTRKR1), is an enzyme. ROR1 is a member of the receptor tyrosine kinase-like orphan receptor (ROR) family. The protein modulates neurite growth in the central nervous system.

PSMA (Prostate-specific membrane antigen) is a membrane protein that is highly expressed on prostate adenocarcinomas, with limited expression in benign and extraprostatic tissues. Therefore, it represents a promising target in prostate cancer.

Folate receptor 1 (FOLR1) is a transmembrane protein overexpressed in selected solid tumors, e.g. in over one-third of gastric cancer patients. It is rarely expressed in normal tissue.

STEAP1 (six-transmembrane epithelial antigen of prostate-1) is expressed in about 90% of prostate cancers, and also in other malignancies. STEAP1 is associated with tumor invasiveness and progression and only expressed at low levels in normal tissues.

Her3 is a heterodimeric partner for other EGFR family members and has the potential to regulate EGFR/HER2-mediated resistance. Upregulation of HER3 is associated with several malignancies and promotes tumor progression by interacting with other receptor tyrosine kinases.

uPAR (Urokinase-type plasminogen activator receptor, CD87) belongs to the lymphatic antigen-6 superfamily. The uPAR receptor is a single-chain membrane glycoprotein receptor and is anchored to the cell membrane by a glycosylphosphatidylinositol (GPI) linkage. It is expressed at low levels in healthy tissues and at high levels in malignant tumours. uPAR shows high expression in solid tumour tissues, such as breast, lung, ovarian and prostate as well as several other entities including several haematologic malignancies.

Muc-1 is specifically overexpressed and aberrantly glycosylated in many types of cancers, e.g. gastrointestinal cancers.

cMet is aberrantly expressed in various malignancies, particularly in non-small cell lung cancer, gastrointestinal cancer, and hepatocellular carcinoma.

CXCR4 (C—X—C motif chemokine receptor 4) upregulation in a variety of cancer entities is widely acknowledged, rendering this receptor as suitable target for solid tumors, including adrenocortical carcinoma or small-cell lung cancer.

SAP-1 (stomach-cancer-associated protein tyrosine phosphatase 1) is a human transmembrane-type protein tyrosine phosphatase. SPA-1 expression is abundant in colon and pancreatic cancer cells.

Aberrant overexpression of Muc-16 (CA125) has been observed in several malignancies, including ovarian, pancreatic, breast, and lung cancer. Due to the aberrant overexpression, Muc-16 has emerged as potential target in immunotherapy.

TAG-72 (Tumor-associated glycoprotein 72 antigen) is found at high levels on the surface of several cancer types, including ovarian cancer.

HLA-DR (the human leukocyte antigen-DR) is 1 of 3 polymorphic isotypes of the class II major histocompatibility complex antigen. Because HLA-DR is expressed at high levels on a range of hematologic malignancies, it is an interesting target for antibody-based lymphoma therapy.

CD30 is a member of the tumor necrosis factor receptor superfamily, that is expressed in certain hematopoietic malignancies, including cutaneous T cell lymphoma, anaplastic large cell lymphoma and Hodgkin lymphoma. It is an established target for antibody-based immunotherapy (e.g. Brentuximab-Vedotin).

DLL4 (delta like canonical Notch ligand 4). he delta gene family encodes Notch ligands that are characterized by a DSL domain, EGF repeats, and a transmembrane domain. DLL4 is associated with gastric cancer.

CD221 (IGF-1R) is an important tyrosine kinase receptor that plays an important role in mitogenesis, angiogenesis, transformation, apoptosis, and cell motility. A variety of preclinical and epidemiological studies have identified a role of IGF-1R in carcinogenesis, including cancers of the prostate, breast, colorectum and lung.

Mesothelin expression has been detected in many solid tumors, such as ovarian cancer, pancreatic adenocarcinoma, lung and uterine malignancies as well as cholangiocarcinoma. More recently mesothelin has also been discussed as a therapeutic target in AML.

GPRC5D (G protein-coupled receptor, class C, group 5, member D) is one of the members of the G protein-coupled receptor (GPCRs) family and a therapeutic target in multiple myeloma.

The Nectin cell adhesion protein 4 (Nectin-4) is overexpressed in different human malignancies and aberrant expression has been correlated with cancer progression. Nectin-4 is overexpressed in urothelial carcinoma and several other malignancies.

LIV-1 is a member of the solute carrier family 39; a multi-span transmembrane protein with metalloproteinase activity. It serves as a therapeutic target for the treatment of metastatic breast cancer.

Tissue factor is expressed by a variety of cancers as serves as a target for antibody-based therapeutic approaches.

In accordance with a preferred embodiment of the first aspect of the invention each of the antibody fragment of (a) and (b) is independently selected from Fab, scFv, Fv, VHH and dAb, and wherein the antibody fragment of (a) is preferably Fab and the antibody fragment of (b) is preferably scFv.

While the particular format of the fusion protein of the invention is not particularly limited, the fusion protein in accordance with this preferred embodiment comprises as the antibody fragment of (a) and (b) each independently a Fab, scFv, Fv, VHH, or dAb.

Fab (fragment antigen binding), scFv (single-chain fragment variable), Fv (fragment variable), VHH (variable domain of a heavy only antibody) and dAb (domain antibody) are well-known fragments of a full (or complete) antibody. A full (or complete) antibody consists of each two copies of the entire light and heavy immunoglobulin chains. Among this list of antibody fragments a scFv fragment is particularly preferred as being comprised in the fusion protein of the invention.

The distinguishing properties of antibody fragments as compared to full-length antibodies are, for example, a smaller size, monovalent antigen binding, lack of FcR binding, general lack of complex glycosylation and/or robust biophysical properties.

The format of the fusion protein of the invention preferably comprises an IgG and scFv or a Fab and scFv and more preferably a IgG-scFv or a Fab-scFv fusion protein as components (a) and (b) or vice versa. In the first case an IgG (i.e. full IgG antibody) is fused to a scFv fragment and in the second case a Fab fragment is fused to a scFv fragment.

For example, the fusion protein of the invention may comprise a scFv fragment, said scFv fragment is preferably fused via a flexible peptide-linker or a preferred example thereof as will be described herein below.

In accordance with the preferred option of the above embodiment of the first aspect of the invention the Fab scaffold specifically binds to the antigen of (a) and the scFv fragment specifically binds to the antigen of (b).

The Fab scaffold can either be single Fab fragment or two Fab-fragments (also called DuoFab herein). This particular Fab-scFv format as comprised in the fusion protein of the invention is illustrated in the examples of the application as filed (single Fab as well as DuoFab) and, thus, particularly preferred. The Fab-scFv format with an intermediate molecular mass of about 75 kDa may—in contrast to the tandem scFv format—not be eliminated by renal clearance thereby prolonging its in vivo half-life. Compared to IgG-like formats the smaller size displays favorable characteristics in mediating synapse formation between target and effector cell. Obviating the use of multiple scFv fragments such Fab-scFv molecules show less tendency to form multimers or aggregates.

In case even further prolonged in vivo half-life is desired the Fab-scFv format can be equipped in addition with an Fc domain. Such molecules with a molecular mass of about 125 kDa are still smaller than regular IgG antibodies and may therefore also demonstrate favorable characteristics in terms of tissue penetration.

In this connection it is to be understood that the Fc scaffold does not comprise an antigen binding site but is a further component of the fusion. The Fc scaffold can, for example, increase the in vivo serum stability and retention time of the multispecific antibody.

Also, in case of in vivo applications, if desired, the Fab-scFv format can be a DuoFab-scFv format. Such molecules display increased retention time (see Example 3) which is advantageous for in vivo applications.

As discussed herein above, the exemplified fusion proteins of the invention comprise as component (a) one or two (DuoFab) CD20-directed Fab-fragment(s) (also cladded RTX=rituximab herein), one or two (DuoFab) BCMA-directed Fab-fragment(s) (also called BCMA herein), or a CD19-directed Fab-fragment.

The light chain DNA and protein sequences of RTX are SEQ ID NOs 1 and 2 and the heavy chain VH DNA and protein sequences of RTX are SEQ ID NOs 3 and 4.

It is therefore preferred that component (a) comprises (a) sequence(s) being with increasing preference at least 80%, at least 85%, at least 90%, at least 95%, at least 98%, at least 99%, and 100% identical to SEQ ID NOs 2 and/or 4, or is encoded by (a) sequence(s) being with increasing preference at least 80%, at least 85%, at least 90%, at least 95%, at least 98%, at least 99%, and 100% identical to SEQ ID NOs 1 and/or 3.

The light chain DNA and protein sequences of BCMA are SEQ ID NOs 5 and 6 and the heavy chain VH DNA and protein sequences of BCMA are SEQ ID NOs 7 and 8.

It is therefore also preferred that component (a) comprises (a) sequence(s) being with increasing preference at least 80%, at least 85%, at least 90%, at least 95%, at least 98%, at least 99%, and 100% identical to SEQ ID NOs 6 and/or 8, or is encoded by (a) sequence(s) being with increasing preference at least 80%, at least 85%, at least 90%, at least 95%, at least 98%, at least 99%, and 100% identical to SEQ ID NOs 5 and/or 7.

The light chain DNA and protein sequences of the CD19-directed Fab-fragment are SEQ ID NOs 47 and 48 and the heavy chain VH DNA and protein sequences of the CD19-directed Fab-fragment are SEQ ID NOs 49 and 50.

It is therefore preferred that component (a) comprises (a) sequence(s) being with increasing preference at least 80%, at least 85%, at least 90%, at least 95%, at least 98%, at least 99%, and 100% identical to SEQ ID NOs 48 and/or 50, or is encoded by (a) sequence(s) being with increasing preference at least 80%, at least 85%, at least 90%, at least 95%, at least 98%, at least 99%, and 100% identical to SEQ ID NOs 47 and/or 49.

As discussed herein above, the exemplified fusion proteins of the invention comprise as component (b) an agonistic anti-4-1BB (CD137) scFv (also called CD137 herein), an agonistic NKp46 scFv, an agonistic NKG2D scFv or an agonistic anti-4-1BB (CD137) scFv dss (with an additional disulfide bond), whereby the agonistic anti-4-1BB (CD137) scFv is preferred.

The light chain VL DNA and protein sequences of CD137 scFv are SEQ ID NOs 9 and 10 and the heavy chain VH DNA and protein sequences of CD137 scFv are SEQ ID NOs 11 and 12.

It is therefore preferred that component (b) comprises (a) sequence(s) being with increasing preference at least 80%, at least 85%, at least 90%, at least 95%, at least 98%, at least 99%, and 100% identical to SEQ ID NOs 10 and/or 12, or is encoded by (a) sequence(s) being with increasing preference at least 80%, at least 85%, at least 90%, at least 95%, at least 98%, at least 99%, and 100% identical to SEQ ID NOs 9 and/or 11.

The light chain VL DNA and protein sequences of NKp46 scFv are SEQ ID NOs 51 and 52 and the heavy chain VH DNA and protein sequences of NKp46 scFv are SEQ ID NOs 53 and 54.

It is therefore preferred that component (b) comprises (a) sequence(s) being with increasing preference at least 80%, at least 85%, at least 90%, at least 95%, at least 98%, at least 99%, and 100% identical to SEQ ID NOs 52 and/or 54, or is encoded by (a) sequence(s) being with increasing preference at least 80%, at least 85%, at least 90%, at least 95%, at least 98%, at least 99%, and 100% identical to SEQ ID NOs 51 and/or 53.

The light chain VL DNA and protein sequences of NKG2D scFv are SEQ ID NOs 55 and 56 and the heavy chain VH DNA and protein sequences of NKG2D scFv are SEQ ID NOs 57 and 58.

It is therefore preferred that component (b) comprises (a) sequence(s) being with increasing preference at least 80%, at least 85%, at least 90%, at least 95%, at least 98%, at least 99%, and 100% identical to SEQ ID NOs 56 and/or 58, or is encoded by (a) sequence(s) being with increasing preference at least 80%, at least 85%, at least 90%, at least 95%, at least 98%, at least 99%, and 100% identical to SEQ ID NOs 55 and/or 57.

The light chain VL DNA and protein sequences of CD137 scFv dss are SEQ ID NOs 59 and 60 and the heavy chain VH DNA and protein sequences of CD137 scFv dss are SEQ ID NOs 61 and 62.

It is therefore preferred that component (b) comprises (a) sequence(s) being with increasing preference at least 80%, at least 85%, at least 90%, at least 95%, at least 98%, at least 99%, and 100% identical to SEQ ID NOs 60 and/or 62, or is encoded by (a) sequence(s) being with increasing preference at least 80%, at least 85%, at least 90%, at least 95%, at least 98%, at least 99%, and 100% identical to SEQ ID NOs 59 and/or 61.

Nucleotide and amino acid sequence analysis and alignment in connection with the present invention are preferably carried out using the NCBI BLAST algorithm (Stephen F. Altschul, Thomas L. Madden, Alejandro A. Schaffer, Jinghui Zhang, Zheng Zhang, Webb Miller, and David J. Lipman (1997), Nucleic Acids Res. 25:3389-3402). BLAST can be used for nucleotide sequences (nucleotide BLAST) and amino acid sequences (protein BLAST). The skilled person is aware of additional suitable programs to align nucleic acid sequences.

In accordance with another preferred embodiment of the first aspect of the invention components (a), (b) and/or (c) are fused to each other by a flexible linker, preferably a flexible peptide linker and most preferably a flexible peptide linker of at least 5 amino acids.

A peptide-linker is a short amino acid sequence, preferably in the range of 5 to 50 amino acids. The peptide linker is preferably a GS-linker. A GS-linker consists only of glycine and serine amino acids.

Preferred examples of GS-linker are selected from the seven linkers of SEQ ID NOs 13 to 17, 63 and 64 which are encoded by the nucleic acid sequences of SEQ ID NOs 18 to 22, 65 and 66.

In accordance with a further preferred embodiment of the first aspect of the invention component (a) is at the N-terminus, component (b) is between components (a) and (c), and part (c) is at the C-terminus of the fusion protein.

This particular orientation of components (a), (b) and (c) is illustrated by the examples. While the orientation of components (a), (b) and (c) is not particularly limited in particular this orientation was used in the examples and found to work well.

In accordance with a yet further preferred embodiment of the first aspect of the invention the fusion protein further comprises a purification tag, preferably a His-tag or a myc-tag.

A purification tag (or affinity tag) is appended to proteins so that they can be purified from their crude biological source using an affinity technique (such as affinity chromatography). These include chitin binding protein (CBP), maltose binding protein (MBP), Strep-tag, glutathione-S-transferase (GST), polyhistidine (His), and myc.

A His-tag or a myc-tag are preferred since they are used in the examples. A polyhistidine-tag is an amino acid motif in proteins that typically consists of at least six histidine (His) residues, often at the N- or C-terminus of the protein. A polyhistidine-tag is most commonly simply referred to as His-Tag. A myc tag is a polypeptide protein tag derived from the c-myc gene product that can be added to a protein using recombinant DNA technology.

The His-tag preferably has the amino acid sequence of SEQ ID NO: 23 or is encoded by SEQ ID NO: 24. The myc-tag preferably has the amino acid sequence of SEQ ID NO: 25 or is encoded by SEQ ID NO: 26. Also envisioned are linkers comprising or being encoded by a sequence having at least 80%, preferably at least 90% and most preferably at least 95% sequence identity with any one of SEQ ID NOS 23 to 26.

In accordance with a further preferred embodiment of the first aspect of the invention the antigens/targets of (a) 4-1BB, NKG2D, NKp30, NKp46, NKp44, 2B4, CD28 or DNAM-1, of (b) IL-15, IL-2, IL-18, IL-21 or IL-12 and/or of (c) are human antigens/targets.

The use of human targets/antigens is particularly advantageous for the expansion of human NK cells, T cells and/or NTK cells. The expansion of human NK cells, T cells and/or NTK cells is particular of interest since they can be used for NK/T/NTK cell-based therapeutic strategies, such as the treatment of cancer or in adoptive immunotherapy (see review of Rezani and Rouce (2015), Front. Immunol.)

In accordance with another preferred embodiment of the first aspect of the invention component (c) comprises IL-15 fused to the sushi domain of the IL-15 receptor.

The Sushi domain of soluble IL-15 receptor alpha is essential and sufficient for binding IL-15 (see Xq et al. (2001), J Immunol; 167(1):277-82). The sushi domain is a common motif in protein-protein interaction. Sushi domains are also known as short consensus repeats or type 1 glycoprotein motifs. They have been identified on a number of protein-binding molecules, including complement components C1r, C1s, factor H, and C2m as well as the nonimmunologic molecules factor XIII and β2-glycoprotein. A typical Sushi domain has approximately 60 aa residues and contains four cysteines. The first cysteine forms a disulfide bond with the third cysteine, and the second cysteine forms a disulfide bridge with the fourth cysteine. The two disulfide bonds are essential to maintain the tertiary structure of the protein. In the exemplified fusion protein of the invention as used in the appended examples and thus also preferably the sushi domain of the IL-15 receptor is used to connect IL-15 to the remainder components of the fusion protein.

It is known that the binding of IL-15 to the sushi domain of IL-15R inhibits inflammatory and allogenic responses. This inhibition can be advantageous for in vivo applications, for example, in order to prevent side effects.

The IL-15 herein preferably comprises the amino acid of SEQ ID NO: 27 or is encoded by SEQ ID NO: 28.

The sushi domain of IL-15R preferably comprises the amino acid of SEQ ID NO: 29 or is encoded by SEQ ID NO: 30.

Also envisioned sequences having at least 80%, preferably at least 90%, and most preferably at least 95% sequence identity with any one of SEQ ID NOs 27 to 30.

In accordance with another preferred embodiment of the first aspect of the invention component (c) comprises IL-2.

The IL-2 herein preferably comprises the amino acid of SEQ ID NO: 67 or is encoded by SEQ ID NO: 68.

Also envisioned sequences having at least 80%, preferably at least 90%, and most preferably at least 95% sequence identity with SEQ ID NO: 67 or 68.

In accordance with a further preferred embodiment of the first aspect of the invention the fusion protein comprises or consists (a) an amino acid sequence of SEQ ID NO: 31, 33, 35, 37, 69, 71, 73, 75 or 77, (b) an amino acid sequence being encoded by SEQ ID NO: 32, 34, 36, 38, 70, 72, 74, 76 or 78, (c) an amino acid sequence being with increasing preference at least 80%, at least 85%, at least 90%, at least 95%, at least 98%, at least 99%, and 100% identical to SEQ ID NO: 31, 33, 35, 37, 69, 71, 73, 75 or 77, or (d) an amino acid sequence being encoded with increasing preference by a nucleotide sequence being at least 80%, at least 85%, at least 90%, at least 95%, at least 98%, at least 99%, and 100% identical to SEQ ID NO: 32, 34, 36, 38, 70, 72, 74, 76 or 78.

SEQ ID NO: 31 is the amino acid sequence and SEQ ID NO: 32 is the nucleic acid sequence of the exemplified fusion protein RTX-CD137scFv-IL-15.

SEQ ID NO: 33 is the amino acid sequence and SEQ ID NO: 34 is the nucleic acid sequence of the exemplified fusion protein BCMA-CD137scFv-IL-15.

SEQ ID NO: 35 is the amino acid sequence and SEQ ID NO: 36 is the nucleic acid sequence of the exemplified fusion protein RTX-DuoFab-CD137scFv-IL-15.

SEQ ID NO: 37 is the amino acid sequence and SEQ ID NO: 38 is the nucleic acid sequence of the exemplified fusion protein BCMA-DuoFab-CD137scFv-IL-15.

SEQ ID NO: 69 is the amino acid sequence and SEQ ID NO: 70 is the nucleic acid sequence of the exemplified fusion protein CD19-CD137scFv-IL-15.

SEQ ID NO: 71 is the amino acid sequence and SEQ ID NO: 72 is the nucleic acid sequence of the exemplified fusion protein RTX-CD137scFvdss-IL-15.

SEQ ID NO: 73 is the amino acid sequence and SEQ ID NO: 74 is the nucleic acid sequence of the exemplified fusion protein RTX-CD137scFv-IL-2.

SEQ ID NO: 75 is the amino acid sequence and SEQ ID NO: 76 is the nucleic acid sequence of the exemplified fusion protein RTX-NKp46scFv-IL-15.

SEQ ID NO: 77 is the amino acid sequence and SEQ ID NO: 78 is the nucleic acid sequence of the exemplified fusion protein RTX-NKG2DscFv-IL-15.

The above exemplified fusion proteins comprise next to components (a) to (c) and linkers also a truncated hinge region, a CH1 region and spacer sequences.

The truncated hinge region has the amino acid sequence of SEQ ID NO: 39 and the nucleic acid sequence of SEQ ID NO: 40.

The CH1 region has the amino acid sequence of SEQ ID NO: 41 and the nucleic acid sequence of SEQ ID NO: 42.

Spacer sequence 1 has the amino acid sequence of SEQ ID NO: 43 and the nucleic acid sequence of SEQ ID NO: 44. Spacer sequence 2 has the amino acid sequence of SEQ ID NO: 45 and the nucleic acid sequence of SEQ ID NO: 46.

The present invention relates in a second aspect to a nucleic acid sequence, a set of nucleic acid molecules, an expression vector or set of expression vectors encoding the fusion protein of the first aspect.

The term “nucleic acid molecule” in accordance with the present invention includes DNA, such as cDNA or double or single stranded genomic DNA and RNA. In this regard, “DNA” (deoxyribonucleic acid) means any chain or sequence of the chemical building blocks adenine (A), guanine (G), cytosine (C) and thymine (T), called nucleotide bases, that are linked together on a deoxyribose sugar backbone. DNA can have one strand of nucleotide bases, or two complimentary strands which may form a double helix structure. It further includes RNA. “RNA” (ribonucleic acid) means any chain or sequence of the chemical building blocks adenine (A), guanine (G), cytosine (C) and uracil (U), called nucleotide bases, that are linked together on a ribose sugar backbone. RNA typically has one strand of nucleotide bases, such as mRNA. Included are also single- and double-stranded hybrid molecules, i.e., DNA-DNA, DNA-RNA and RNA-RNA. The nucleic acid molecule may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Nucleic acid molecules, in the following also referred as polynucleotides, may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Further included are nucleic acid mimicking molecules known in the art such as synthetic or semi-synthetic derivatives of DNA or RNA and mixed polymers. Such nucleic acid mimicking molecules or nucleic acid derivatives according to the invention include phosphorothioate nucleic acid, phosphoramidate nucleic acid, 2′-O-methoxyethyl ribonucleic acid, morpholino nucleic acid, hexitol nucleic acid (HNA), peptide nucleic acid (PNA) and locked nucleic acid (LNA) (see Braasch and Corey, Chem Biol 2001, 8:1). LNA is an RNA derivative in which the ribose ring is constrained by a methylene linkage between the 2′-oxygen and the 4′-carbon. Also included are nucleic acids containing modified bases, for example thio-uracil, thio-guanine and fluoro-uracil. A nucleic acid molecule typically carries genetic information, including the information used by cellular machinery to make proteins and/or polypeptides. The nucleic acid molecule of the invention may comprise promoters, enhancers, response elements, signal sequences, polyadenylation sequences, introns, 5′- and 3′-non-coding regions, and the like.

As mentioned, the nucleic acid molecule according to the invention encodes the fusion protein of the invention. The fusion protein of the invention may also be encoded by a set of nucleic acid molecules, preferably by a set of two nucleic acid molecules. This is because antibodies or fragments thereof as comprised in the fusion protein (e.g. a full-length antibody, scFv or Fab) comprise heavy and light chain sequences which, for example, upon expression in a cell, self-assemble into an antibody. The heavy and light chain sequences can be encoded by a set of different nucleic acid molecules, preferably by two nucleic acid molecules.

The term “vector” in accordance with the invention means preferably a plasmid, cosmid, virus, bacteriophage or another vector used e.g. conventionally in genetic engineering which encoding the fusion protein of the invention in expressible form. For the same reasons as discussed in connection with the set of nucleic acid molecules of the invention, the fusion protein of the invention may also be encoded by a set of vectors, preferably by a set of two vectors.

The nucleic acid molecule(s) encoding the fusion protein of the invention may, for example, be inserted into several commercially available vectors. Non-limiting examples include prokaryotic plasmid vectors, such as of the pUC-series, pBluescript (Stratagene), the pET-series of expression vectors (Novagen) or PCRTOPO (Invitrogen) and vectors compatible with an expression in mammalian cells like pREP (Invitrogen), pSec Tag2 (Invitrogen), pcDNA3 (Invitrogen), pCEP4 (Invitrogen), pMC1neo (Stratagene), pXT1 (Stratagene), pSG5 (Stratagene), EBO-pSV2neo, pBPV-1, pdBPVMMTneo, pRSVgpt, pRSVneo, pSV2-dhfr, plZD35, pLXIN, pSIR (Clontech), pIRES-EGFP (Clontech), PEAK-10 (Edge Biosystems) pTriEx-Hygro (Novagen) and pCINeo (Promega). Examples for plasmid vectors suitable for Pichia pastoris comprise e.g. the plasmids pAO815, pPIC9K and pPIC3.5K (all Invitrogen).

The nucleic acid molecules inserted into the vector can e.g. be synthesized by standard methods, or isolated from natural sources. Ligation of the coding sequences to transcriptional regulatory elements and/or to other amino acid encoding sequences can also be carried out using established methods. Transcriptional regulatory elements (parts of an expression cassette) ensuring expression in prokaryotes or eukaryotic cells are well known to those skilled in the art. These elements comprise regulatory sequences ensuring the initiation of transcription (e. g., translation initiation codon, promoters, such as naturally-associated or heterologous promoters and/or insulators; see above), internal ribosomal entry sites (IRES) (Owens, Proc. Natl. Acad. Sci. USA 98 (2001), 1471-1476) and optionally poly-A signals ensuring termination of transcription and stabilization of the transcript. Additional regulatory elements may include transcriptional as well as translational enhancers. Preferably, the polynucleotide(s) encoding the fusion protein of the invention is operatively linked to such expression control sequences allowing expression in prokaryotes or eukaryotic cells. The vector may further comprise nucleic acid sequences encoding secretion signals as further regulatory elements. Such sequences are well known to the person skilled in the art. Furthermore, depending on the expression system used, leader sequences capable of directing the expressed polypeptide to a cellular compartment may be added to the coding sequence of the polynucleotide of the invention. Such leader sequences are well known in the art.

Furthermore, it is preferred that the vector comprises a selectable marker. Examples of selectable markers include genes encoding resistance to neomycin, ampicillin, hygromycine, and kanamycin. Specifically designed vectors allow the shuttling of DNA between different hosts, such as bacteria-fungal cells or bacteria-animal cells (e. g. the Gateway system available at Invitrogen). An expression vector according to this invention is capable of directing the replication, and the expression, of the polynucleotide and encoded peptide or fusion protein of this invention. Apart from introduction via vectors such as phage vectors or viral vectors (e.g. adenoviral, retroviral), the nucleic acid molecules as described herein above may be designed for direct introduction or for introduction via liposomes into a cell. Additionally, baculoviral systems or systems based on vaccinia virus or Semliki Forest virus can be used as eukaryotic expression systems for the nucleic acid molecules of the invention.

The present invention relates in a third aspect to a host cell, preferably a non-human host cell comprising the nucleic acid molecule or the expression vector of the second aspect.

The term “host cell” means any cell of any organism that is selected, modified, transformed, grown, or used or manipulated in any way, for the production of the fusion protein of the invention by the cell. The host cell is therefore generally an ex vivo or in vitro cell and/or an isolated cell.

The host cell of the invention is typically produced by introducing the nucleic acid molecule(s) or vector(s) of the invention into the host cell which upon its/their presence mediates the expression of the nucleic acid molecule(s) of the invention encoding the fusion protein of the invention. The host from which the host cell is derived or isolated may be any prokaryote or eukaryotic cell or organism, preferably with the exception of human embryonic stem cells that have been derived directly by destruction of a human embryo.

Suitable prokaryotes (bacteria) useful as hosts for the invention are, for example, those generally used for cloning and/or expression like E. coli (e.g., E coli strains BL21, HB101, DH5a, XL1 Blue, Y1090 and JM101), Salmonella typhimurium, Serratia marcescens, Burkholderia glumae, Pseudomonas putida, Pseudomonas fluorescens, Pseudomonas stutzeri, Streptomyces lividans, Lactococcus lactis, Mycobacterium smegmatis, Streptomyces coelicolor or Bacillus subtilis. Appropriate culture mediums and conditions for the above-described host cells are well known in the art.

A suitable eukaryotic host cell may be a vertebrate cell, an insect cell, a fungal/yeast cell, a nematode cell or a plant cell. The fungal/yeast cell may a Saccharomyces cerevisiae cell, Pichia pastoris cell or an Aspergillus cell. Preferred examples of a host cell to be genetically engineered with the nucleic acid molecule or the vector(s) of the invention are cells of yeast, E. coli and/or a species of the genus Bacillus (e.g., B. subtilis). In one preferred embodiment the host cell is a yeast cell (e.g. S. cerevisiae).

In a different preferred embodiment the host cell is a mammalian host cell, such as a Chinese Hamster Ovary (CHO) cell, mouse myeloma lymphoblastoid, human embryonic kidney cell (HEK-293), human embryonic retinal cell (Crucell's Per.C6), or human amniocyte cell (Glycotope and CEVEC). The cells are frequently used in the art to produce recombinant proteins. CHO cells are the most commonly used mammalian host cells for industrial production of recombinant protein therapeutics for humans.

The present invention also relates to a transgenic animal, preferably a non-human transgenic animal comprising the vector of the invention.

Transgenic animals can be used for the production of antibodies as is reviewed, for example, in Brüggemann (2015), Arch Immunol Ther Exp (Warsz); 63(2): 101-108. The transgenic animal is preferably a mammal other than human. The antibodies may also be produced such that the antibodies can be obtained from the milk of transgenic mammals. The mammal is therefore preferably a goat, sheep or cow.

The present invention relates in a fourth aspect to a method for producing the fusion protein of the first aspect comprising (a) culturing the host cell of the third aspect under conditions where the host cell expresses the fusion protein of the first aspect, and (b) isolating the fusion protein of the first aspect as expressed in (a).

The term “culturing” specifies the process by which host cells are grown under controlled conditions. These conditions may vary dependent on the host cell used. The skilled person is well aware of methods for establishing optimized culturing conditions. Moreover, methods for establishing, maintaining and manipulating a cell culture have been extensively described in the state of the art.

Methods of isolation of the fusion protein of the invention are well-known in the art and comprise without limitation method steps such as ion exchange chromatography, gel filtration chromatography (size exclusion chromatography), affinity chromatography, high pressure liquid chromatography (HPLC), reversed phase HPLC, disc gel electrophoresis or immunoprecipitation, see, for example, Antibody Purification Handbook, GE Healthcare, 18-1037-46.

The fusion protein of the invention as expressed in (a) in accordance with the invention refers to the product of a process implying, that in the host cell a process can be induced by which information from nucleic acid molecule(s) encoding the fusion protein of the invention is/are used in the synthesis of the fusion protein of the invention. Several steps in this process may be modulated, including the transcription, RNA splicing, translation, and post-translational modification of the fusion protein of the invention by methods know in the art. Accordingly, such modulation may allow for control of the timing, location, and amount of fusion protein produced.

The present invention relates in a fifth aspect to a composition, preferably a pharmaceutical composition or a kit comprising the fusion protein, the nucleic acid molecule, set of nucleic acid molecules, expression vector, set of expression vectors, or the host cell of the above aspects of the invention.

The composition of the invention comprises the fusion protein, the nucleic acid molecule, set of nucleic acid molecules, expression vector or set of expression vectors, or the host cell of the above aspects of the invention and preferably at least one further component, such as a solvent, carrier or excipient.

In accordance with the present invention, the term “pharmaceutical composition” relates to a composition for administration to a patient, preferably a human patient. The pharmaceutical composition of the invention comprises the fusion protein, the nucleic acid molecule, set of nucleic acid molecules, expression vector or set of expression vectors, or the host cell of the above aspects of the invention. It may, optionally, comprise further molecules capable of altering the characteristics of the compounds of the invention thereby, for example, stabilizing, modulating and/or activating their function. The composition may be in solid, liquid or gaseous form and may be, inter alia, in the form of (a) powder(s), (a) tablet(s), (a) solution(s) or (an) aerosol(s). The pharmaceutical composition of the present invention may, optionally and additionally, comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, organic solvents including DMSO etc. Compositions comprising such carriers can be formulated by well-known conventional methods. These pharmaceutical compositions can be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. The therapeutically effective amount for a given situation will readily be determined by routine experimentation and is within the skills and judgement of the ordinary clinician or physician. Generally, the regimen as a regular administration of the pharmaceutical composition should be in the range of 1 μg to 5 g units per day. However, a more preferred dosage might be in the range of 0.0001 mg to 100 mg/kg bodyweight, even more preferably 0.01 mg to 50 mg/kg bodyweight and most preferably 20 mg to 50 mg/kg bodyweight per day. The length of treatment needed to observe changes and the interval following treatment for responses to occur vary depending on the desired effect. The particular amounts may be determined by conventional tests, which are well known to the person skilled in the art.

The composition of the invention comprises the fusion protein, the nucleic acid molecule, set of nucleic acid molecules, expression vector or set of expression vectors, or the host cell of the above aspects of the invention and preferably instructions how to use the kit; i.e., for instance, how to use the fusion protein for expanding NK cells, T cells and/or NTK cells.

The present invention relates in a sixth aspect to the fusion protein, the nucleic acid molecule, set of nucleic acid molecules, expression vector or set of expression vectors, or the host cell of the above aspects of the invention in optionally in combination with CAR NK-cells or CAR T-cells for use in the treatment of a tumor.

Upon the administration of the fusion protein, the nucleic acid molecule, set of nucleic acid molecules, expression vector or set of expression vectors, or the host cell of the above aspects of the invention to a subject, the NK cells, T cells and/or NTK cells in the subject are expanded. In connection with this aspect it is preferred that component (a) is an antibody or an antibody fragment binding to an antigen being expressed on the surface of a target cell of NK cells, T cells and/or NTK cells that is a tumor cell. More preferably the antigen can be found on the surface of the cells of tumor within the subject to be treated. The subject is with increasing preference a mammal, primate and human.

An autologous CAR (chimeric antigen receptor)-NK cell therapy or CAR T-cell therapy comprises several steps. First, NK cells or T cells are isolated from patient's or donor's blood. Subsequently, the NK cells or T cells are transduced with CAR-encoding genes using (mostly) viral vectors. CAR-NK or T cells are expanded until sufficient cell numbers are attained and are adoptively transferred into the patient to fight malignant cells. Prior to infusion of the CAR-NK or T cells, lymphodepletion is generally performed in most therapeutic settings to allow efficient cell engraftment. It is important to mention that CAR-NK or T cells offer the potential to be an “off-the-shelf” product.

The present invention relates in a seventh aspect to a method for the ex vivo or in vitro expansion of NK-cells, T cells and/or NTK cells comprising (a) coculturing NK-cells, T cells and/or NTK cells with target cells of NK cells, T cells and/or NTK cells, preferably B cells or tumor cells in the presence of the fusion protein, the nucleic acid sequence, set of nucleic acid sequences, expression vector set of expression vectors, or the host cell of the above aspects of the invention, (b) optionally purifying or isolating the expanded NK-cells, T cells and/or NTK cells as obtained in step (a) from the coculture.

In accordance with a preferred embodiment of the seventh aspect the NK cells, T-cells and/or NTK cells are purified NK cells, T cells and/or NTK cells, are comprised in PBMCs, derived from iPSCs, or are CAR NK or T-cells.

As discussed above, during CAR-NK or T cell therapy NK or T cells, such as purified NK or T cells, NK or T cells comprised in PBMCs or CAR NK or T-cells need to be expanded ex vivo or in vitro before they are administered to the subject to be treated, such as a subject having a tumor. The method of the seventh aspect is particularly well suited for such an expansion of the NK cells, T cells and/or NTK cells. As discussed above, the appended examples show that expansion rates between 10-10,000-fold were achieved by the fusion protein of the invention.

In accordance with a further preferred embodiment of the seventh aspect the NK cells, T-cells and/or NTK may be pre-cultured (e.g. 12-24 h) prior to step (a) with IL-12, IL-15 and/or IL-18. This preculturing step in polarizes the cells towards a memory-like phenotype.

Regarding the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The above considerations apply mutatis mutandis to all appended claims.

The figures show.

FIG. 1. Design, concept, purification, and biochemical characterization of the fusion proteins. (A) Scheme of the structure of the RTX-CD137scFv-IL-15 fusion; CD20-VH, CD20-VL from the CD20 antibody rituximab, cDNA sequence coding for immunoglobulin heavy and light chain constant region; CD137 VL, CD137 VH, cDNA sequences coding for the variable heavy and light chain constant regions building a scFv with specificity for CD137 (4-1BB); sushi domain, hIL-15, cDNA sequence coding for sushi domain and human interleukin 15; (B) Mechanism of action (C) Evaluation of the purity and integrity of the fusion proteins by SDS-PAGE and Coomassie staining. Reduced and non-reduced protein samples. (D-E) Specific antigen binding of fusion proteins. Dose-dependent binding of the fusion proteins (RTX-CD137scFv-IL-15—red filled circle; RTX-CD137scFv—blue filled square; RTX-IL-15—lilac filled diamond; 4D5-CD137scFv-IL-15—black circle; IL-15—black triangle; trastuzumab—black filled triangle) to CD20-positive Ramos cells (D) and 4-1BB (CD137) positive CRRF-CEM cells (E). The mean fluorescence values at saturating concentrations for each cell line were set to 100% and all other experimental values were normalized to this value. Data are presented as mean values of three independent measurements with error bars representing ±SEM, *=<0.05 (F) To determine the functionality of IL-15 of the different antibody derivatives (RTX-CD137scFv-IL-15—red filled circle; RTX-CD137scFv—blue filled square; RTX-IL-15—lilac filled square; 4D5-CD137scFv-IL-15—black circle; IL-15—black filled triangle), serial dilutions of the fusion proteins were incubated with CTLL-2 cells. After 48 h the MTT reagent was added. The mean fluorescence values at saturating concentrations for IL-15 concentration were set to 100% and all other experimental values were normalized to this value. Data are presented as mean values of four independent measurements with error bars representing ±SEM; *=<0.05.

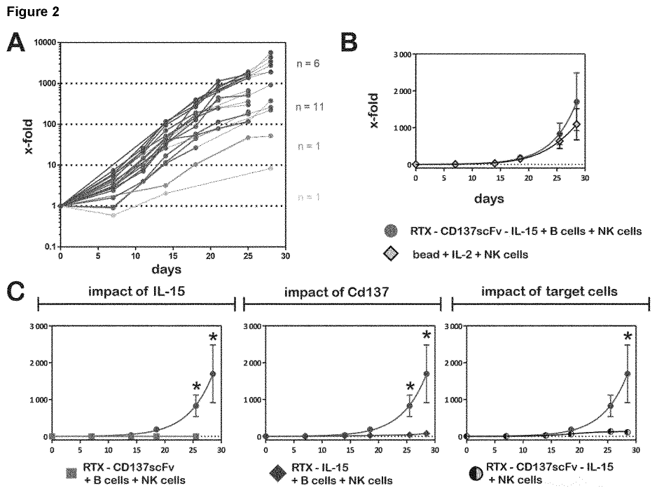

FIG. 2. Expansion capability of the fusion proteins. (A) Expansion rates of 19 independent NK cell expansions (B) To compare the expansion capability of the fusion protein carrying all structural components and a commercial expansion kit, freshly isolated NK cells from healthy donors were co-incubated with RTX-CD137scFv-IL-15 and B cells. RTX-CD137scFv-IL-15+B cells—red filled circle and the expansion beads+IL-2 (bead+IL-2—grey diamond) (C) To determine the impact of the different structural components and the impact of target cell presence, freshly isolated NK cells from healthy donors were co-incubated with fusion proteins and B cells (RTX-CD137-IL-15+B cells—red filled circle; RTX-CD137scFv—blue filled square; RTX-IL-15—lilac filled square; RTX-CD137scFv-IL-15—black/grey circle). The cells were cultivated for up to 4 weeks. Every three to four days the NK cell number was determined and fresh media containing the respective fusion protein were added. The x-fold expansion of NK cells was plotted against the time in days. Data are presented as mean values of three independent measurements with error bars representing ±SEM, *=<0.05.

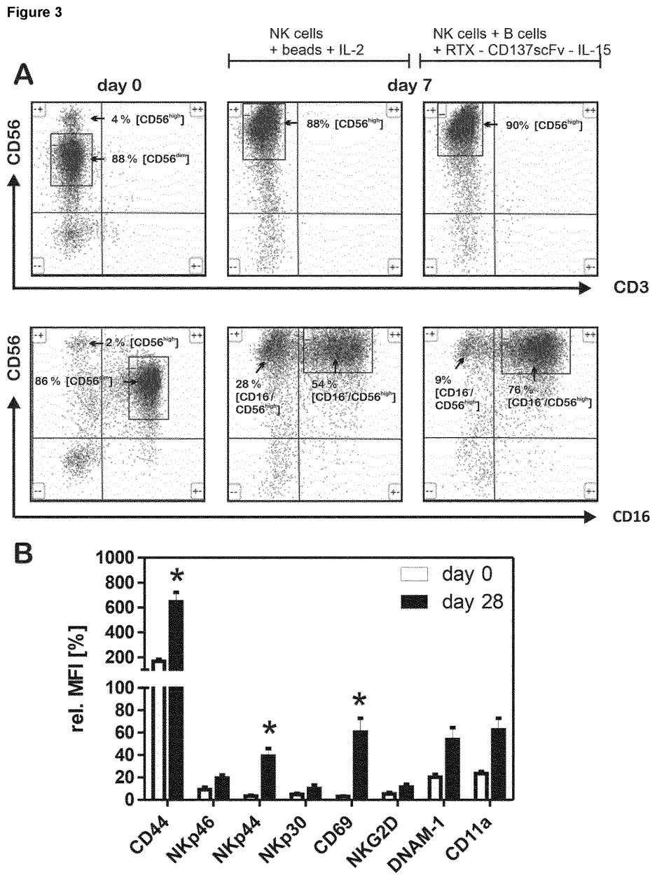

FIG. 3. Characterization of in vitro expanded NK cells from healthy donors. (A) An important role in antibody-dependent cell-mediated cytotoxicity plays the Fc receptor FcγRIIIa (CD16a). NK cells (CD56+, CD3−) express different amounts of Fc receptor FcγRIIIa (CD16a) depending on their activation status. To assess this expression flow cytometer analyses were performed with commercially purchased CD16, CD56 and CD3 antibodies at day 0 and 7. Data show representative pictures of three independent measurements. (B) The expression of certain NK cell marker (CD44; NKp46; NKp44; NKp30; CD69; NKG2D; DNAM-1; CD11a) was determined to characterize the NK cell status. The expression was determined with flow cytometer analyses at the beginning and at the end of the expansion period. Data are presented as mean values of three independent measurements with error bars representing ±SEM, *=<0.05.

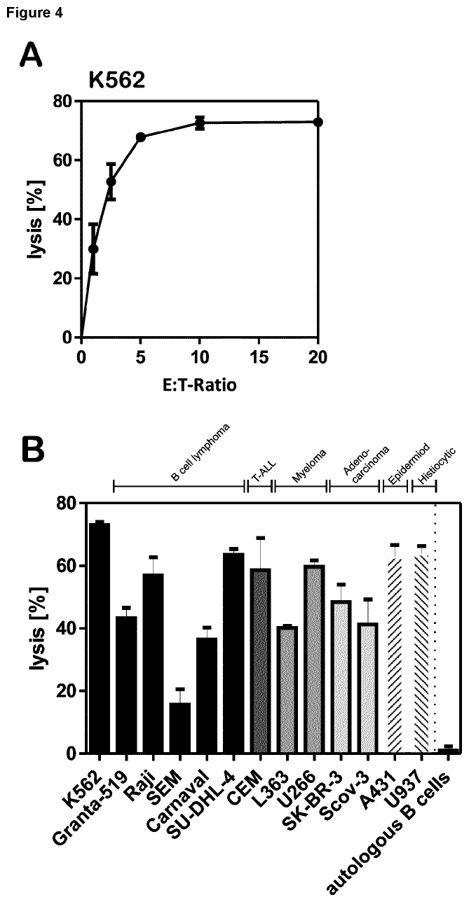

FIG. 4. Natural cytotoxicity of expanded NK cells against tumor cells and non-malignant B cells. Natural cytotoxicity of the NK cells that were expanded with the recombinant fusion proteins was determined by measuring the NK cell-mediated tumor cell lysis. 4 hr 51Cr release assays were performed with expanded NK cells as effector cells and different tumor cell lines as target cells without the addition of antibodies. (A) NK cell-dependent lysis of K562 cells. Expanded NK cells were used as effector cells at different effector to target cell (E:T) ratios. Data represent mean values +/−SEM of three NK cell donors. (B) ⁵¹Cr release assays were performed with tumor cells representing different tumor entities or non-malignant B cells. NK cells were used at a fixed E:T ratio of 10:1. Data presented are mean values +/−SEM of three NK cell donors.

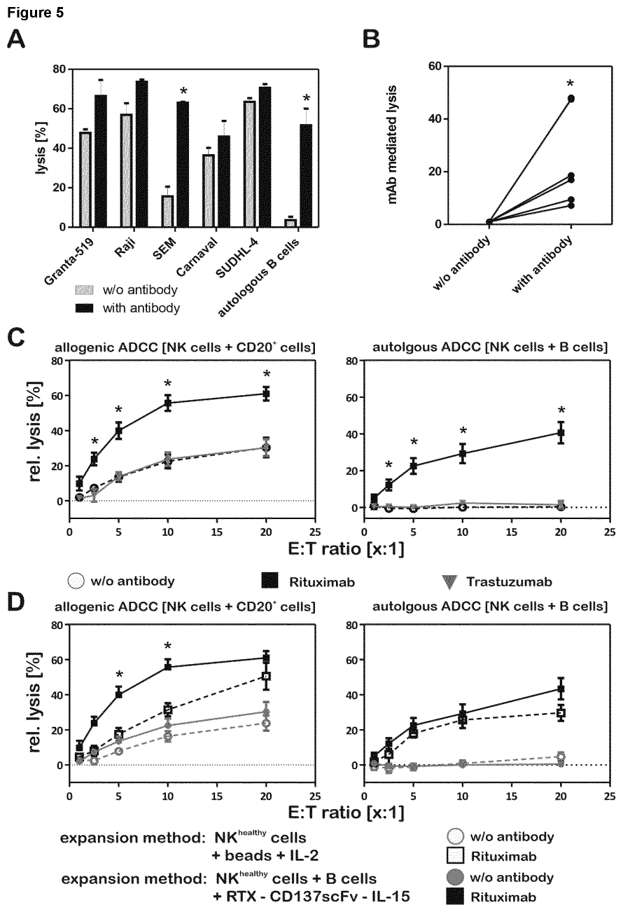

FIG. 5. Antibody-dependent cell-mediated cytotoxicity of expanded NK cells against tumor cells and non malignant B cells. (A) Comparison of natural and antibody-dependent cell-mediated cytotoxicity of NK cells that were expanded with the recombinant fusion protein. Tumor cell lysis was determined by carrying out a 4 hr 51Cr release assay with expanded NK cells as effector cells and different cell lines of B cell malignancies (Granta-519, Raji, SEM, Carnaval, SUDHL-4) or non-malignant B cells (autologous) as target cells with or without the addition of 5 μg/ml antibodies. E:T ratio 10:1, (B) The effect of the antibody-mediated tumor cell lysis was determined by carrying out a 4 hr 51Cr release assay with expanded NK cells as effector cells and different cell lines of B cell malignancies (Granta-519, Raji, SEM, Carnaval, SUDHL-4) as target cells with or without the addition of 5 μg/ml antibodies. E:T ratio 10:1. (C) Cell lysis of CD20⁺ tumor cells (allogenic) or autologous non-malignant B cells as effector cells with NK cells that were expanded with the recombinant fusion protein as effector cells at different effector to target (E:T) cell ratios. ADCC was measured in a standard 4 h chromium release assay with addition of 1 μg/ml of the respective antibodies. (D) Comparison of the ADCC efficacy of differently expanded NK cells. NK cells were either expanded with micro beads+IL-2 or with the recombinant fusion protein (RTX-CD137scFv-IL-15) and were used as effector cells at different E:T ratios in a 4 hr 51Cr release assay with 1 μg/ml of the respective antibody. Data represent mean values +/−SEM of three NK cell donors.

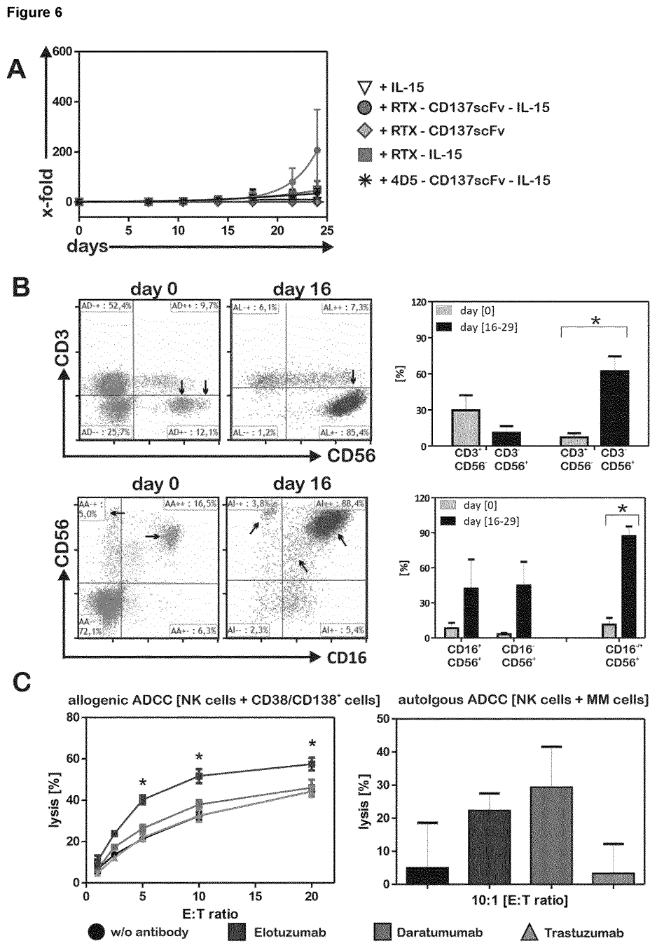

FIG. 6. Expansion of NK cells from Multiple Myeloma patients (A) To determine the expansion capability of the different fusion proteins, mononuclear cells from BM aspirates/peripheral blood of multiple myeloma (MM) patients were co-incubated with fusion proteins containing all structural components or fusion proteins missing one component. The cells were cultivated for up to 4 weeks. Every three to four days the cell number was determined and fresh media containing the respective fusion protein were added. (B) Characterization of in vitro expanded NK cells from BM aspirates/peripheral blood of multiple myeloma (MM) patients. To assess the expression status of the NK cells that were expanded with the recombinant fusion protein out of BM aspirates/peripheral blood of multiple myeloma (MM) patients flow cytometer analyses were performed with commercially purchased CD16, CD56 and CD3 antibodies at day 0 and 16. Histograms of one exemplified flow cytometric analyses and quantitative values ±SEM of three independent experiments are shown. (C) ADCC experiments with a multiple myeloma cell line (n=3, allogenic) or primary tumor cells from BM aspirates/peripheral blood of a multiple myeloma (MM) patient (exemplary, autologous) as target cells. NK cells that were expanded with the recombinant fusion protein from BM aspirates/peripheral blood of multiple myeloma (MM) patients were used as effector cells at different effector to target (E:T) cell ratios (allogenic) or at a E:T ratio of 10:1 (autologous). ADCC was measured in a standard 4 h chromium release assay with addition of 5 μg/ml of the respective antibodies.

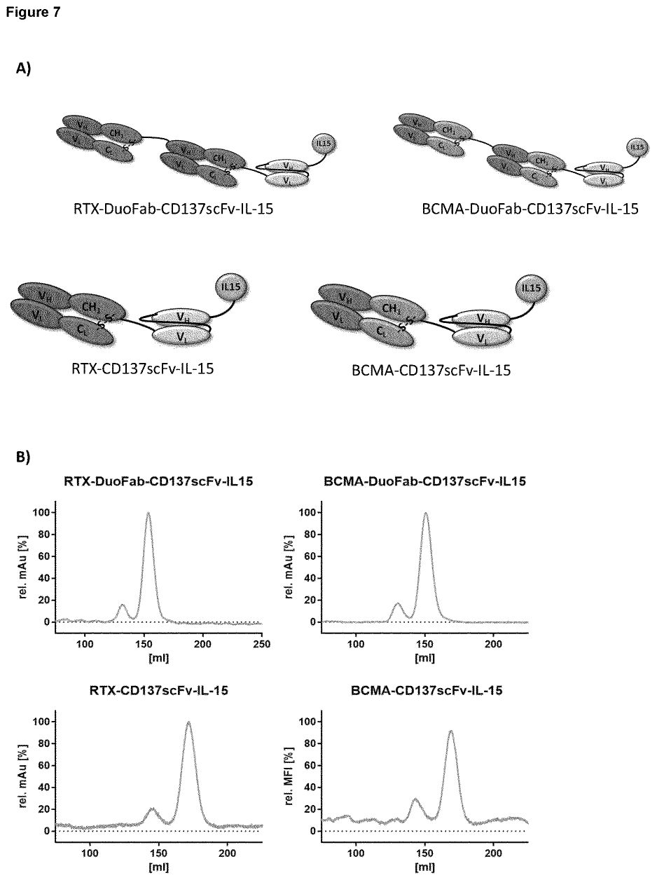

FIG. 7. Design and size exclusion chromatography analysis of monovalent and bivalent antibody derivatives. (A) schematic representation of monovalent and bivalent fusion proteins targeting CD20 or BCMA. (B) Size exclusion chromatography of purified proteins was performed by using the AKTA pure 25 liquid chromatography system. The relative protein absorbance (in milli absorbance units, mAU) was plotted against the elution volume (ml). Eluted monomers are marked by boxes.

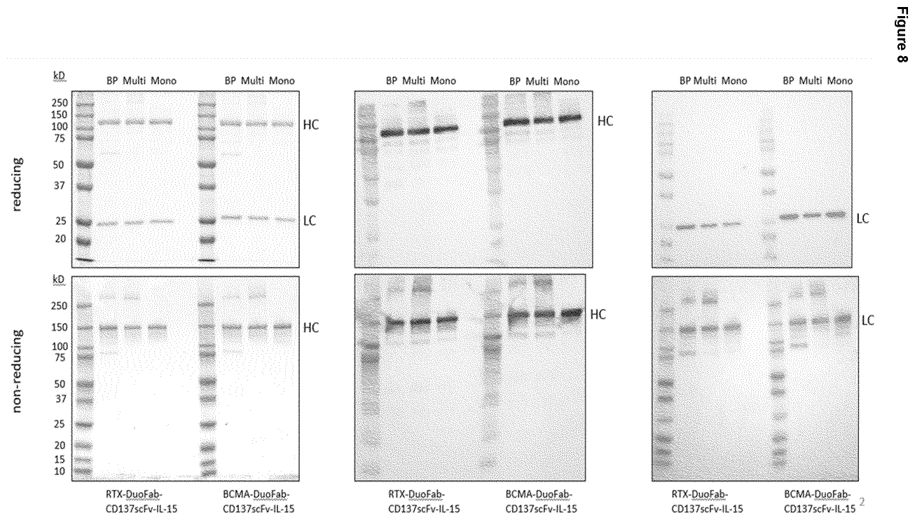

FIG. 8. Analysis of molecular mass and purity of the bivalent antibody derivatives by SDS-PAGE, Coomassie Blue staining and western blot. The purity and molecular mass of the isolated monomers and multimers were determined by SDS-PAGE and Coomassie Blue staining or western blot. Collected protein fractions before size exclusion chromatography served as controls (BP). 3 μg of the eluted protein fractions, isolated monomers and multimers were applied to SDS-Page analysis. For western blot analyses, the heavy chain derivative was detected with a polyhistidine-tag specific antibody. For the light chain an antibody directed against the kappa constant region was used. BP: before purification, HC: heavy chain derivative, LC: light chain, Mono: monomers, Multi: multimers, kDa: kilo Dalton.

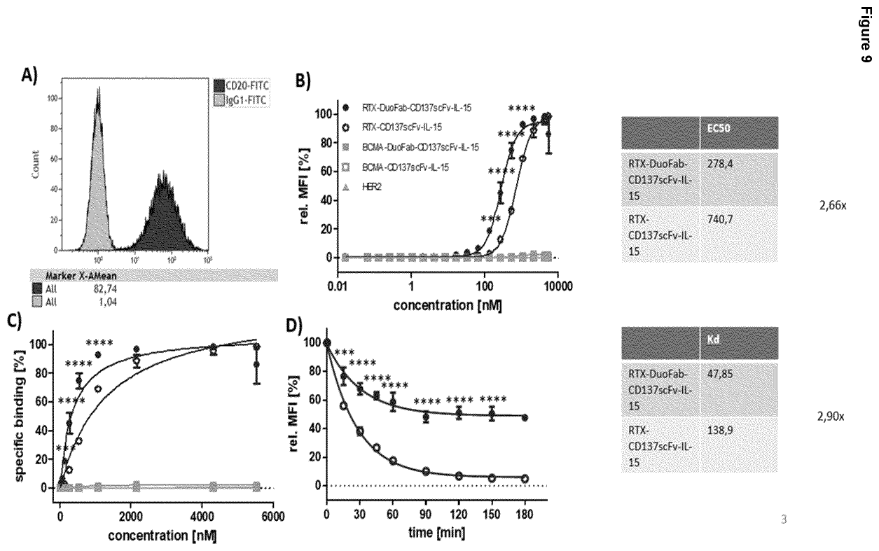

FIG. 9. CD20 binding and surface retention. To determine the binding ability of RTX-DuoFab-CD137scFv-IL-15 and RTX-CD137scFv-IL-15 flow cytometry analyses were performed using Granta-519 cells. (A) CD20 expression on Granta-519 was confirmed. The expression level was determined with an CD20 antibody. (B+C) Dose-dependent binding of the RTX-antibody derivatives was analyzed by flow cytometry and EC₅₀ values were calculated. The highest determined relative mean fluorescence intensity (t0 value for cell surface retention) was set to 100% and all other values were normalized to this point. The adapted determined relative mean fluorescence intensity (rel. MFI in %) was plotted against the protein concentration (in nM). (D) In a cell surface retention assay, dissociated molecules in the supernatant were removed at different time points. The remaining molecules on the cell surface were determined by flow cytometry. N=3. Values represent mean values +/−SEM.

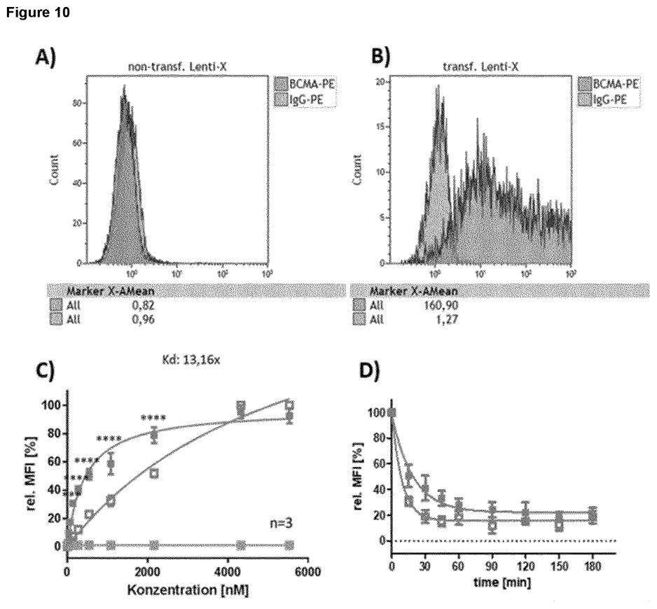

FIG. 10. Binding of BCMA and surface retention. To determine the binding ability of the BCMA-DuoFab-CD137scFv-IL-15 and the BCMA-CD137scFv-IL-15 molecules flow cytometry analyses were performed using transfected as well as non-transfected Lenti-X cells. (A+B) BCMA expression on transfected and non-transfected cells was analyzed. (C) To determine the binding activity of the BCMA-antibody derivatives flow cytometric analyses were performed. The highest determined relative mean fluorescence intensity (t0 value for cell surface retention) was set to 100% and all other values were normalized to this point. The adapted determined relative mean fluorescence intensity (rel. MFI in %) was plotted against the protein concentration (in nM). The results were displayed in a hyperbole curve. n=3. Values represent mean values +/−SEM. (D) in a cell surface retention assay dissociated molecules in the supernatant were removed at different time points. The remaining molecules on the cell surface were determined by flow cytometry. n=1.

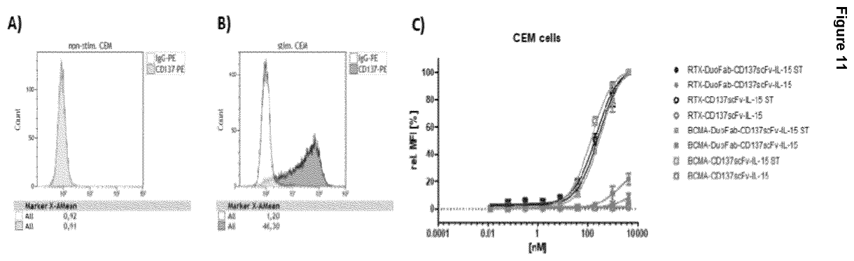

FIG. 11. CD137 binding analyses. To determine the CD137 binding ability of the antibody derivatives, flow cytometry analyses were performed using stimulated (PMA and ionomycin) as well as non-stimulated CEM cells. (A+B) Before performing the binding analyses the CD137 expression on stimulated and non-stimulated cells was analyzed. isotype control (white), CD137-antibody (grey). (C) Dose-dependent binding of the antibody derivatives was analyzed. The normalized relative mean fluorescence intensity (MFI in %) was plotted against the protein concentration (in nM). The results were displayed in a dose-response curve. n=3. Values represent mean values +/−SEM.

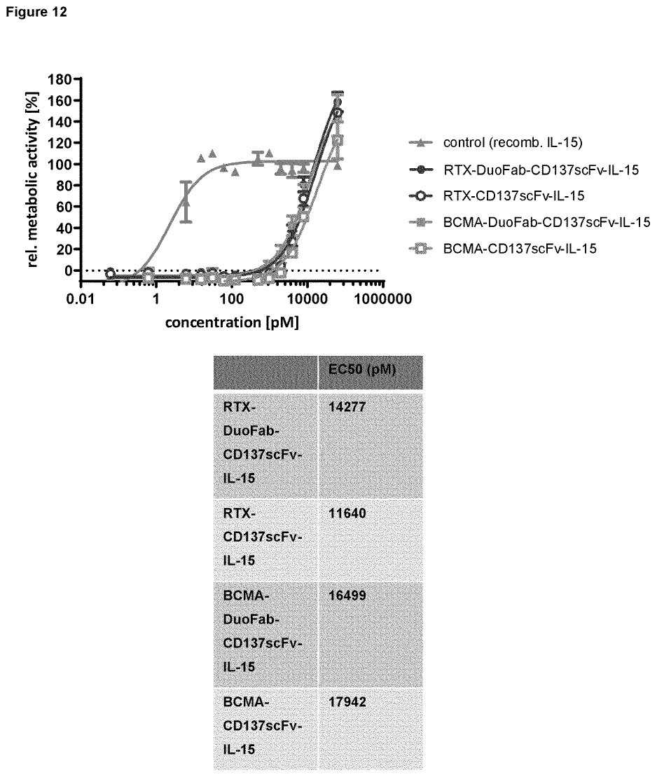

FIG. 12. Functionality of the IL-15 component of the antibody derivatives. To determine the IL-15 functionality of the different antibody derivatives, serial dilutions were prepared and incubated with the IL-15 responsive cell line CTLL2. 48 hours later, MTT reagent was added. After 24 hrs the absorbance of the purple formazan solution was measured. The relative metabolic activity (in %) was plotted against the concentration in (pM). n=3. Values represent mean values +/−SEM.

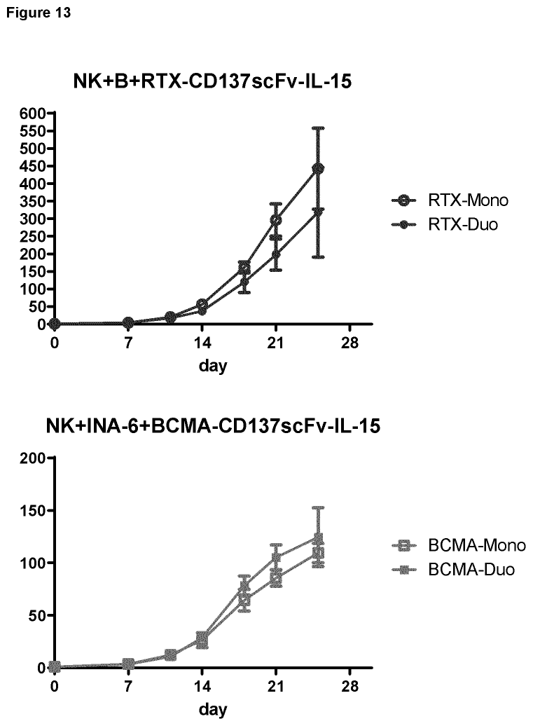

FIG. 13. Comparison of the capacity of divalent and monovalent molecules in triggering NK cell expansion. To determine the (target) cell-dependent co-activation of the divalent vs monovalent antibody derivatives, freshly isolated NK cells were co-incubated with either B cells (CD20+) and RTX antibody derivatives or INA-6 cells (BCMA+) and BCMA antibody derivatives at various concentrations. The co-cultures were cultivated for 26 days in 24-well plates. Every three to four days the NK cell number was determined and fresh media as well as antibody derivatives were added. The x-fold expansion of the NK cells was plotted against the time in days. n=3

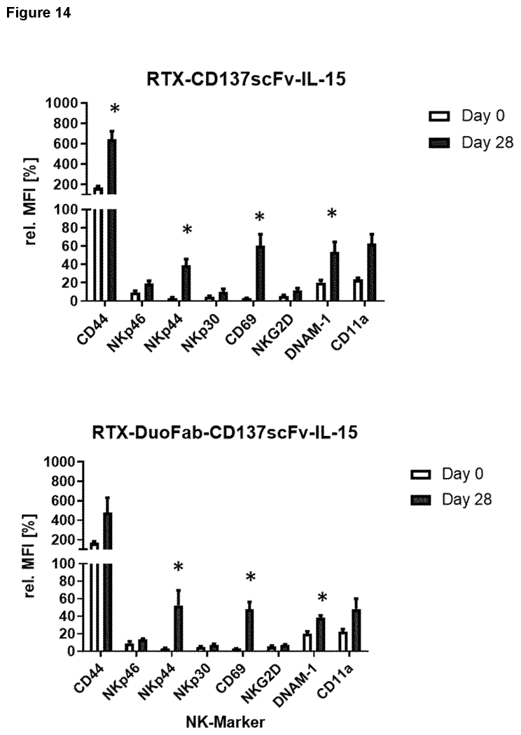

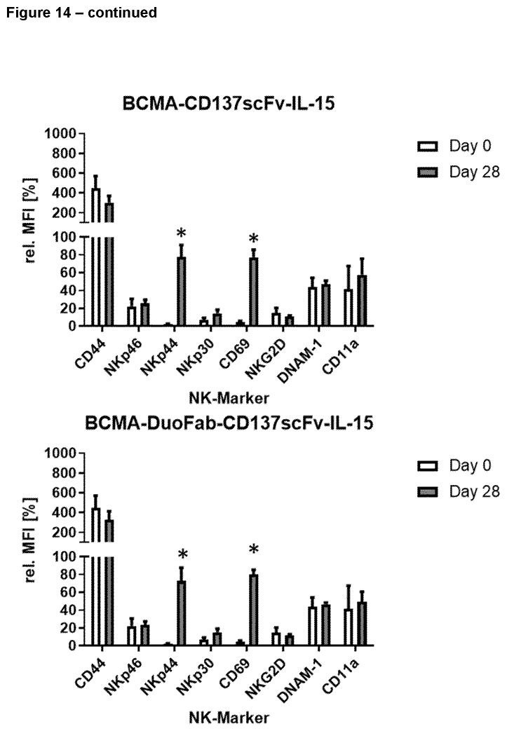

FIG. 14. Cell surface marker expression of NK cells that were expanded with antibody derivatives. Different surface markers were chosen that are linked to an activated phenotype. The expression was determined at the beginning and at the end of the expansion period. n=3

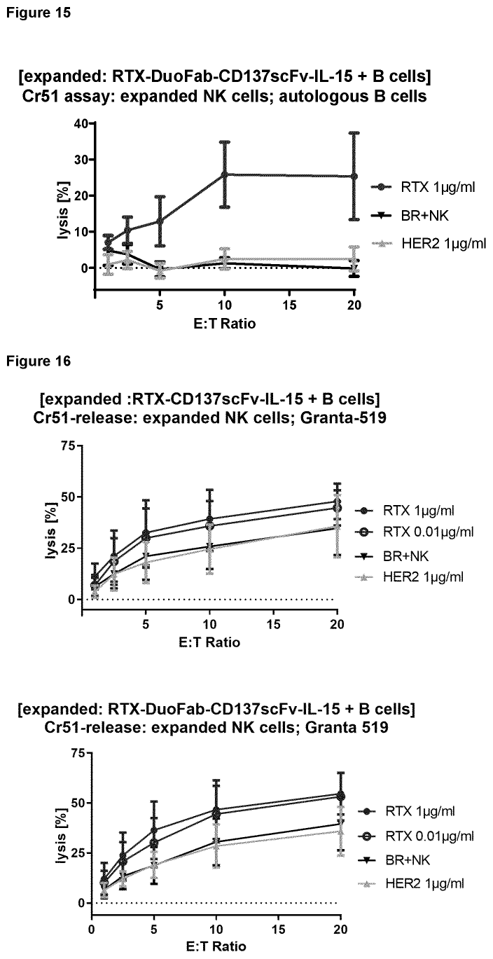

FIG. 15. NK cells expanded with RTX-DuoFab-CD137scFv-IL-15 are not cytotoxic against healthy B cells but mediate ADCC. To determine whether the expanded NK cells were still able to differentiate between healthy and malignant cells, the NK cell-mediated target cell lysis was analyzed by performing Cr51 release assays with autologous B cells as target cells. n=3.

FIG. 16. Cytotoxic activity of expanded NK cells—allogeneic ADCC setting with RTX antibody derivatives. To determine the cytotoxic activity of the expanded NK cells, the NK cell-mediated tumor cell lysis was measured by performing Cr51-release assays. Natural cytotoxicity was analyzed and ADCC reactions were performed with expanded NK cells as effector cells and Granta519 cells as target cells. Two different concentrations of the monoclonal antibody Rituximab (RTX 1 μg/ml and RTX 0.01 μg/ml) were used. Reactions that contained either no added antibodies (BR+NK=natural cytotoxicity) or the nonrelevant antibody control Trastuzumab (HER2) served as controls. n=3.

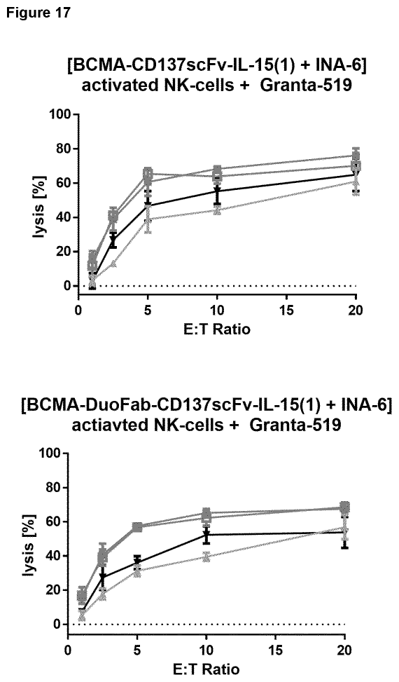

FIG. 17. Cytotoxic activity of expanded NK cells—allogeneic setting with BCMA antibody derivatives. To determine the cytotoxic activity of the expanded NK cells, the NK cell-mediated tumor cell lysis was measured by performing Cr51-release assays. Natural cytotoxicity was analyzed and ADCC reactions were performed with expanded NK cells as effector cells and Granta519 cells as target cells. Two different concentrations of the monoclonal antibody Rituximab (RTX 1 μg/ml and RTX 0.01 μg/ml) were used. Reactions that contained either no added antibodies (BR+NK=natural cytotoxicity) or the nonrelevant antibody Trastuzumab (HER2) served as controls. n=3.

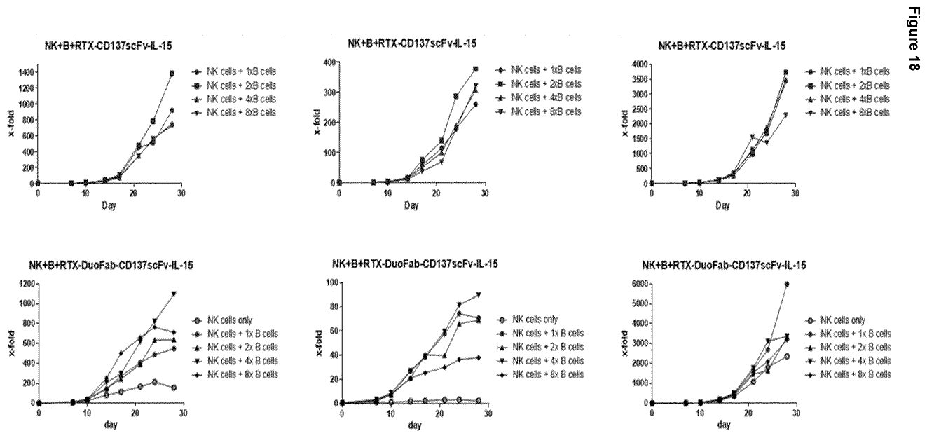

FIG. 18. Impact of frequency of B cell stimulation on NK cell expansion. Quantification of ex vivo NK cell expansion to determine the (tumor) cell-dependent co-activation of the antibody derivatives. Freshly isolated NK cells were co-incubated with the expansion molecule RTX-CD137scFv-IL-15 or RTX-DuoFab-CD137scFv-IL-15 and B cells (CD20+). The co-cultures were cultivated for 28 days. Every three to four days the NK cell number was determined and fresh media as well as antibody was added. The B cells were added at different time points (once at the beginning, twice (day 0 and day 14), once a week or twice a week). The x-fold expansion of the NK cells was plotted against the time in days. n=3

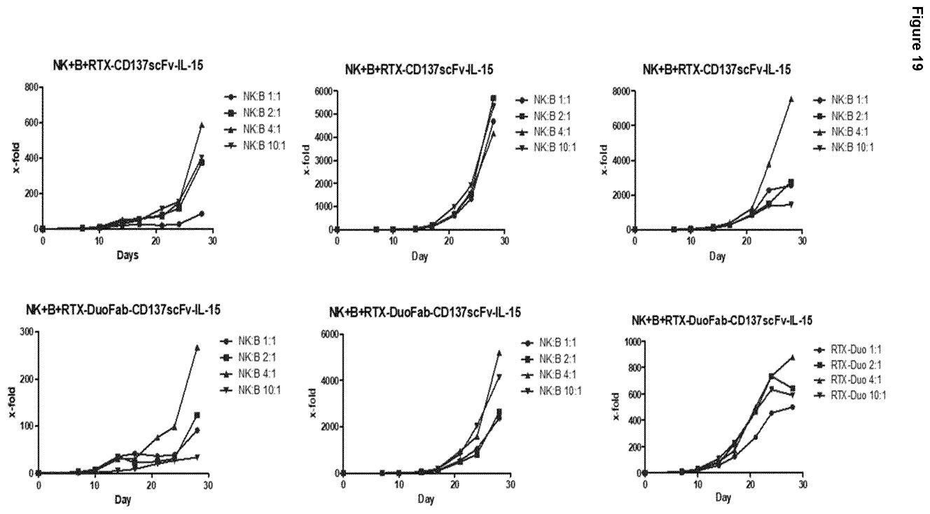

FIG. 19. Impact of B cell-to-NK cell ratio on NK cell expansion. Quantification of ex vivo NK cell expansion to determine the (target) cell-dependent co-activation of the RTX-CD137scFv-IL-15 or RTX-DuoFab-CD137scFv-IL-15. Freshly isolated NK cells were co-incubated with the expansion molecules RTX-CD137scFv-IL-15 or RTX-DuoFab-CD137scFv-IL-15 and B cells (CD20+). The co-cultures were cultivated for 28 days. Every three to four days the NK cell number was determined and fresh media as well as antibody were added. Different B:NK cell ratios were analyzed. The x-fold expansion of the NK cells was plotted against the time in days. Each panel represents a different NK cell donor.

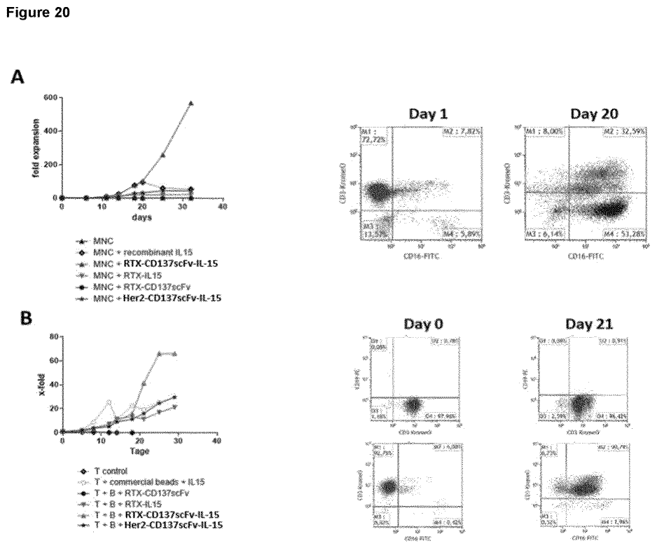

FIG. 20. Expansion of T cells. To analyze whether our novel fusion proteins are also capable in expanding T cells, MNC or CD3-positive T cells were isolated by MACS-sorting from healthy donors. To determine the impact of the different structural components and the impact of target cell presence, (A) freshly isolated MNC or (B) a mixture of purified B cells and T cells from healthy donors were co-incubated with fusion proteins (RTX-CD137scFv-IL-15; RTX-CD137scFv; RTX-IL-15; Her2-CD137scFv-IL-15). The cells were cultivated for up to 4 weeks. Every three to four days fresh media containing the respective fusion proteins were added. The fold expansion was plotted against the time in days. Data are presented as mean values of triplicate wells from one donor. The expanded cells were analyzed for T cell content by flow cytometry.

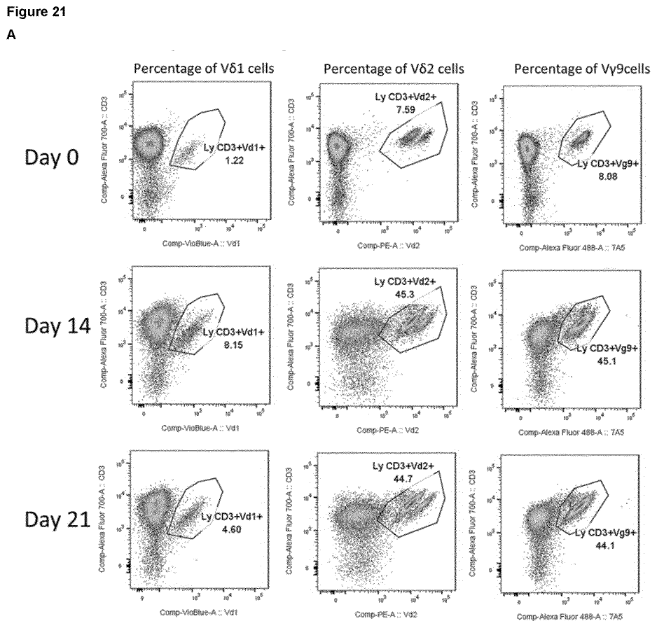

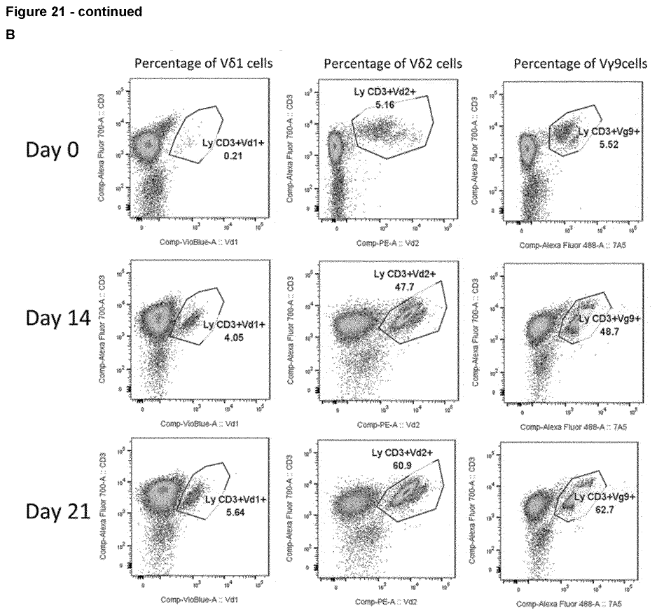

FIG. 21. RTX-CD137scFv-IL-15 triggers expansion of γδ T cells. B cells and T cells were isolated from PBMC of healthy donors and co-cultured in the presence of RTX-CD137scFv-IL-15. At day 0, 14 and 21 the content of vδ1 and vδ2 γδ T cells was analyzed by multi color flow cytometry and quantified. Together, these data demonstrate that RTX-CD137scFv-IL-15 was capable in triggering significant expansion of γδ T cells. A) Data from donor 1, B) Data from donor 2.

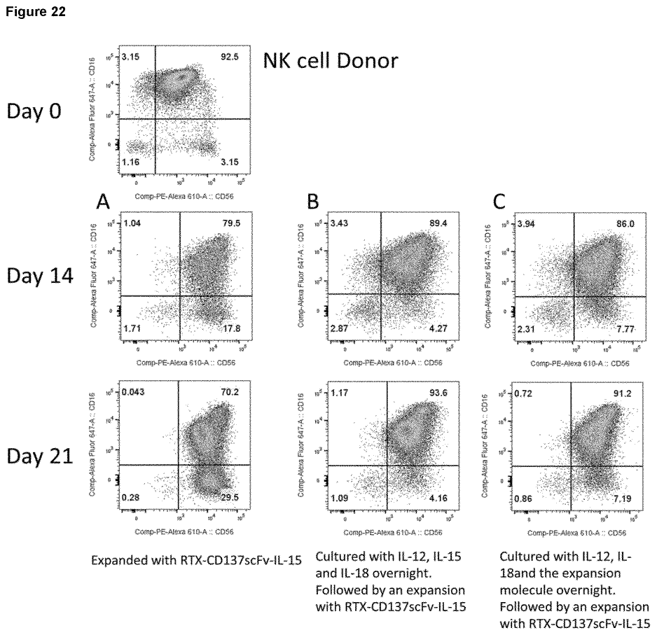

FIG. 22. Expansion of IL-12, IL-15, IL-18 stimulated NK cells to endow memory like properties. Culturing NK cells with IL-12, IL-15 and IL-18 overnight followed by an expansion with RTX-CD137scFv-IL-15 in the presence of B cells (B) as well as culturing the NK cells with IL-12, IL-18 and the expansion molecule RTX-CD137scFv-IL-15 overnight followed by an expansion phase with RTX-CD137scFv-IL-15 in the presence of B cells (C) showed higher percentages of CD56/CD16 double positive NK cells compared to NK cells that only underwent expansion with RTX-CD137scFv-IL-15 as single agent (A). Note: the expansion phase was carried out in the presence of B cells as described above. To assess the expression of FcγRIIIa (CD16a) flow cytometry analyses were performed with commercially purchased CD16, CD56 and antibodies at day 0, 14 and 21. Data show representative results.

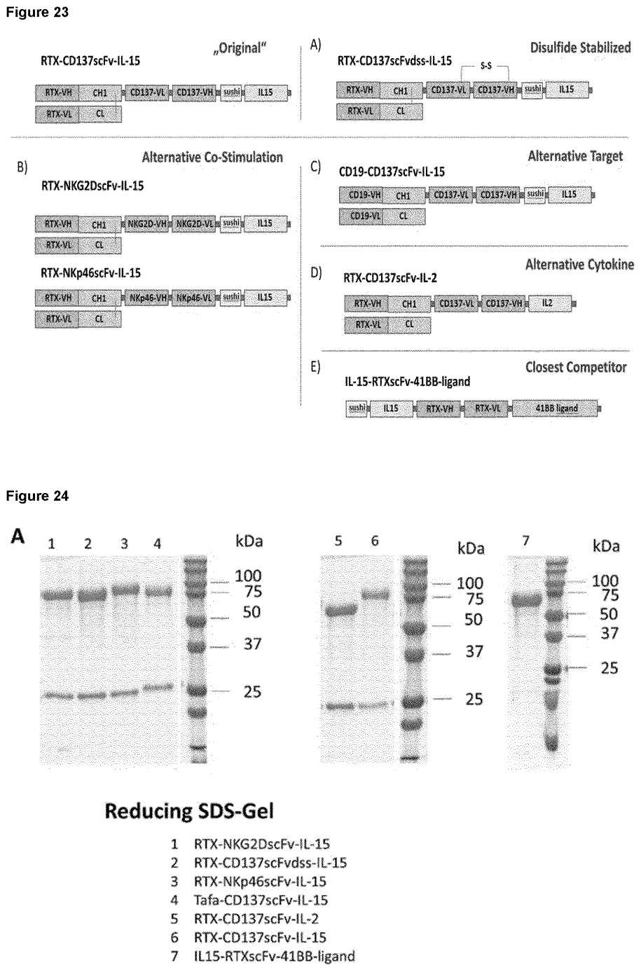

FIG. 23. Scheme of alternative molecule designs including closest competitor molecule design. Six additional molecule designs were realized to analyze the impact of different co-stimulatory receptors, alternative B cell targets, alternative cytokines, disulfide stabilization and closest competitor molecule based on the 4-1BB ligand to stimulate CD137 (Kerner, Mol Cancer Ther, 2014; Beha, Mol Cancer Ther, 2019).

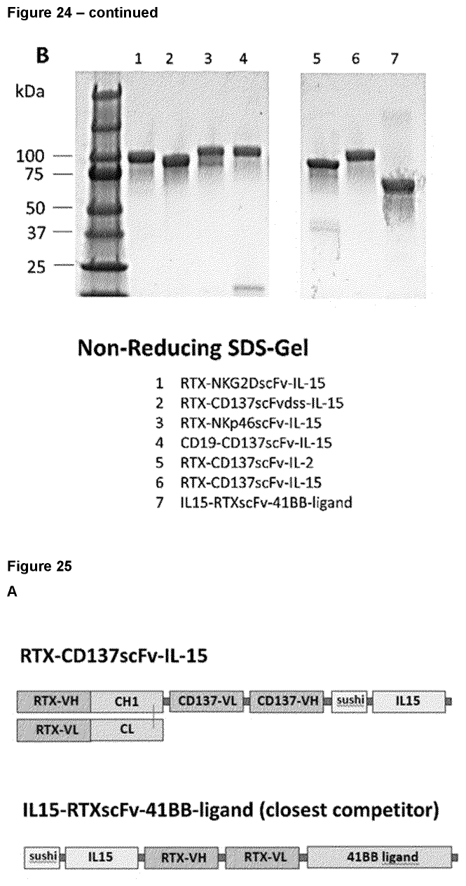

FIG. 24. The purified novel molecule variants show the expected molecule structure. The novel molecule variants derived from RTX-CD137scFv-IL-15 were produced by transient transfection in CHO—S cells and purified by affinity chromatography and size exclusion chromatography. In SDS-PAGE using reducing or non-reducing conditions the purified molecules show the expected molecule mass and assembly.

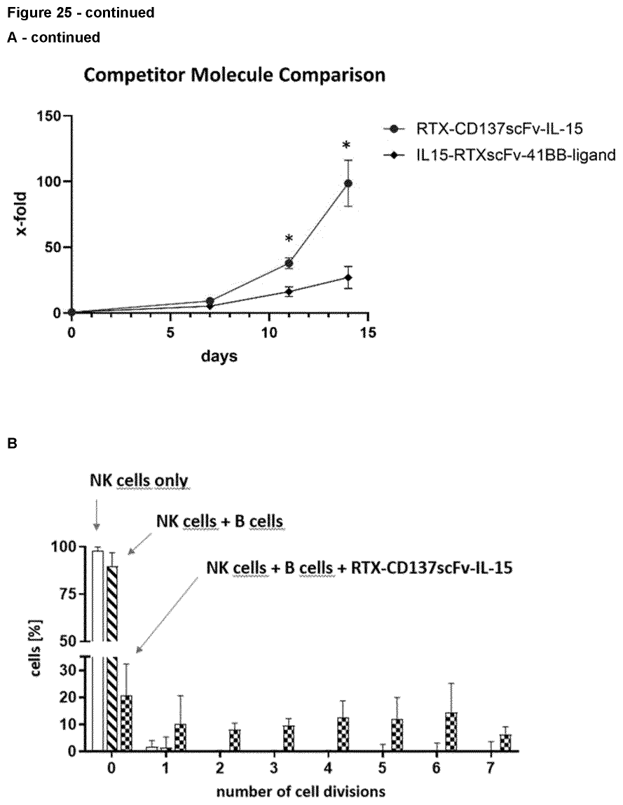

FIG. 25. Expansion capability of RTX-CD137scFv-IL-15 compared to IL15-RTXscFv-41BB-ligand. A) To compare the expansion capability of both molecules, freshly isolated NK cells from healthy donors were co-incubated with B cells and the respective molecules according to the expansion protocol described above. The comparison between RTX-CD137scFv-IL-15 (red, circle) and the IL15-RTXscFv-41BB-ligand competitor molecule unexpectedly revealed a significantly higher expansion capacity of RTX-CD137scFv-IL-15 than IL15-RTX-scFv-41BB-ligand although targeting the same surface structure on B cells and triggering the IL15 receptor and CD137 on NK cells. The x-fold expansion of NK cells was plotted against the time in days. Data are presented as mean values of three independent NK cell donors with error bars representing ±SEM; *=p<0.05. B) NK cells were labelled with CFSE and were either left untreated (NK cell only), were co-incubated with B cells (NK cells+B cells) or were co-incubated with B cells in the presence of RTX-CD137scFv-IL-15 (NK cells+B cells+RTX-CD137scFv-IL-15). After each cell division the CFSE signal is reduced/diluted, allowing precise measurement of cell divisions. After 5 days CFSE was measured by flow cytometry and cell divisions were calculated. Cells that underwent at least 1 cell division were considered proliferating. Data are presented as mean values of three independent NK cell donors with error bars representing ±SEM.

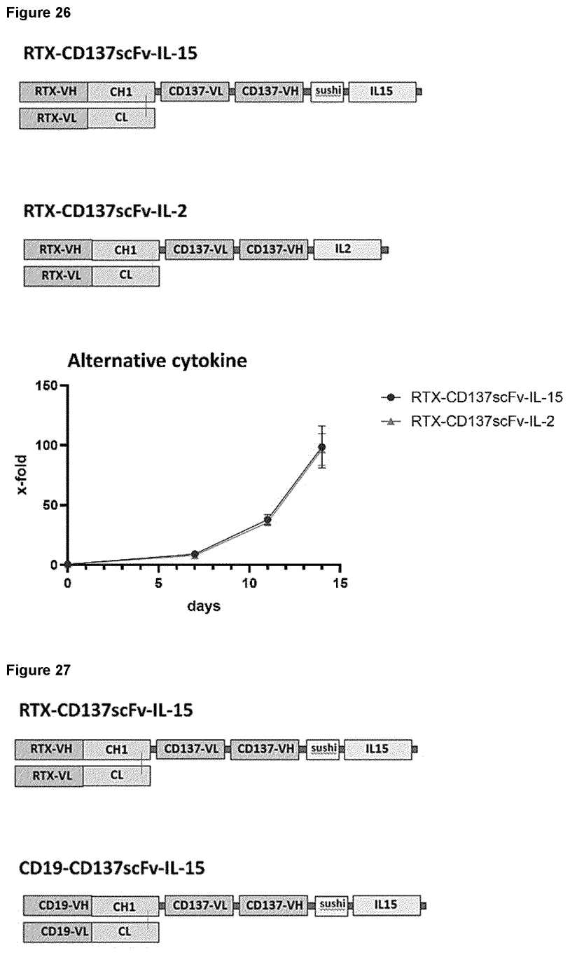

FIG. 26: Expansion capability of expansion molecule variants harboring IL-2 or Il-15. To compare the expansion capability of the expansion molecules freshly isolated NK cells from healthy donors were co-incubated with B cells and the respective molecules. Media was exchanged and protein was replenished as described above. Using IL-2 (RTX-CD137scFv-IL-2, triangle) instead of Il-15 (RTX-CD137scFv-IL-15, solid circle) in the design of our expansion molecule resulted in a similar capability to trigger the expansion of NK cells. The x-fold expansion of NK cells was plotted against the time in days. Data are presented as mean values of three independent NK cell donors with error bars representing±SEM.

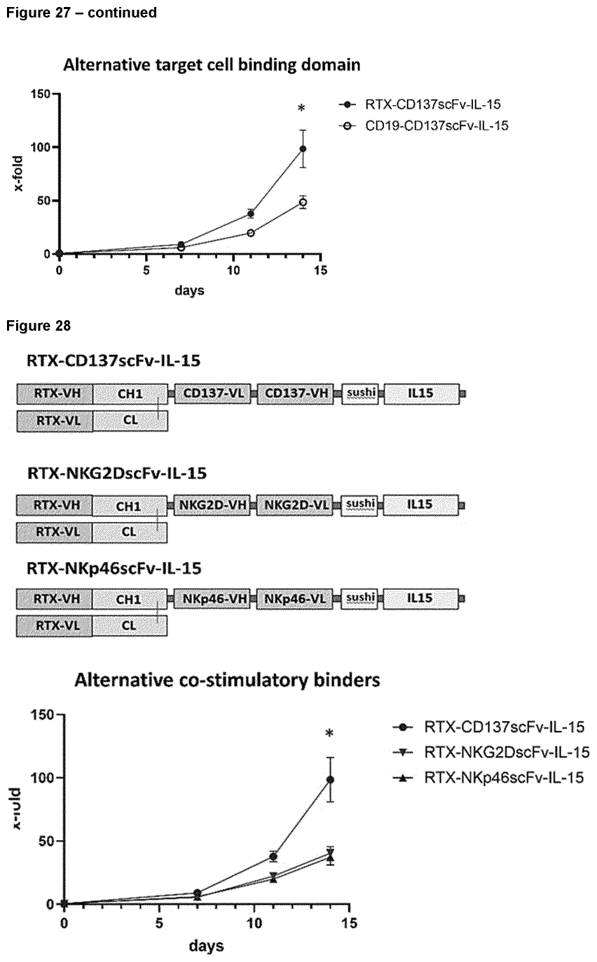

FIG. 27. Expansion capability of expansion molecule variants targeting CD19 or CD20. To compare the expansion capability of the expansion molecules freshly isolated NK cells from healthy donors were co-incubated with B cells and the respective molecules. Targeting CD19 (CD19-CD137scFv-IL-15, open circle) instead of CD20 (RTX-CD137scFv-IL-15, solid circle) on B cells resulted in a decreased capability to trigger the expansion of NK cells. The x-fold expansion of NK cells was plotted against the time in days. Data are presented as mean values of three independent NK cell donors with error bars representing±SEM. *=p<0.05

FIG. 28. Expansion capability of the expansion molecules with alternative “co-stimulatory” activity. To compare the expansion capability of the expansion molecules freshly isolated NK cells from healthy donors were co-incubated with B cells and the respective molecules. Utilizing antibody derivatives binding alternative co-stimulatory receptors on NK cells resulted in NK cell expansion, but to a lesser extent. Stimulation through CD137 (RTX-CD137scFv-IL-15, circle, ●), NKG2D (RTX-NKG2DscFv-IL-15, ▾), NKp46 (RTX-NKp46scFv-IL-15, ▴). The x-fold expansion of NK cells was plotted against the time in days. Data are presented as mean values of three independent NK cell donors with error bars representing±SEM.

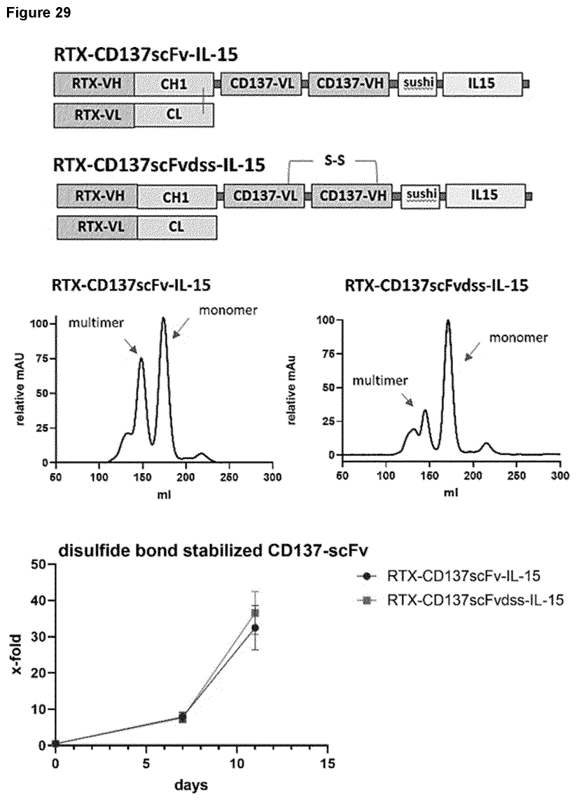

FIG. 29. Expansion capability of RTX-CD137scFv-IL15 compared to RTX-CD137scFvdss-IL15. To compare the expansion capability of both molecules freshly isolated NK cells from healthy donors were co-incubated with B cells and the respective molecule. The comparison between RTX-CD137scFv-IL-15 (circle) and the RTX-CD137scFvdss-IL-15, (square) showed no significant difference. Therefore, the inclusion of an additional disulfide bridge did not affect the expansion capacity of the molecule. However, a reduced multimer formation could be observed in size exclusion chromatography. The x-fold expansion of NK cells was plotted against the time in days. Data are presented as mean values of three independent NK cell donors with error bars representing ±SEM.

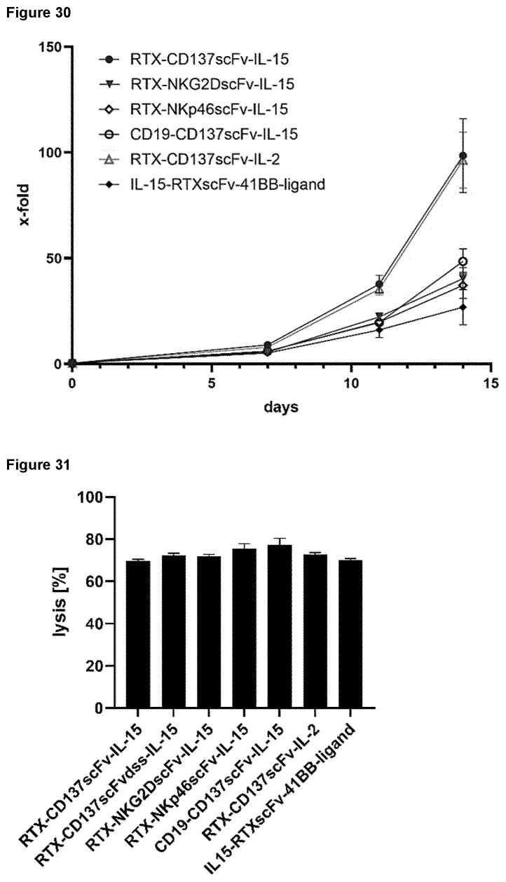

FIG. 30. Expansion capability of the expansion molecules-Summary. All molecules in the novel “target_Fab-scFv-cytokine” of the design as described herein show superior expansion rates compared to the closest competitor molecule design in the “cytokine-target_scFv-natural_ligand” design. Data are presented as mean values of three independent NK cell donors with error bars representing ±SEM.

FIG. 31. NK cells expanded with novel “alternative expander molecules” show potent natural cytotoxicity against sensitive K562 cells. Natural cytotoxicity of NK cells expanded with the novel fusion proteins were analyzed in classical 4 h Cr51 assays. K562 cells served as target cells. E:T ratio=10:1. Data are presented as mean values of three independent NK cell donors with error bars representing±SEM.

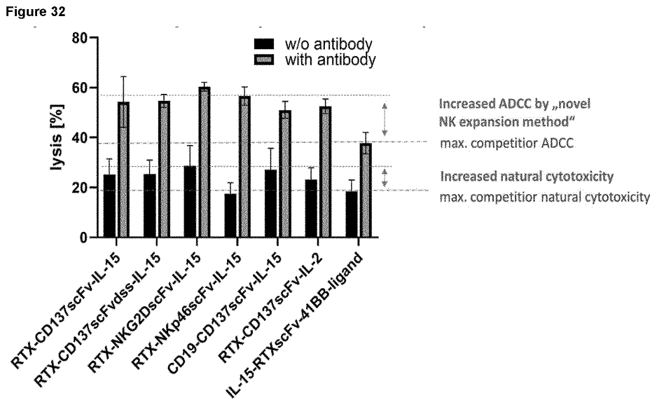

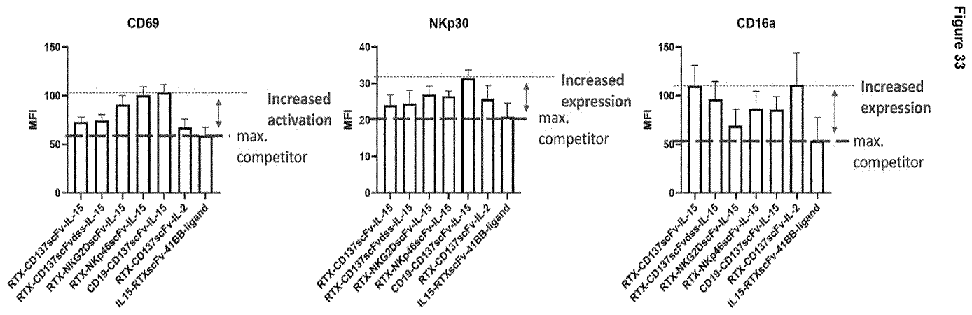

FIG. 32. NK cells expanded with novel “expander molecules” show increased natural cytotoxicity and improved ADCC activity. Natural cytotoxicity and ADCC activity of NK cells expanded with the novel fusion proteins were analyzed in classical 4 h Cr51 assays. GRANTA-519 cells which are less sensitive NK cell targets served as target cells. E:T ratio=10:1. To evaluate the ADCC capacity of expanded NK cells, rituximab (with antibody) was added (1 μg/ml final concentration). Data are presented as mean values of three NK cell donors with error bars representing±SEM.

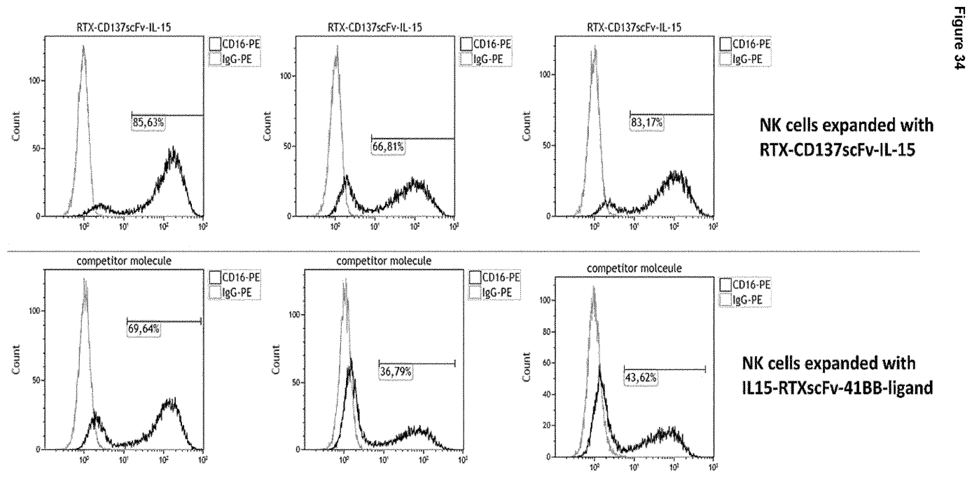

FIG. 33. NK cells expanded with novel “expander molecules” show an increased activation status compared to IL15-RTXscFv-41BB-ligand. NK cells expanded with the novel fusion proteins or competitor molecule were analyzed for the expression of the typical activation marker CD69, natural cytotoxicity receptor NKp30 and FcγRIIIa (CD16a) by flow cytometry. Mean values of three NK cell donors are displayed +/−SEM.

FIG. 34. NK cells expanded with RTX-CD137scFv-IL15 show a higher proportion of FcγRIIIa high expressing NK cells. NK cells expanded with RTX-CD137scFv-IL-15 or IL15-RTXscFv-41BB-ligand were analyzed for the expression of FcγRIIIa (CD16a) by flow cytometry. Results from three NK cell donors are displayed.

The Examples illustrate the invention.

### EXAMPLE 1

### Material & Methods

### Cell Culture

The cell lines were cultivated in suitable media at 6% atmospheric CO₂ and 37 °C. To keep the cells at an optimal density for continued growth the culture was divided two to three times per week. The passaging included the removal of the medium and the transfer of the cells from previous culture to fresh medium. Adherent cultures were washed with PBS and then detached with accutase or trypsin-EDTA.

Chinese hamster ovary CHO—S cells (FreeStyle™ CHO—ST Cells; R800-07, Thermo Fisher Scientific, Dreieich, GER) were cultivated in CD CHO medium (10743-011, Life Technologies, Carlsbad, USA) supplemented with 1% GlutaMax (Life Technologies) in a horizontal shaking incubator. After large scale electroporation-mediated transfection the CHO—S cells were cultured in CD OptiCHO™ (12681, Life Technologies) supplemented with 1% GlutaMax™ (35050-038, Life Technologies), 1% PLURONIC® F-68 (24040-032, Life Technologies), 1% HT-Supplement (11067-030, Life Technologies). 24 hrs. after the transfection 1 mM Na-butyrate (B5887, Sigma-Aldrich) was added to the cell culture and 48 hrs. after the transfection a daily feeding with 3.5% Feed Stock (CHO CD Efficient Feed™ A (A10234-01, Life Technologies incl. 14% Yeastolate Stock Solution (0.5% Difco™ TC Yeastolate, UF (292804, Life Technologies) soluble in H₂O), 3.5% GlutaMax™ (72400-021, Life Technologies) and 12.4% Glucose (Sigma-Aldrich)] was started. The fusion protein producing cells were cultivated in a 6% CO₂, 95% atmospheric moisture and 32 °C horizontal shaking incubator according to the manufacturer's recommendations.

Murine CTLL-2 (ATCC® TIB-214™, LGC Standards GmbH, Wesel, GER) were cultivated in RPMI-1640+GlutaMax™, 1 mM sodium pyruvate (Sigma-Aldrich), 10% inactivated fetal bovine serum (FBS; 1270-106, Life Technologies), 10% rat T-STIM™ with con A culture supplement (354115, Corning GmbH, Kaiserslautern, GER) 100 U/ml penicillin and 100 μg/ml streptomycin (15140-122, Life Technologies). Subculturing the cells before they reach a density around 2×10⁵ cells/ml, to an inoculation density 1-2×10⁴ viable cells/ml.

GRANTA-519 (mantle cell lymphoma; ACC 342, DSMZ, Braunschweig, GER) were cultured in DMEM medium (41965-039, Life Technologies) supplemented with 10% inactivated FBS, 100 U/ml penicillin and 100 μg/ml streptomycin. 2

L-363 (plasma cell leukemia; ACC 49, DSMZ, Braunschweig, GER) were cultured in RPMI-1640 medium (11835-030, Life Technologies) supplemented with 10% inactivated FBS, 100 U/ml penicillin and 100 μg/ml streptomycin.

B and NK cells were freshly prepared via human B Cell Isolation Kit II (130-091-151, Miltenyi Biotec, Bergisch Gladbach, GER) or human NK cell Isolation kit (130-092-657, Miltenyi Biotec). After the isolation B and NK cells were cultured in NK MACS Basal medium (Miltenyi Biotec) supplemented with 1% NK MACS Supplement (Miltenyi Biotec), 5% AB serum (inactivated, P30-2501, PAN-Biotech GmbH, Aidenbach, GER) and 0.78 nM IL-15 (Miltenyi Biotec) or 18.7 nM EFP for up to four weeks.

### Cloning and Production of Recombinant Fusion Proteins

To produce the fusion protein RTX-CD137scFv-IL-15; RTX-IL-15; RTX-CD137scFv; Her2-CD137scFv-IL-15 the individual de novo synthesized components (CD20 secretion leader; CD20-VH, CD20-VL, cDNA sequence coding for immunoglobulin heavy and light chain constant region from the CD20 antibody rituximab; CH1, CL, cDNA sequence coding for the human immunoglobulin heavy chain constant region 1 and the kappa light chain constant region; CD137 VL, CD137 VH, cDNA sequences coding for the variable heavy and light chain constant regions building a scFv with specificity for 4-1BB (CD137); sushi domain, hIL-15, cDNA sequence coding for sushi domain and human interleukin 15; GS15, GS20, cDNA sequence coding for a 15 amino acid flexible linker (G₄S)₃ and a 20 amino acid flexible linker (G₄S)₄; myc-tag, His-tag, cDNA sequence coding for the c-myc epitope and a hexahistidine tag; Eurofins, Ebersberg, Germany) of each fusion protein were cloned into the expression vector pSEC-tag2. Correctness of cloned sequences was confirmed by Sanger sequencing of final constructs. To produce the fusion proteins RTX-NKp46scFv-IL-15, RTX-NKG2DscFv-IL-15, RTX-CD137scFv-IL-2, RTX-CD137scFvdss-IL-15, CD19-CD137scFv-IL-15 and IL15-RTXscFv-41BB-ligand the full constructs were de novo synthesized and cloned in pCDNA3.1 (+) (Thermofischer Scientific, Geneart).

Two weeks before the transfection CHO—S cells were thawed. These cells were kept in culture at 3×10⁶ cells/ml, 6% CO2, 95% atmospheric moisture, 37 °C and 125 rpm in a shaking incubator. The day before the transfection, the cells were seeded at 2×10⁶ cells/ml. For the expression of the desired proteins, 10 times 8×10⁷ CHO—S cells (FreeStyle™ CHO—S™ Cells; R800-07 Thermo Fisher Scientific, Dreieich, GER) were transfected by performing electroporation using the MaxCyte Flow Electroporation® Unit STX, electroporation chamber OC-400 and the program “CHO—S protein expression” (MaxCyte Inc., Gaithersburg, USA) and endotoxin free produced vectors (120 μg/transfection-divided in 60 μg light chain vector and 60 μg heavy chain vector) according to the manufacturer's recommendations. Produced fusion proteins carrying a Fab-fragment were purified from cell culture supernatant with CaptureSelect™ IgG-CH1 Affinity Matrix (Thermo Fisher Scientific) and affinity chromatography using gravity flow columns (Bio-Rad Laboratories, Hercules, CA, USA) according to the manufacturer's recommendations. To purify the IL15-RTXscFv-41BB-ligand fusion protein the supernatant was dialyzed three times against wash buffer (50 mM NaH2PO4, 300 mM NaCl, 10 mM Imidazole, pH8) and then captured with Ni-NTA affinity chromatography according to the manufacturer's recommendations (Qiagen). Protein was eluted with elution buffer (50 mM NaH2PO4, 300 mM NaCl, 250 mM Imidazole, pH8). The protein was then dialyzed 3 times against PBS. To remove possible contaminations of aggregates size exclusion chromatography was performed by predefined methods using the ÄKTApure liquid chromatography system (Cytiva Europe GmbH, Freiburg™ Breisgau, GER). Protein concentrations were determined by using Pierce™ BCA Protein-Assay (Thermo Fisher Scientific) in accordance with the manufacturer's protocol.

### Sodium Dodecyl Sulfate-Polyacrylamide Gel Electrophoresis (SDS-PAGE) and Gel Filtration Chromatography

To determine the size and the purity of the proteins, Sodium Dodecyl Sulfate Polyacrylamide Gel Electrophoresis (SDS-PAGE) was performed. For the sample preparation, 5× loading buffer (either reducing or non-reducing) was added to 3 μg of each protein sample and the samples were heated up to 95 °C for 10 min to denature the proteins. After cooling down, the samples were applied on an SDS-bis-Tris-polyacrylamide gel (10%, (#456-1083, Bio-Rad Laboratories GmbH, Feldkirchen, GER). According to their molecular masses, the proteins were separated for 60 min at 120 V. Gels were dyed overnight at constant shaking in a Coomassie Brilliant Blue solution (Carl Roth GmbH, Karlsruhe, Germany).

### MTT Proliferation Assay

To test the functionality of the fused IL-15 component of the fusion proteins a colorimetric assay (MTT based; 11465007001, Cell Proliferation Kit I, Roche, Mannheim, GER) was used to quantify the metabolic cell activity. The MTT assay was performed using IL-2-dependent CTLL-2 cells. Due to the fact that IL-15 binds to the same receptor complex called IL-2R/IL-15RB and γC, CTLL-2 cells respond to IL-15 stimulation. After the starvation of the CTLL-2 cells (no addition of IL-2 (T-STIM) for 5 hours), 3×10⁴ cells were transferred into a 96-well plate and incubated at 37 °C and 6% CO₂ for 48 hrs. To test the functionality of the different proteins, these proteins as well as recombinant human IL-15 were added in an equimolar concentration range between 62.6 nM to 0.61 μM. The absorbance of solved formazan was measured spectrophotometrically at 570 nm with a Sunrise plate reader (Tecan Group Ltd., Mannedorf, CH). The evaluation was done with GraphPad PRISM 5.0.

### Flow Cytometry Analyses

All immunofluorescence analyses were performed on a Navios EX-Flow cytometer (Navios, Beckman Coulter, Brea, CA, USA). For each sample 1×10⁴ events were collected and dead cells and cellular debris were excluded by using appropriate forward and side scatter gates. Cells only stained with secondary antibody served as negative control. The measuring of the immunofluorescence occurred at the corresponding wavelength (FITC/PE). Final analyses were performed using Kaluza C Analysis Software as well as GraphPad Prism 4.0 Software.

### Target Cell Binding

To determine the Fab-mediated target cell binding of the different proteins, two different cell lines (GRANTA-519 for CD20; SK-BR-3 for 4D5) were used. Respectively 3×10⁵ cells were incubated with the different fusion proteins at a concentration of 4.85 UM in a serial dilution 1/5 on ice for 30 min, washed twice with 1000 μl PBA buffer (PBS including 1% BSA), and stained on ice for 30 min with FITC-conjugated anti-human kappa antibody (50 μg/ml, Southern Biotech, Birmingham, USA). After washing twice, cells were analyzed by performing flow cytometry analysis.

### 4-1BB Binding

In order to ascertain the 4-1BB-scFv binding of the fusion proteins, CEM-CRRF cells were stimulated to express CD137 on their cell surface. Stimulation occurred for 16 hrs. with 1.33 UM lonomycin (10634, Sigma-Aldrich) and 16.2 nM PMA (P1585, Sigma-Aldrich). Cells that were incubated under the same conditions but using DMSO instead of PMA and lonomycin served as negative control. Respectively 3×10⁵ cells were incubated with the different fusion proteins at a concentration of 4.85 UM in a serial dilution 1/5 on ice for 30 min. After washing twice with 1000 μl PBA buffer the cells were incubated 30 min on ice with FITC-conjugated anti-human kappa antibody (50 μg/ml, Southern Biotech, Birmingham, USA). Flow cytometry analyses were performed after washing the cells twice.

### Analysis of Cell Surface Markers

A total of 3×10⁵ cells were washed with PBA. Cells were then incubated with 5 μl directly labelled primary antibodies for 30 min on ice and in darkness. Cells which were incubated with corresponding IgG-Isotype control served as a negative control. After the incubation, the cells were washed once with 3 ml PBA and then resuspended in 500 μl PBA. The measuring of the immunofluorescence occurred at the corresponding wavelength (FITC/PE).

### Preparation of PBMC, Isolation of NK and B Cells and Verification

Citrate buffered blood samples from healthy donors or patients were obtained. To isolate peripheral blood mononuclear cells (PBMCs), Ficoll-Paque™ PLUS (Cytiva Europe) density centrifugation was performed for 20 min at 2500 rpm and room temperature without brake. The NK cell, T cells as well as the B cell isolation was performed with the corresponding isolation kits as described by the producer (human NK cell Isolation Kit, human B cell Isolation Kit II, pan T cell isolation kit; Miltenyi Biotec, Bergisch Gladbach, GER). Cell purity analyses were performed by flow cytometer analyses using suitable B, T and NK cell marker (CD56-APC [IM2474], CD3-ChromeOrange [B00068], CD16-FITC [B49215], CD19-PE [A86355], IgG1-APC [IM2475], IgG1-PE [A07796, A74764], IgG1-FITC [A07795], IgG1-ChromeOrange [A96415], Beckman Coulter GmbH, Krefeld, GER) with a cell sample taken before cell separation and a cell sample taken after isolation.

Fusion Protein Triggered NK Cell Expansion from Purified NK Cells or Patient Derived PBMCs

To determine the (tumor) cell-dependent expansion of NK cells, isolated NK cells were co-cultured with autologous target cells (e.g. B cells) in human NK MACS medium (+1% NK MACS Supplement, +5% AB serum) at the ratio 2:1 (starting condition 1.5×10⁶/ml). NK cells cultured without target cells as well as NK cells only served as a negative control. Purified NK cells expanded with the NK cell expansion and activation kit from Miltenyi (130-094-483, Miltenyi Biotec) according to the manufacturer's recommendations served as control. To determine the target cell dependent expansion of NK cells from isolated patient derived PBMC, the patient derived PBMCs, were incubated in human NK MACS medium (+1% NK MACS Supplement, +5% AB serum) at a starting condition of 1.0×10⁶/ml. PBMC cultured without EFP supplementation served as a negative control.

The co-cultures were cultivated in a 6% CO2, 95% atmospheric moisture and 37 °C incubator. Different fusion proteins (18.7 nM) were added to the co-cultured cells. 4 days after the starting point fresh medium, as well as fusion proteins were added. After day seven, new media as well as the fusion protein were added every three to four days (day 7, 11, 14, 18, 21, 25). On these days the total cell number was determined by trypan blue (T8154, Sigma-Aldrich) and Neubauer counting chamber (0640010, Paul Marienfeld GmbH & Co KG, Lauda-Königshofen, GER), and the cells were reseeded at a total of 1×10⁶ cells. On day 0 and day 7-29 cells were characterized by performing flow cytometry analysis with suitable NK cell marker (CD56-APC [IM2474], CD3-ChromeOrange [B00068], CD16-FITC [B49215], CD19-PE [A86355]; CD11a-FITC [IM0860U], CD44-PE [A32537], CD69-PE [IM1943], NKG2D [A08934], DNAM-1-PE, NKp30-PE [IM3709], NKp44-PE [IM3710], NKp46-PE [IM3711]; IgG1-APC [IM2475], IgG1-PE [A07796, A74764], IgG1-FITC [A07795], IgG1-ChromeOrange [A96415], Beckman Coulter GmbH, Krefeld, GER).

### Analysis of Cell-Mediated Cytotoxicity

To characterize the functionality of the expanded activated NK cells, 51chromium release assays in the presence and absence of therapeutic antibodies were performed. The capacity to trigger ADCC, using the expanded activated NK cells as effector cells, was performed by carrying out a standard ⁵¹Cr release. Expanded NK cells were co-cultured with the ⁵¹Cr incubated target cells at different effector to target cell (E:T) ratios (1:1, 1:2.5, 1:5, 1:10 and 1:20). CD20 expressing cells (either autologous B cells from healthy donors or CD20 expressing cell line GRANTA-519) or autologous CD138⁺ multiple myeloma cells from patients were used as target cells. Target cells were incubated with 51Cr for two hours. The monoclonal antibodies rituximab (RTX, 1 μg/ml; Roche, Basel, CH), elotuzumab (SLAMF7, 2 μg/ml; BMS, New York, USA) and daratumumab (CD38, 2 μg/ml; Janssen-Cilag GmbH, Neuss) in the autologous patient setting, were added. In all settings trastuzumab (4D5, 1 μg/ml; Roche, Basel, CH) served as a negative control. Co-cultures were incubated at 37 °C for four hours. Background lysis was detected by the expanded activated NK cells alone. Maximal lysis was detected by the addition of 1% Triton-X. The measurement was done by a MicroBeta Trilux 1450 LSC & Luminescence Counter (Perkin Elmer, Waltham, USA).

### Data Processing and Statistical Analyses

Graphical as well as statistical analyses were performed using GraphPad PRISM 4.0 (GraphPad Software Inc., San Diego, CA). The experiments were always performed under the same conditions. All data were analyzed and portrayed in the same way: all histograms were represented as mean±standard error of the mean (SEM) for at least three biological replicates and statistical statements were done by performing two-way analysis of variance (ANOVA) and Bonferroni post-tests. The null hypothesis was rejected for p<0.05.

### EXAMPLE 2

### Results for CD20 as Target on B-Cells

### Production and Purification

IL-15 and 4-1BB ligand trans-presentation on genetically modified target cells such as K562 or bead-based systems allow the expansion of NK cells ex vivo for cellular therapy. To obviate the requirement of genetically modified feeder cells or bead-based systems a novel multifunctional fusion protein was designed allowing specific NK cell and/or T cell expansion. To provide an Il-15 and 4-1BB signal to NK cells or T cells/NKT cells in trans, the fusion protein RTX-CD137scFv-IL-15 consisting of the Fab fragment of rituximab, an agonistic scFv directed against 4-1BB, the sushi domain of the IL-15 receptor and interleukin 15 (IL-15) was generated (FIG. 1, A). The protein is designed to bind CD20 on autologous B cells and thereby allows triggering the IL-15 receptor and 4-1BB on NK cells or T cells/NKT cells in trans. To investigate the contribution of the individual components of the molecule on NK cell activation and expansion, additional protein variants RTX-IL-15; RTX-CD137scFv; Her2-CD137scFv-IL-15 lacking certain building blocks of RTX-CD137scFv-IL-15 were generated.

For the production of proteins, CHO—S cells were co-transfected with the respective expression vectors that encoded the heavy chain derivatives and respective light chains. The purification of the fusion proteins from culture supernatants was performed by affinity chromatography. To remove possible residual contaminations and multimers or aggregates, quantitative size exclusion chromatography was performed. The purity and molecular mass of the isolated proteins was analyzed by SDS-PAGE and Coomassie Blue staining (FIG. 1, C). Under reducing conditions the light chain (LC) appeared at the calculated molecular mass of 25.7 kDa and the heavy chain derivatives (HC) at 52.6-79.3 kDa (RTX-CD137scFv-IL-15 (79.3 kDa); RTX-IL-15 (52.6 kDa); RTX-CD137scFv (55.7 kDa); Her2-CD137scFv-IL-15 (79.3 kDa)). Similar analyses using non-reducing conditions revealed the integrity of the respective proteins with molecular masses of: RTX-CD137scFv-IL-15 (105 kDa); RTX-IL-15 (78.3 kDa); RTX-CD137scFv (81.4 kDa); Her2-CD137scFv-IL-15 (105 kDa).

### Antigen Binding

To determine the binding ability of the fusion proteins, flow cytometry analyses were performed. Either Granta-519 cells (for CD20 binding; FIG. 1, D) or 4-1BB (CD137)-positive stimulated CRF-CEM cells (for the 4-1BB binding; FIG. 1, E) were used.

The binding capacity of the CD20-specific fusion proteins (RTX-CD137scFv-IL-15—red filled circle, EC₅₀ value of 231 nM; RTX-CD137scFv—blue filled square, EC₅₀ value of 205 nM; RTX-IL-15—lilac filled square, EC₅₀ value of 414 nM) showed no significant differences. Only at one concentration a significant difference compared to RTX-CD137scFv-IL-15 and RTX-CD137scFv could be observed for the RTX-IL-15 (FIG. 1, D). As expected, no binding on Her2-negative Granta-519 cells could be observed for trastuzumab (black filled triangle) and the Her2-specific control fusion protein (Her2-CD137scFv-IL-15—black circle).

The CD20-specific fusion proteins that contained the 4-1BB-specific scFv (RTX-CD137scFv-IL-15—red filled circle, EC₅₀ value of 139 nM; RTX-CD137scFv—blue filled square, EC₅₀ value of 81 nM) showed no significant differences in binding to CD137-positive CRRF-CEM cells. Significant differences between the CD20-specific fusion proteins that contained the 4-1BB-scFv (RTX-CD137scFv-IL-15—red filled circle; RTX-CD137scFv—blue filled square) and the Her2-specific fusion protein (Her2-CD137scFv-IL-15—black circle, EC₅₀ value of 511 nM) could be observed, indicating that the specific Fab-fragment used to design the fusion protein may impact the overall activity of the protein. No CD137 binding could be observed for fusion proteins lacking the 4-1BB-specific scFv (RTX-IL-15—lilac filled square) and trastuzumab (black filled triangle) (FIG. 1, E).

### Functional Activity of the IL-15 Component

To determine the functionality of the IL-15 component of the fusion proteins, a cell metabolic activity assay was performed by using the IL-15-responsive murine CTLL-2 cells (FIG. 1, F). To compare the stimulatory activity of the different fusion proteins, serial dilutions of the fusion proteins (RTX-CD137scFv-IL-15—red filled circle; RTX-CD137scFv—blue filled square; RTX-IL-15—lilac filled square; HER2-CD137scFv-IL-15—black circle) and recombinant human interleukin 15 (hIL-15, IL-15—black triangle) were prepared and applied in an equimolar amount. The hIL-15 served as a positive control. The relative metabolic activity (in %) was plotted against the protein concentration (in pM). The highest hIL-15 value was set to 100% and EC₅₀-values were calculated. Significant differences between the recombinant hIL-15 and the fusion proteins were observed. While the EC₅₀-value of the recombinant IL-15 was 0.014 nM, the fusion proteins containing IL-15 (RTX-CD137scFv-IL-15—red filled circle; RTX-IL-15—lilac filled square; HER2-CD137scFv-IL-15—black circle) showed EC₅₀-values between 9 nM (RTX-IL-15) and 14 nM (HER2-CD137scFv-IL-15—black circle; FIG. 1, F). The fusion proteins containing IL-15 (RTX-CD137scFv-IL-15—red filled circle; RTX-IL-15—lilac filled square; HER2CD137scFv-IL-15-black circle) showed no significant differences in activity. As expected, no stimulatory activity could be observed for the fusion protein lacking the IL-15 component (RTX-CD137scFv—blue filled square; FIG. 1, F).

### Expansion of NK Cells

To determine the capacity of the novel fusion protein carrying all structural components (RTX-CD137scFv-IL-15) to trigger NK cell expansion, freshly isolated NK cells were incubated with the fusion protein in the presence of autologous B cells. The fusion protein potently triggered NK cell expansion. Expansion rates between 10-10,000-fold were observed after 28 days (FIG. 2, A). While only one donor showed an expansion rate below 10-fold (8.33-fold) and one donor below 100-fold (52.05-fold), the majority of the donors showed an expansion rate between 100 and 10,000-fold (eleven Donors between 100 and 1,000-fold and six Donors between 1,000 and 10,000-fold). Compared to a commercial bead-based expansion system, the IL-15-based fusion protein showed slightly improved expansion rates although the differences did not reach statistical significance (FIG. 2, B). In a next set of experiments the requirement of the individual structural components and the necessity of providing IL-15/4-1BB signals by trans-presentation (by opsonizing B cells) to trigger NK cell expansion was analyzed (FIG. 2, C). The experiments demonstrated that especially the fusion protein carrying all structural components was able to trigger potent NK cell expansion. In the absence of B cells significantly lower expansion rates were observed, indicating that trans presentation was required to provide optimal IL-15/4-1BB signaling.

### Analysis of Activation

NK cell cytotoxic activity is controlled by a set of receptors that recognize the absence of self-proteins and presence of stress ligands on target cells. The NK cell activation is characterized by the increased expression of specific NK cell surface receptors. To determine the status of the NK cells that were expanded with the recombinant fusion proteins, flow cytometry-based analyses were performed. NK cells express different amounts of Fc receptor FcγRIIIa (CD16) depending on their activation status. To assess this expression flow cytometer analyses were performed with commercially purchased CD16, CD56 and CD3 antibodies at day 0 and 7, showing that the expansion with the novel fusion protein leads to a higher amount of CD56⁺ CD16⁺ NK Cells (76%) compared to the expansion with the bead-based commercial expansion system (54%; FIG. 3, A). The expression of other NK cell markers was determined at day 0 and day 28 of culture (FIG. 3, B). By comparing the receptor expression at the beginning of the expansion with NK cells that underwent expansion for 28 days, it could be shown that the expression of selected NK cell markers showed a significant increase (FIG. 3, B). Data represent mean values of three independent measurements with error bars representing±SEM, *=<0.05.

### Natural Cytotoxicity of Expanded NK Cells

To determine the cytolytic capacity of the expanded NK cells, NK cell-mediated tumor cell lysis was measured by performing cytotoxicity assays with different target cells. NK cells expanded by our novel fusion protein demonstrated potent lysis of K562 cells at various effector to target ratios (FIG. 4A). Significant lysis of >30% was already observed at a low E:T ratio of 1:1. In a next set of experiments lysis of a panel of tumor cells representative of different tumor entities were performed. Significant lysis was observed with all tumor cell lines tested (FIG. 4B). The extent of lysis ranged from 20% to 75%. To test whether the expanded NK cells are still physiologically regulated and therefore do not attack non-malignant target cells, cytotoxicity assays were performed by using autologous non-malignant B cells as target cells. No significant lysis was observed with non-malignant B cells as target cells, indicating that the highly activated expanded NK cells are still physiologically regulated (FIG. 4B). Experiments were performed at a fixed E:T ratio of 10:1. Data presented are mean values +/−SEM of three NK cell donors.

### ADCC Mediated by Expanded NK Cells

Besides natural cytotoxicity NK cells are capable in triggering antibody-dependent cell-mediated cytotoxicity (ADCC) by engagement of the FcγRIIIa. As demonstrated above, NK cells expanded by our novel fusion protein expressed high levels of FcγRIIIa on a large proportion of cells (>75%). To test NK cell-mediated ADCC, different tumor cell lines of the B cell lineage were used. Expanded NK cells demonstrated significant lysis of all tumor cell lines tested and lysis rates were enhanced by adding a tumor targeting monoclonal antibody (Rituximab, CD19-DE) (FIG. 5, A+B) at a fixed E:T ratio. In a next set of experiments NK cell-mediated ADCC with allogeneic NK cells at varying E:T ratios was analyzed (FIG. 5, C, left panel). Again, expanded NK cells significantly lysed target cells in the absence of a therapeutic antibody at varying E:T ratios. Tumor cell lysis was enhanced by adding the therapeutic antibody rituximab. Next, similar experiments were performed using autologous non-malignant B cells as target cells. Importantly, expanded NK cells were incapable in triggering lysis of non-malignant cells at high E:T ratios (FIG. 5, C, right panel) underlining that the highly activated expanded NK cells are still physiologically controlled and do not attack non-malignant cells. By adding the therapeutic antibody rituximab lysis of non-malignant B cells could be achieved. In a similar setting, NK cells expanded with our multifunctional fusion protein were compared to NK cells expanded by a bead-based commercial expansion system. While no significant differences in natural cytotoxicity and ADCC of non-malignant B cells was observed, our expanded NK cells more potently triggered ADCC against tumor cells (FIG. 5, D).

Expansion and Cytotoxic Activity of NK Cells from Multiple Myeloma Patients

In a next set of experiments, it was tested whether our novel fusion protein was capable in triggering expansion of NK cells from tumor patients. Mononuclear cells from peripheral blood or bone marrow aspirates of Multiple Myeloma patients were used. In these initial experiments expansion rates between 10-400-fold could be achieved (FIG. 6, A). No or only minor expansion was observed when using the control molecules further proving that all structural components in the molecule were necessary to trigger optimal NK cell expansion. Under these experimental conditions NK cells were preferentially expanded. On day 16 already 85% of the culture were NK cells that expressed high levels of FcγRIIIa (FIG. 6, B). Finally, primary tumor cells were tested in cytotoxicity assays. Primary myeloma cells were significantly lysed by allogeneic NK cells (FIG. 6, C, left panel). Killing of tumor cells was significantly enhanced by addition of the therapeutic antibody elotuzumab. In the autologous setting killing of tumor cells was only observed in the presence of therapeutic antibodies daratumumab or elotuzumab indicating that these tumor cells still provide strong signals to evade natural cytotoxicity.

### EXAMPLE 3

### Results for BCMA as Target on B-Cell Malignancies

To evaluate the broad applicability of the proposed concept, with BCMA a second target structure was evaluated. Since antibody valency is a critical parameter for the activity of many antibody derivatives, additional constructs with two Fab fragments (DuoFab) as targeting domains were designed.

Design and Expression of Fusion Proteins with Bivalent Target Antigen Binding Capacity

Four constructs (RTX-DuoFab-CD137scFv-IL-15, RTX-CD137scFv-IL-15, BCMA-DuoFab-CD137scFv-IL-15, BCMA-CD137scFv-IL-15, 7A) were produced in CHO—S cells by transient transfection and purified by affinity chromatography. Multimers and aggregates were removed by size exclusion chromatography (FIG. 7, B).

Biochemical Characterization of Fusion Proteins with Bivalent Target Antigen Binding Capacity

The purified DuoFab-based proteins were further analyzed by SDS-PAGE and Coomassie Blue staining or western blotting. The molecules showed the expected molecular mass of 150 kDa using non-reducing conditions with no signs of degradation. Using reducing conditions the molecules separate into the light chain and heavy chain derivative. The identity of the respective polypeptide chains was confirmed by western blot analysis using kappa light chain or poly-histidine specific antibodies (FIG. 8).

Binding Characteristics of Fusion Proteins with Bivalent Target Antigen Binding Capacity

To determine the binding ability of the antibody fragments, flow cytometry analyses were performed. Either CD20-positive Granta-519 cells (FIG. 9, A) or Lenti-X cells transfected with an expression vector coding for BCMA cDNA were used (FIG. 10, B). For CD20 binding analyses, RTX-DuoFab-CD137scFv-IL-15 and RTX-CD137scFv-IL-15 were compared, while BCMA-DuoFab-CD137scFv-IL-15 and BCMA-CD137scF-IL-15 served as negative controls. For BCMA binding analyses, BCMA-DuoFab-CD137scFv-IL-15 and BCMA-CD137scFv-IL-15 were compared, while RTX-DuoFab-CD137scFv-IL-15 and RTX-CD137scFv-IL-15 served as negative controls.

Dose-dependent binding was analyzed and EC₅₀-values as well as Kd-values were calculated. The highest determined relative mean fluorescence intensity (to value for cell surface retention) was set to 100% and all other values were normalized to this point. The adapted determined relative mean fluorescence intensity (rel. MFI in %) was plotted against the protein concentration (in nM). The dose response was plotted as a dose-response curve as well as a hyperbole curve for CD20 binding analysis (FIG. 9, B+C) and only as a hyperbole curve for BCMA binding analysis (10, C). In a cell surface retention assay dissociated molecules in the supernatant were removed at different timepoints. The remaining cell surface-bound molecules were determined by flow cytometry (FIGS. 9, D and 10, D).

Both the dose-response curve as well as the hyperbole curve analysis showed significant differences in the binding ability of the RTX-DuoFab-CD137scFv-IL-15 and the RTX-CD137scFv-IL-15 constructs. Compared to the RTX-CD137scFv-IL-15 (EC50: 740.7 nM), the RTX-DuoFab-CD137scFv-IL-15 (EC50: 278.4 nM) showed a 2.7-fold lower EC₅₀ value (FIG. 9, B). The hyperbole curve analysis confirmed this result allowing the calculation of respective Kd-values. The RTX-DuoFab-CD137scFv-IL-15 (Kd of 47.9 nM) showed a 2.9-fold lower Kd-value compared to the RTX-CD137scFv-IL-15 (Kd: 138.9 nM, FIG. 9, C). The cell surface retention assay reflected the previous findings. 30 min after the starting point only 50% of the rel. MFI was observed for the RTX-CD137scFv-IL-15. In contrast, the rel. MFI of the RTX-DuoFab-CD137scFv-IL-15 remained over 50% even after 180 min (FIG. 9, D).

Similar results were obtained in the second model system. The binding analysis plot showed significant differences in the binding ability of the BCMA-DuoFab-CD137scFv-IL-15 and the BCMA-CD137scFv-IL-15. The BCMA-DuoFab-CD137scFv-IL-15 (Kd: 56.5 nM) showed a 16.4-fold lower Kd-value compared to the BCMA-CD137scFv-IL-15 (Kd: 924.5 nM, FIG. 10 C). The preliminary cell surface retention assay confirmed the previous findings obtained with CD20-specific molecules (FIG. 10, D).

In summary, it could be shown that all produced antibody derivatives specifically bind to their respective target cells/target antigens. Moreover, both antibody derivatives that contain two Fab fragments show a significantly stronger binding ability compared to the monovalent antibody derivatives. This resulted in increased surface retention of the DuoFab-based molecules, which may especially be a favorable characteristic for in vivo application.

Antibody Derivatives with Monovalent and Bivalent Tumor Cell Binding Domains do not Differ in CD137 Binding

The antibody derivatives were designed to bind to CD137 on activated NK cells with the CD137-scFv fragment. After stimulation with PMA and ionomycin, CCRF-CEM cells express CD137 on the cell surface. Cells without a prior stimulation served as negative control. Before starting the binding analyses the antigen expression on the corresponding cells were confirmed (FIG. 11, A+B). To compare the different antibody derivatives, dose-dependent binding was analyzed, and EC₅₀-values were calculated. The highest determined relative mean fluorescence intensity was set to 100% and all other values were normalized to this point. The adapted determined relative mean fluorescence intensity (rel. MFI in %) was plotted against the protein concentration (in nM) (FIG. 11, C). The analysis confirmed that the antigen CD137 was only expressed on stimulated CCRF-CEM cells (FIG. 11, B). The non-stimulated CCRF-CEM cells showed no significant differences in the MFI compared to the isotype-control (FIG. 11, A). Stimulated CCRF-CEM cells were confirmed CD137-positive. The affinity of the different antibody variants showed no significant differences. All four antibody derivatives showed almost the same binding abilities against stimulated CCRF-CEM cells. It was shown that except BCMA-DuoFab-CD137scFv-IL-15 the antibody derivatives as expected do not bind to the CD137-non-stimulated CCRF-CEM cells. In summary, it could be demonstrated that all analyzed antibody derivatives bound to the antigen CD137 and therefore contained a functional active CD137-scFv.

The IL-15-Dependent Stimulatory Activity does not Differ Between the Different Antibody Derivatives.

To determine the IL-15 functionality of the antibody derivatives, a cell metabolic activity assay was performed by using CTLL-2 cells. CTLL-2 is a murine cell line that is IL-2 dependent and responds to IL-15 stimulation. To compare the stimulatory activity of the different antibody derivatives, serial dilutions of the antibody derivatives and recombinant human interleukin 15 (hIL-15) were prepared and the dilutions were applied at equimolar concentration. The hIL-15 served as a positive control. The relative metabolic activity (in %) was plotted against the protein concentration (in pM) (FIG. 12). The highest hIL-15 value was set to 100% and EC₅₀-values were calculated.

The IL-15 dependent cell activity showed significant differences between the recombinant IL-15 and the IL-15 based antibody derivatives. While the EC₅₀-value of the recombinant IL-15 was 2.28 μM, the antibody derivatives showed EC₅₀-values between 11,640 PM and 17,942 μM (FIG. 12). Therefore, the recombinant IL-15 showed a 6,612 times higher activity compared to the antibody derivatives. As shown in FIG. 12 the set of antibody derivatives showed no significant differences in terms of CTLL-2 stimulation.

MonoFab and DuoFab Derivatives do not Differ in their Capacity to Trigger NK Cell Expansion.

In a next set of experiments the different molecules with monovalent or bivalent target antigen binding capacity were compared in terms of NK cell expansion (FIG. 13). No significant difference in their capacity to trigger NK cell expansion was observed, indicating that bivalent target antigen binding has no significant impact on this characteristic ex vivo (FIG. 13).

NK Cells that were Expanded with the Antibody Derivatives Showed an Activated Phenotype

NK cell activation is controlled by a set of receptors that recognize the absence of self-proteins and presence of stress ligands on target cells. The NK cell activation is characterized by the increased expression of specific NK cell markers. Typically, these markers include the antigen CD69 and the hyaluronate receptor CD44 as well as NKp44. Other activating receptors are NKp30, NKp46, CD16a, DNAM-1 and NKG2D. In the setting of tumors and chronic infections, NK cells can show an exhausted phenotype (Gardiner, 2017). The altered phenotypes are characterized by the downregulation of certain receptors like NKG2D, CD16a, NKp30, NKp44, and NKp46. Altered phenotypes are associated with decreased effector functions and therefore with poor control of malignancies or infections. To determine the status of the NK cells that were expanded with the antibody derivatives, flow cytometry-based analyses were performed. The expression of different NK cell markers was determined at the beginning and at the end of the NK cell expansion phase (FIG. 14). The NK cell marker expression was set synonymous with the fluorescence intensity. By comparing the receptor expression at the beginning of the expansion with NK cells that underwent expansion for 28 days, it was shown that the expression of specific NK cell markers showed a significant increase (FIG. 14).

For NK cells expanded with the RTX-CD137scFv-IL-15 construct, the NK cell markers CD44, NKp44, CD69 and DNAM-1 showed a significantly increased expression level. On day zero, the expression of the hyaluronate receptor CD44 showed a fluorescence intensity of 167.9 MFI. At the end of the expansion the expression increased to 645.7 MFI. The expression of NKp44 was 12-fold increased. Similarly, the expression of CD69 and of the DNAX Accessory Molecule-1 (DNAM-1) was significantly increased. All other receptors analyzed showed also increased surface expression but the differences did not reach statistical significance. Similar results were obtained for NK cells expanded with the RTX-DuoFab-CD137scFv-IL-15 molecule.

The determination of the NK cell marker expression of the NK cells that were incubated with the BCMA antibody derivatives showed a different expression pattern. Among all tested samples only NKp44 and CD69 showed significant differences. No differences between the DuoFab and MonoFab-based constructs were observed.

NK Cells Expanded with DuoFab-Based Fusion Protein are not Cytotoxic Against Non-Malignant B Cells but Mediate ADCC.

Since the expanded NK cells show an activated phenotype, it is important to analyze whether these cells show cytotoxic activity against non-malignant cells. Non-malignant autologous B cells were used as target cells in chromium release assays. Even at a high E:T ratio of 20:1 no lysis of non-malignant B cells was observed (FIG. 15). When the non-malignant B cells were opsonized with the CD20-specific antibody rituximab, significant lysis was triggered (FIG. 15). These data demonstrate that similar to our results with monovalent targeting fusion proteins NK cells expanded with bivalently binding fusion proteins are still physiologically regulated and are able to discriminate between non-malignant and malignant tissue. These inhibitory self-recognition signals can be overcome by strong activating signals, such as FcγRIIIa-triggering by antibody Fc domains.

NK Cells Expanded with DuoFab-Based Fusion Protein are Cytotoxic Against Lymphoma Cells and Cytotoxic Activity could be Enhanced by Combination with a Monoclonal Antibody.

In contrast to the results obtained with non-malignant B cells as target cells, lymphoma cells (GRANTA-519 cells) were significantly lysed by expanded NK cells (FIG. 16 and FIG. 17). No significant differences were observed between NK cells expanded by monovalent or bivalent fusion proteins. The cytotoxic capacity of the NK cells was further enhanced when a therapeutic antibody, such as rituximab was added (FIG. 16 and FIG. 17).

Impact of Frequency of NK Cell Stimulation with B Cells/Target Cells and NK Cell-to-Target Cell Ratio on NK Cell Expansion.

In order to further optimize the expansion procedure two additional parameters were analyzed which may impact the magnitude of NK cell expansion. In a first set of experiments the frequency of NK cell stimulation with target cells was analyzed (FIG. 18). While for the monovalently targeting molecule two stimulations seemed optimal, bivalently targeting molecules were maximally expanded with four stimulations in 2 out of 3 donors. For both types of molecules a 4:1 NK:target cell ratio was optimal to achieve maximum NK cell expansion (FIG. 19).

### Summary

With the BCMA fusion proteins it was demonstrated that the concept for ex vivo/in vivo targeted expansion of NK cells using the fusion proteins of the invention is broadly applicable to various target structures on NK target cells. Alternative molecule designs to bind target cells bivalently were successfully realized by the fusion of a second Fab fragment. No significant differences in terms of NK cell expansion and cytotoxic activity of the expanded NK cells were observed between monovalent or bivalent targeting molecules. The main functional difference of these molecules was observed in the surface retention assay. Bivalently targeting molecules showed prolonged surface retention a characteristic that is advantageous for in vivo application.

### EXAMPLE 4

### Expansion of T Cells

The IL-15 receptor and CD137 is also expressed by different subsets of (activated) T cells, Therefore, our novel fusion proteins may also be suitable for ex vivo and in vivo expansion of T cells. In a first set of experiments isolated MNCs (FIG. 20, A) or a coculture of purified T cells and B cells (FIG. 20, B) were incubated in the presence of the different fusion proteins, either containing all structural components that were necessary to expand NK cells or derivatives lacking one of the key components. Similar to the results observed with NK cells, also T cells were significantly expanded, and this effect was most pronounced with the construct containing all structural components. Interestingly, also CD3+/CD16+ T cells were significantly expanded. These cells are most likely NKT and/or γδ T cells. Together these data clearly demonstrate that our novel fusion protein not only potently triggers expansion of NK cells but also of CD3-positive T cell populations.

In subsequent experiments purified T cells (pan T cell isolation kit, Miltenyi Biotec) and isolated B cells from the same donor were co-cultured in the presence of RTX-CD137scFv-IL-15 according to the protocol used to expand NK cells. On day 0, 14 and 21 the content of vδ1 and vδ2 γδ T cells was analyzed by multi-color flow cytometry (FIG. 21A/B). A significant expansion of both vδ1 and vδ2 γδ T cells was observed for both donors analyzed. Up to 1239-fold expansion for vδ1 T cells and 546-fold expansion for vδ2 γδ T cells was measured. Together, these data demonstrate that RTX-CD137scFv-IL-15 was capable in triggering significant expansion of γδ T cells which also represent attractive immune effector cells to develop immunotherapies.

### EXAMPLE 5

### Expansion of Cytokine-Induced Memory-Like NK Cells

Memory-Like NK cells due to their increased cytolytic capacity represent an interesting immune effector cell population for therapeutic application. Overnight culture of NK cells in IL-12, IL-15 and IL-18 results in polarization of NK cells towards a memory-like phenotype (Romee, Blood, 2012; Romee, Sci Transl Med, 2016). Here, it was tested whether NK cells polarized towards a memory-like phenotype could be expanded by our novel fusion protein RTX-CD137scFv-IL-15. Three different assay conditions were compared: expansion with RTX-CD137scFv-IL-15 as single agent in the presence of B cells as described above (FIG. 22A), culturing NK cells with IL-12, IL-15 and IL-18 overnight followed by an expansion phase with RTX-CD137scFv-IL-15 in the presence of B cells (FIG. 22 B) as well as culturing the NK cells with IL-12, IL-18 and the expansion molecule RTX-CD137scFv-IL-15 overnight followed by an expansion phase with RTX-CD137scFv-IL-15 in the presence of B cells (FIG. 22 C). Before, during and after the expansion phase the NK cells were assessed for the expression of FcγRIIIa (CD16a) by flow cytometry using commercial CD16 and CD56 antibodies (day 0, 14 and 21). Data show representative results. Assay conditions B and C delivered higher percentages of CD56/CD16 double positive cells (>90%) compared to NK cells that only underwent expansion with RTX-CD137scFv-IL-15 as single agent (>70%).

### EXAMPLE 6

### Design of Alternative Molecule Variants and Comparison with a Fusion Protein Based on the Natural 4-1BB Ligand (Closest Competitor Molecule Design)

To further explore the potential of the prototype molecule designs and to get more insight into the relative contribution of individual molecule components to expand NK cells, alternative molecule designs were evaluated (FIG. 23). A) a disulfide stabilized version of RTX-CD137scFv-IL-15 was designed with the aim to reduce multimer formation of the protein during production. B) Protein variants harboring scFv fragments binding to alternative co-stimulatory receptors (NKG2D, NKp46) were designed to evaluate the impact of altered co-stimulation via alternative activating receptors on NK cells. C) A molecule addressing an alternative target structure (CD19) on B cells was designed to further analyze the impact of the target structure on B cells on NK cell expansion. D) IL-15 was replaced by IL-2 to evaluate the impact of using alternative cytokines in the molecule design. E) The closest competitor molecule design (IL15-RTXscFv-41BB-ligand) using the natural ligand (4-1BB ligand) to stimulate CD137 and a B cell targeting scFv derived from the Fab fragment (rituximab) used in our design was evaluated for its capacity to expand NK cells. The molecule architecture and linker sequences in IL15-RTXscFv-41BB-ligand were designed according to Kermer et al. (Mol Cancer Ther, 2014).

All molecules were expressed in CHO—S cells by transient transfection as described above. Fab-containing molecule formats were purified by CH1-specific affinity chromatography while the IL15-RTXscFv-41BB-ligand molecule was purified by Ni-NTA chromatography using the incorporated His-Tag using standard procedures. After extensive dialysis against PBS, all proteins underwent preparative size exclusion chromatography to remove aggregates. After size exclusion chromatography the proteins were quantified by the BCA method and analyzed by SDS-PAGE analysis and Coomassie staining using reducing or non-reducing conditions. All proteins showed the expected migration characteristics according to calculated molecular masses of the proteins. Due to their molecular design Fab-based fusion proteins demonstrate two protein bands using reducing conditions representing the light chain and the respective heavy chain derivative (FIG. 24A). No signs of degradation or contaminants were visible, confirming correct production of the molecules. Evaluation using non-reducing conditions demonstrated correct assembly of Fab-based two-chain molecules (FIG. 24 B, lanes 1-7). The IL15-RTXscFv-41BB-ligand molecule consists of non-covalently linked trimers (via the assembly of the 4-1BB ligand), therefore migration at a molecular mass of the monomer is expected in SDS-PAGE (FIG. 24 B, lane 7). Correct assembly of this trimeric protein was confirmed by size exclusion chromatography (data not shown).

In a first set of experiments RTX-CD137scFv-IL-15 and IL15-RTXscFv-41BB-ligand was compared for their capacity to expand purified NK cells with assay settings described above. After 14 days a 98.5-fold expansion was measured for RTX-CD137scFv-IL-15, while IL15-RTXscFv-41BB-ligand showed a 26.4-fold expansion rate (FIG. 25). These data demonstrate that the molecular design of RTX-CD137scFv-IL-15 and probably using an agonistic CD137-scFv instead of the natural 4-1BB ligand resulted in significantly improved expansion rates although triggering identical receptors on B cells and NK cells. To investigate the kinetics of proliferation, NK cells were labelled with CFSE (Carboxyfluorescein succinimidyl ester; CellTrace™ CFSE Cell Proliferation Kit) and were either left untreated (NK cell only), were co-incubated with B cells (NK cells+B cells) or were co-incubated with B cells in the presence of RTX-CD137scFv-IL-15 (NK cells+B cells+RTX-CD137scFv-IL-15). After each cell division the CFSE signal is reduced/diluted, allowing precise measurement of cell divisions. After 5 days CFSE was measured by flow cytometry and cell divisions were calculated. Cells that underwent at least 1 cell division in 5 days were considered proliferating. Interestingly, on day 5 about 80% of NK cells were proliferating (FIG. 25 B). This feature to drive a significant proportion of NK cells into proliferation may explain the superior capacity in NK cell expansion compared to IL15-RTXscFv-41BB-ligand. The findings are in line with published data of the NK cell activation capacity of a Fap-directed fusion protein with identical design as IL15-RTXscFv-41BB-ligand, which showed no significant capacity in triggering NK cell proliferation on day 4 after stimulation (Beha, Mol Cancer Ther, 2019). Interestingly, even an alternative improved molecule design using a single chain 4-1BB trimer (format: IL15-scFv-sc4-1BB) only triggered proliferation in about 15% of NK cells at high protein concentrations (Beha, Mol Cancer Ther, 2019). This maybe unexpected differential behavior of our novel fusion proteins compared to the published fusion proteins may suggest that an alternative molecule design and using an agonistic CD137-scFv instead of the natural 4-1BB ligand provides a qualitative different co-stimulatory signal, resulting in a significantly improved capacity to trigger NK cell expansion.

When in the molecule design IL-15 was replaced by IL-2 the resulting fusion protein RTX-CD137scFv-IL-2 demonstrated almost identical expansion rates compared to RTX-CD137scFv-IL-15 (FIG. 26). These data indicate that in terms of expansion IL-2 could replace IL-15 in our molecule design. Due to the intrinsic capacity of IL-2 to efficiently activate Tregs, this molecule could potentially be interesting to expand regulatory T cells (Harris, Clin Exp Immunol, 2023).

In the fusion protein CD19-CD137scFv-IL-15 the v regions in the CD20-Fab fragment were replaced by v regions with CD19 specificity. Interestingly, CD19-CD137scFv-IL-15 showed significantly reduced expansion capacities compared to RTX-CD137scFv-IL-15, indicating that the biology, epitope specificity and/or affinity of the targeting device and surface expression level of the addressed target antigen on B cells plays a critical role in the extend of NK cell proliferation (FIG. 27).

By replacing the CD137-scFv by a NKG2D-scFv or a NKp46-scFv, the impact of the co-stimulatory signal provided by our expansion molecules was evaluated in more detail. All molecules triggered significant proliferation of NK cells. Interestingly, CD137 co-stimulation resulted in superior expansion rates, indicating that the quality of the co-stimulatory signal has a significant impact on the capacity of the molecules to trigger NK cell expansion.

Finally, a disulfide stabilized molecule variant in which cysteines were introduced in the CD137-scFv at consensus framework regions (according to Brinkmann, PNAS, 1993) was generated and analyzed for its tendency to form multimers/aggregates and NK cell proliferation capacity. The disulfide bond stabilized molecule RTX-CD137scFvdss-IL-15 showed reduced levels of multimers after the first chromatography step, while retaining full NK cell expansion capacity compared to RTX-CD137scFv-IL-15. Therefore, this novel molecule design showed improved production properties.

In summary, all novel molecule designs tested under the described assay conditions showed improved NK cell expansion properties compared to IL15-RTXscFv-4-1BB-ligand (FIG. 30).

In a next set of experiments the cytolytic capacity of NK cells expanded with the various fusion proteins were analyzed in classical chromium release assays. K562 cells which are very sensitive to NK cell-mediated lysis were potently killed by all expanded NK cell preparations (FIG. 31). Interestingly, when GRANTA-519 lymphoma cells were used as target cells which are less sensitive to NK cell-mediated killing, RTX-NKp46scFv-IL-15 expanded NK cells were as effective as NK cells expanded with the IL15-RTXscFv-41BB-ligand competitor molecule to lyse target cells. Importantly, all other molecules showed increased natural cytotoxicity superior to IL15-RTXscFv-41BB-ligand (FIG. 32). Most strikingly, all NK cell preparations expanded with either of the novel expansion molecules were significantly more effective in mediating ADCC (in the presence of rituximab, with antibody) compared to NK cells expanded with IL15-RTXscFv-41BB-ligand competitor molecule (FIG. 32, with antibody). In summary, these data demonstrate that our novel fusion proteins compared to IL15-RTXscFv-41BB-ligand were more efficient in triggering expansion of NK cells and the expanded NK cells were more effective in mediating natural cytotoxicity and ADCC in combination with monoclonal antibodies (rituximab).

To investigate the underlying mechanism resulting in the difference in cytotoxic activity of the novel fusion proteins and IL15-RTXscFv-41BB-ligand competitor molecule, the expression of selected surface markers was investigated. Interestingly, increased expression of CD69, NKp30 and FcγRIIIa was observed (FIG. 33). While increased levels of NKp30 may explain the higher levels of natural cytotoxicity, clearly a higher expression of FcγRIIIa explains the superior ADCC activity. Further analysis revealed that a higher proportion of FcγRIIIa high expressing NK cells is found in NK cell expansions with the novel fusion protein RTX-CD137scFv-IL-15 compared to NK cells expanded with IL15-RTXscFv-41BB-ligand competitor molecule (FIG. 34).

Together, these data show a clear distinction in terms of extent of expansion and cytolytic activity between the novel fusion proteins as provided herein and previous state of the art IL15-RTXscFv-41BB-ligand competitor molecule design based on the natural 4-1BB ligand.

### REFERENCES

Albanell, J., & Baselga, J. (1999). Trastuzumab, a humanized anti-HER2 monoclonal antibody, for the treatment of breast cancer. Drugs Today, 35(12), 931-946.
Andrade, L. F. De, Huntington, N. D., Smyth, M. J., Martinet, L., Andrade, L. F. De, Guillerey, C., . . . . Liu, J. (2015). DNAM-1 Expression Marks an Alternative Program of NK Cell Maturation. CellReports, 11(1), 85-97. https://doi.org/10.1016/j.celrep.2015.03.006
Barillé-nion, S., Barlogie, B., Bataille, R., Bergsagel, P. L., Epstein, J., Fenton, R. G., . . . . Tricot, G. (n.d.). Advances in Biology and Therapy of Multiple Myeloma.
Becker, N. (2011). Epidemiology of Multiple Myeloma. Genetics (Vol. 183).
Becker, P. S. A., Suck, G., Nowakowska, P., Ullrich, E., Seifried, E., Bader, P., . . . . Seidl, C. (2016). Selection and expansion of natural killer cells for NK cell-based immunotherapy. Cancer Immunology, Immunotherapy, 65(4), 477-484. https://doi.org/10.1007/s00262-016-1792-y
Beha N, Harder M, Ring S, Kontermann R E, Müller D. IL15-Based Trifunctional Antibody-Fusion Proteins with Costimulatory TNF-Superfamily Ligands in the Single-Chain Format for Cancer Immunotherapy. Mol Cancer Ther. 2019 July; 18(7):1278-1288. doi: 10.1158/1535-7163.MCT-18-1204. Epub 2019 Apr. 30.
Bello, C., & Sotomayor, E. M. (2007). Monoclonal Antibodies for B-Cell Lymphomas: Rituximab and Beyond. Hematology, (June), 7-10.
Borrego, F., Robertson, M. J., Ritz, J., & Solana, R. (1999). CD69 is a stimulatory receptor for natural killer cell and its cytotoxic effect is blocked by CD94 inhibitory receptor, 12, 159-165.
Bottino, C., Castriconi, R., Pende, D., Rivera, P., Nanni, M., Carnemolla, B., . . . . Moretta, A. (2003). Identification of PVR (CD155) and Nectin-2 (CD112) as Cell Surface Ligands for the Human DNAM-1 (CD226) Activating Molecule, 198(4). https://doi.org/10.1084/jem.20030788
Boye, J., Elter, T., & Engert, A. (2003). An overview of the current clinical use of the anti-CD20 monoclonal antibody rituximab, (FIG. 1), 520-535. https://doi.org/10.1093/annonc/mdg175
Brandt, C. S., Baratin, M., Yi, E. C., Kennedy, J., Gao, Z., Fox, B., . . . . Levin, S. D. (2009). The B7 family member B7-H6 is a tumor cell ligand for the activating natural killer cell receptor NKp30 in humans, 206(7), 1495-1503. https://doi.org/10.1084/jem.20090681
Brinkmann U, Reiter Y, Jung S H, Lee B, Pastan I. A recombinant immunotoxin containing a disulfide-stabilized Fv fragment. Proc Natl Acad Sci USA. 1993 Aug. 15; 90(16):7538-42. doi: 10.1073/pnas.90.16.7538.
Britton, J. A., Khan, A. E., Rohrmann, S., Becker, N., Linseisen, J., Nieters, A., . . . . Ferrari, P. (2008). Anthropometric characteristics and non-Hodgkin's lymphoma and multiple myeloma risk in the European Prospective Investigation into Cancer and Nutrition (EPIC), 93(11). https://doi.org/10.3324/haematol. 13078
Bruenke, J., Fischer, B., Barbin, K., Schreiter, K., Mahr, K., Niederweis, M., . . . . Fey, G. H. (2004). A recombinant bispecific single-chain Fv antibody against HLA class II and Fc c RIII (CD16) triggers effective lysis of lymphoma cells, 167-179. https://doi.org/10.1111/j.1365-2141.2004.04893.x
Burns, L. J., Weisdorf, D. J., Defor, T. E., Vesole, D. H., Repka, T. L., Blazar, B. R., & Burger, S. R. (2003). Immunotherapy IL-2-based immunotherapy after autologous transplantation for lymphoma and breast cancer induces immune activation and cytokine release: a phase I/II trial, 177-186. https://doi.org/10.1038/sj.bmt. 1704086
Byrne, H., Conroy, P. J., Whisstock, J. C., & Kennedy, R. J. O. (2013). A tale of two specificities: bispecific antibodies for therapeutic and diagnostic applications. Trends in Biotechnology, 1-12. https://doi.org/10.1016/j.tibtech.2013.08.007 Caligiuri, M. A. (2019). Human natural killer cells, 112(3), 461-470. https://doi.org/10.1182/blood2007-09-077438.
Carlos, L., & Carneiro, J. (2003). Basic Histology, 7580. https://doi.org/10.1111/j. 1469-7580.2007.00771.x
Carson, W. E., Baumann, H., & Caligiuri, M. A. (1997). A potential role for interleukin-15 in the regulation of human natural killer cell survival. Find the latest version: The Journal of Clininal Investigation, 99(5), 937-943.
Chan, A. C., & Carter, P. J. (2010). Therapeutic antibodies for autoimmunity and inflammation. Nature Publishing Group, 10(5), 301-316. https://doi.org/10.1038/nri2761
Connor, B. P. O., Raman, V. S., Erickson, L. D., Cook, W. J., Weaver, L. K., Ahonen, C., . . . . Noelle, R. J. (2004). BCMA Is Essential for the Survival of Long-lived Bone Marrow Plasma Cells, 199(1). https://doi.org/10.1084/jem.20031330
Correia, I., Sung, J., Burton, R., Jakob, C. G., Ghayur, T., Radziejewski, C., . . . . Radziejewski, C. (2013). The structure of dual-variable-domain immunoglobulin molecules alone and bound to antigen, 862. https://doi.org/10.4161/mabs.24258
Elliott, N. T., & Yuan, F. A. N. (2011). A Review of Three-Dimensional In Vitro Tissue Models for Drug Discovery and Transport Studies. Journal of Pharmaceutical Sciences, 100(1), 59-74. https://doi.org/10.1002/jps.22257
Engelhardt, M., Terpos, E., Kleber, M., Gay, F., Wasch, R., Morgan, G., . . . . Brighen, S. (2014). European Myeloma Network recommendations on the evaluation and treatment of newly diagnosed patients with multiple myeloma Haematologica. https://doi.org/10.3324/haematol.2013.099358
Fang, D., Nguyen, T. K., Leishear, K., Finko, R., Kulp, A. N., Hotz, S., . . . . Herlyn, M. (2005). A Tumorigenic Subpopulation with Stem Cell Properties in Melanomas, (20), 9328-9338. https://doi.org/10.1158/0008-5472.CAN-05-1343
Fong, C. (2016). Permeability of the blood brain barrier: molecular mechanism of transport of drugs and important compounds To cite this version: HAL Id: hal-physiologically 01326584. https://doi.org/10.1007/s00232-015-9778-9
Freud, A. G., Becknell, B., Roychowdhury, S., Mao, H. C., Ferketich, A. K., Nuovo, G. J., . . . . Caligiuri, M. A. (2005). A Human CD34 (+) Subset Resides in Lymph Nodes and Differentiates into CD56 bright Natural Killer Cells, 22, 295-304. https://doi.org/10.1016/j.immuni.2005.01.013
Fujisaki, H., Kakuda, H., Shimasaki, N., Imai, C., Ma, J., Lockey, T., . . . . Campana, D. (2009). Expansion of Highly Cytotoxic Human Natural Killer Cells for Cancer Cell Therapy, (9), 4010-4018. https://doi.org/10.1158/0008-5472.CAN-08-3712
Galandrini, R., De Maria, R., Piccoli, M., Frati, L., & Santoni, A. (1994). CD44 triggering enhances human NK cell cytotoxic functions. Journal of Immunology, 153(10), 4399-4407.
Gardiner, C. M. (2017). NK Cell exhaustion, 8 (June). https://doi.org/10.3389/fimmu.2017.00760
Gasteiger, G., Hemmers, S., Firth, M. A., Le, A., Huse, M., Sun, J. C., & Rudensky, A. Y. (2013). IL-2-dependent tuning of NK cell sensitivity for target cells is controlled by regulatory T cells, 210(6), 1167-1178. https://doi.org/10.1084/jem.20122462
Gehlert, C. L. (2018). Multifunktionale Fusionsproteine zur tumorzellabhängigen Expansion von NK-Zellen in der Behandlung von B-Zell-Neoplasien.
Giril, J. G., Ahdieh, M., Eisenman, J., Shanebeck, K., Grabstein, K., Kumaki, S., . . . . Cosman, D. (1994). Utilization of the β and γ chains of the IL-2 receptor by the novel cytokine IL-15, 13 (12), 2822-2830. https://doi.org/10.1002/j.1460-2075.1994.tb06576.x
Graziano, R. F., & Guyre, P. M. (2006). Antibody-dependent Cell-mediated Cytotoxicity (ADCC). Groth, A., Pogge, E., Strandmann, V., & Koehl, U. (2011). Mechanisms of Tumor and Viral Immune Escape from Natural Killer Cell-Mediated, 344-354. https://doi.org/10.1159/000327014
Hanahan, D., & Weinberg, R. A. (2011). Hallmarks of Cancer: The Next Generation. Cell, 144 (5), 646-674. https://doi.org/10.1016/j.cell.2011.02.013
Haurowitz, F., & Schwerin, P. (1942). The valence of antibodies and the structure of the antigenantibody precipitate., 146-150.
Harris F, Berdugo Y A, Tree T. IL-2-based approaches to Treg enhancement. Clin Exp Immunol. 2023 Mar. 16; 211(2):149-163. doi: 10.1093/cei/uxac105.
Hedman, K., Lappalainen, M., Söderlund, M., & Hedman, L. (1993). Avidity of IgG in serodiagnosis of infectious diseases.
Heinrich, P. C., Müller, M., & Graeve, L. (2014). Löffler/Petrides Biochemie und Pathobiochemie. Springer-Verlag.
Herberman, R. B., Nunn, M. E., Holden, H. T., & Lavrin, D. H. (1975). ‘Natural cytotoxic reactivity of mouse lymphoid cells against syngeneic and allogeneic tumors. II. Characterization of effector cells’. International Journal of Cancer, 16(2), 230-239.
Hu, Y., Tian, Z., & Zhang, C. (2019). Natural Killer Cell-Based Immunotherapy for Cancer: Advances and Prospects. Engineering, 5(1), 106-114. https://doi.org/10.1016/j.eng.2018.11.015
Johnson, S., Burke, S., Huang, L., Gorlatov, S., Li, H., Wang, W., . . . . Bonvini, E. (2010). Effector Cell Recruitment with Novel Fv-based Dual-affinity Re-targeting Protein Leads to Potent Tumor Cytolysis and in Vivo B-cell Depletion. Journal of Molecular Biology, 399(3), 436-449. https://doi.org/10.1016/j.jmb.2010.04.001
Kermer V, Hornig N, Harder M, Bondarieva A, Kontermann R E, Müller D. Combining antibody-directed presentation of IL-15 and 4-1BBL in a trifunctional fusion protein for cancer immunotherapy. Mol Cancer Ther. 2014 January; 13(1):112-21. doi: 10.1158/1535-7163.MCT-13-0282. Epub 2013 Nov. 6.
Kiessling, R., Klein, E., & Wigzell, H. (1975). ‘Natural’ killer cells in the mouse. I. Cytotoxic cells with specificity for mouse Moloney leukemia cells. Specificity and distribution according to genotype”. International Journal of Cancer, 5(2), 112-117.
Klein, C., Lammens, A., Schäfer, W., Georges, G., Schwaiger, M., Mössner, E., . . . . Niederfellner, G. (2013). Epitope interactions of monoclonal antibodies targeting CD20 and their relationship to functional properties, 5(1), 22-33.
Kneitz, R. (2004). Untersuchung der Avidität von virusspezifischen Antikörpern in Serum und Liquor bei Patienten mit Virusenzephalitiden und Multipler Sklerose Inaugural-Dissertation.
Köhler, G., & Milstein, C. (1975). Continuous cultures of fused cells secreting antibody of predefined specificity. Nature, 256, 495-497.
Laabi, Y., Gras, M., Brouet, J., Berger, R., Larsen, C., Tsapis, A., . . . . Louis, H. Saint. (1994). The BCMA gene, preferentially expressed during B lymphoid maturation, is bidirectionally transcribed, 22(7), 1147-1154.
Lanier, L. L. (2015). NKG2D Receptor and Its Ligands in Host Defense. Cancer Immunology, Immunotherapy, 3(6), 575-583. https://doi.org/10.1158/2326-6066.CIR-15-0098
Li, B., Zhao, L., Guo, H., Wang, C., Zhang, X., Wu, L., . . . . Guo, Y. (2016). Characterization of a rituximab variant with potent antitumor activity against rituximab-resistant B-cell lymphoma, 114(24), 5007-5016. https://doi.org/10.1182/blood-2009-06-225474. The
Ljunggren, H. G., & Karre, K. (1985). HOST RESISTANCE DIRECTED SELECTIVELY H-2-DEFICIENT LYMPHOMA, 162 (December), 1745-1759.
Maloney, B. D. G., Grillo-lo, A. J., White, C. A., Bodkin, D., Schilder, R. J., Neidhart, J. A., . . . . Levy, R. (1997). IDEC-C2B8 (Rituximab) Anti-CD20 Monoclonal Antibody Therapy in Patients With Relapsed Low-Grade Non-Hodgkin's Lymphoma. Blood, 90(6), 2188-2195.
Mamessier, E., Moretta, A., Olive, D., Mamessier, E., Sylvain, A., Thibult, M., . . . . Olive, D. (2011). Human breast cancer cells enhance self tolerance by promoting evasion from NK cell antitumor immunity Find the latest version: Human breast cancer cells enhance self tolerance by promoting evasion from NK cell antitumor immunity, 121(9), 3609-3622. https://doi.org/10.1172/JCI45816.NK
Mandal, A., & Viswanathan, C. (2015). Natural killer cells: In health and disease. Hematology/Oncology and Stem Cell Therapy, 8(2), 47-55. https://doi.org/10.1016/j.hemonc.2014.11.006
Mason, D. W., & Williams, A. F. (1980). The Kinetics of Antibody Binding to Membrane Antigens in Solution and at the Cell Surface, 187, 1-20.
Mice, L., Gorellk, E., Rosen, B., Copeland, D., Weatherly, B., & Herberman, R. B. (1984). Evaluation of Role of Natural Killer Cells in Radiation-Induced Leukemogenesis in Mice, 72(6), 1397-1403.
Miller, J. S., Soignier, Y., Panoskaltsis-mortari, A., Mcnearney, S. A., Yun, G. H., Fautsch, S. K., . . . Mcglave, P. B. (2005). Successful adoptive transfer and in vivo expansion of human haploidentical NK cells in patients with cancer, 105(8), 3051-3058. https://doi.org/10.1182/blood-2004-07-2974.Supported
Moretta, A., Bottino, C., Vitale, M., Pende, D., Biassoni, R., Mingari, M. C., & Moretta, L. (1996). RECEPTORS FOR HLA CLASS-I MOLECULES IN HUMAN NATURAL KILLER CELLS. Annual Review of Immunology, 14, 619-648.
Mu, K. M., Arndt, K. M., & Plu, A. (1998). Model and Simulation of Multivalent Binding to Fixed Ligands, 158, 149-158.
Nezlin, R. (1998). General characteristics of immunoglobulin molecules. The Immunoglobulins: Structure and Function. Academic, 3-73.
Onoyama, H., Saitoh, Y., & Miyazawa, M. (1998). Tumor Imaging, 2431-2435.
Orange, J. S. (2008). Formation and function of the lytic NK-cell immunological synapse. Nature Reviews Immunology, 8.
Oyer, J. L., Igarashi, R. Y., Kulikowski, A. R., Colosimo, D. A., Solh, M. M., Zakari, A., . . . . Copik, A. J. (2015). Generation of Highly Cytotoxic Natural Killer Cells for Treatment of Acute Myelogenous
Leukemia Using a Feeder-Free, Particle-Based Approach. Biology of Blood and Marrow Transplantation, 21(4), 632-639. https://doi.org/10.1016/j.bbmt.2014.12.037 Pardridge, W. M. (2012). Drug transport across the blood-brain barrier, 32(11), 1959-1972. https://doi.org/10.1038/jcbfm.2012.126
Pawluczkowycz, A. W., Beurskens, F. J., Paul, V., Lindorfer, M. A., Winkel, J. G. J. Van De, Parren, W. H. I., . . . . Taylor, R. P. (2019). Binding of Submaximal Clq Promotes Complement Dependent Cytotoxicity (CDC) of B Cells Opsonized with Anti-CD20 mAbs Ofatumumab (OFA) or Rituximab (RTX): Considerably Higher Levels of CDC Are Induced by OFA than by RTX, (Cdc). https://doi.org/10.4049/jimmunol.0900632
Peipp, M., & Valerius, T. (2002). Bispecific antibodies targeting cancer cells, 507-511.
Pérez-Villar, J. J., Melero, I., Navarro, F., Carretero, M., Bellón, T., Llano, M., . . . . López-Botet, M. (1997). The CD94/NKG2-A inhibitory receptor complex is involved in natural killer cell-mediated recognition of cells expressing HLA-G1. Journal of Immunology, 158(12), 5736-5743.
Przewoznik, M. (2011). Thema der Dissertation: Phänotypische und funktionelle Charakterisierung von natürlichen Killerzellen in einem murinen.
Riccardi, C., Santoni, A., & Barlozzari, T. (1980). IN VIVO NATURAL REACTIVITY OF MICE AGAINST TUMOR CELLS, 486, 475-486.
Romee R, Schneider S E, Leong J W, Chase J M, Keppel C R, Sullivan R P, Cooper M A, Fehniger T A. Cytokine activation induces human memory-like NK cells. Blood. 2012 Dec. 6; 120(24):4751-60. doi: 10.1182/blood-2012-04-419283. Epub 2012 Sep. 14.
Romee R, Rosario M, Berrien-Elliott M M, Wagner J A, Jewell B A, Schappe T, Leong J W, Abdel-Latif S, Schneider S E, Willey S, Neal C C, Yu L, Oh S T, Lee Y S, Mulder A, Claas F, Cooper M A, Fehniger T A. Cytokine-induced memory-like natural killer cells exhibit enhanced responses against myeloid leukemia. Sci Transl Med. 2016 Sep. 21; 8(357):357ra123. doi: 10.1126/scitranslmed.aaf2341.
Ritz, J. (2006). Trans-presentation of IL-15. Blood, 108(7), 2135-2136.
Roschewski, M. J., & Wilson, W. H. (2014). Non-Hodgkin Lymphoma. In Abeloff's Clinical Oncology (Fifth Edition) (pp. 2033-2059).
Scott, A. M., Wolchok, J. D., & Old, L. J. (2012). Antibody therapy of cancer. Nature Reviews Cancer, 12(4), 1474-1768.
Segal, D. M., Weiner, G. J., & Weiner, L. M. (1999). Bispecific antibodies in cancer therapy, (c), 558-562.
Smyth, M. J., Cretney, E., Kelly, J. M., Westwood, J. A., Street, S. E. A., Yagita, H., . . . . Hayakawaa, Y. (2005). Activation of NK cell cytotoxicity. Molecular Immunology, 42(4), 501-510.
Spiess, C., Zhai, Q., & Carter, P. J. (2015). Alternative molecular formats and therapeutic applications for bispecific antibodies ζ. Molecular Immunology, 67(2), 95-106. https://doi.org/10.1016/j.molimm.2015.01.003
Strandmann, E. P. Von, Simhadri, V. R., Tresckow, B. Von, Sasse, S., Simhadri, V. L., Borchmann, P., . . . Engert, A. (2007). Human Leukocyte Antigen-B-Associated Transcript 3 Is Released from Tumor Cells and Engages the NKp30 Receptor on Natural Killer Cells, (December), 965-974. https://doi.org/10.1016/j.immuni.2007.10.010
Tai, Y. (2018). Targeting B Cell Maturation Antigen (BCMA) in Multiple Myeloma: Potential Uses of BCMA-Based immunotherapy, 9 (August). https://doi.org/10.3389/fimmu.2018.01821
Tai, Y., Dillon, M., Song, W., Leiba, M., Li, X., Burger, P., . . . . Elisa, C. S. (2008). Anti-CS1 humanized monoclonal antibody HuLuc63 inhibits myeloma cell adhesion and induces antibodydependent cellular cytotoxicity in the bone marrow milieu. Blood, 112(4), 1329-1338. https://doi.org/10.1182/blood-2007-08-107292. The
Tedder, T. F., Streuli, M., Schlossman, S. F., & Saito, H. (1988). Isolation and structure of a cDNA encoding the B1 (CD20) cell-surface antigen of human B lymphocytes, 85 (January), 208-212.
Terme, M., Ullrich, E., Delahaye, N. F., Chaput, N., & Zitvogel, L. (2008). Natural killer cell-directed therapies: moving from unexpected results to successful strategies, 9(5), 486-494. https://doi.org/10.1038/ni1580
Thie, H. (2008). Antikörpervarianten mit verbesserter Affinität.
Tsang, R. W., & Gospodarowicz, M. K. (2012). Non-Hodgkin's Lymphoma. In Clinical Radiation Oncology (Third Edition) (pp. 1545-1572).
Vitale, B. M., Bottino, C., Sivori, S., Sanseverino, L., Castriconi, R., Marcenaro, E., . . . . Moretta, A. (1998). NKp44, a Novel Triggering Surface Molecule Specifically Expressed by Activated Natural Killer Cells, Is Involved in Non-Major Histocompatibility Complex-restricted TumorCell Lysis, 187(12).
Vivier, E., Raulet5, D. H., Moretta6, A., Caligiuri, M. A., Zitvogel, L., & Lanier, L. L. (2011). Innate or Adaptive Immunity? The Example of Natural Killer Cells. Science, 331(6013), 44-49.
Wagner, E. (2010). Generierung muriner monoklonaler Antikörper und gentechnische Herstellung spezifischer F ab-Fragmente gegen die tumorassoziierte Isoform 1 des humanen Tenascin C für die Radioimmuntherapie von Gliomen.
Wagner, J., Pfannenstiel, V., & Waldmann, A. (2017). A Two-Phase Expansion Protocol Combining Interleukin (IL)-15 and IL-21 Improves Natural Killer Cell Proliferation and Cytotoxicity against Rhabdomyosarcoma, 8 (June). https://doi.org/10.3389/fimmu.2017.00676
Waldmann, T. A. (2006). The biology of interleukin-2 and interleukin-15: implications for cancer therapy and vaccine design. Nature Reviews Immunology, 6(8).
Watanabe, M., Wallace, P. K., Keler, T., Deo, Y. M., Akewanlop, C., & Hayes, D. F. (1999). Antibody dependent cellular phagocytosis (ADCP) and antibody dependent cellular cytotoxicity (ADCC) of breast cancer cells mediated by bispecific antibody, MDX-210, 199-207.
Weers, M. De, Tai, Y., Veer, M. S. Van Der, Bakker, J. M., Vink, T., Jacobs, D. C. H., . . . . Parren, P. W. H. I. (2011). Daratumumab, a Novel Therapeutic Human CD38 Monoclonal Antibody, Induces Killing of Multiple Myeloma and Other Hematological Tumors. The Journal of Immunology. https://doi.org/10.4049/jimmunol.1003032
Wörmann, B. (2018). Multiples Myelom

1. A fusion protein comprising
(a) an antibody or an antibody fragment binding to an antigen being expressed on the surface of a target cell of NK cells, T cells and/or NKT cells, preferably the surface of a B cell or a tumor cell,
(b) an antibody or an antibody fragment binding to 4-1BB, NKG2D, NKp30, NKp46, NKp44, 2B4, CD28 or DNAM1, and
(c) IL-15, IL-2, IL-18, IL-21 or IL-12.

2. The fusion protein of claim 1, wherein the antigen of (a) is selected from the group consisting of CD20, BCMA, CD19, CD22, CD37, CD38, CD7, CD33, CD44, CD54, CD64, CD75s, CD79b, CD96, CD123, CD317, CD319, FCRL5, EGFR, B7-H3, HER2, EpCAM, CEA, GD2 and Claudin 6/18, ROR1, Trop-2, PSMA, FolR1, STEAP1, Her3, uPAR, Muc-1, cMet, CXCR4, SAP-1, Muc-16, TAG-72, HLA-DR, CD30, DLL4, CD221, Mesothelin, GPRC5D, Nectin-4, LIV-1, and Tissue factor and is preferably CD20 or BCMA.

3. The fusion protein of claim 1 or 2, wherein each of the antibody fragment of (a) and (b) is independently selected from Fab, scFv, Fv, VHH and dAb, and wherein the antibody fragment of (a) is preferably Fab and the antibody fragment of (b) is preferably scFv.

4. The fusion protein of any one of claims 1 to 3, wherein (a), (b) and/or (c) are fused to each other by a flexible linker, preferably a flexible peptide linker and most preferably a flexible peptide linker of at least 5 amino acids.

5. The fusion protein of any one of claims 1 to 4, wherein (a) is at the N-terminus, (b) is between (a) and (c), and (c) is at the C-terminus of the fusion protein.

6. The fusion protein of any one of claims 1 to 5, wherein the fusion protein further comprises a purification tag, preferably a His-tag or a myc-tag.

7. The fusion protein of any one of claims 1 to 6, wherein the antigen of (a) 4-1BB, NKG2D, NKp30, NKp46, NKp44, 2B4, CD28 or DNAM-1, of (b) IL-15, IL-2, IL-18, IL-21 or IL-12 and/or of (c) are human.

8. The fusion protein of any one of claims 1 to 6, wherein (c) comprises IL-15 fused to the sushi domain of the IL-15 receptor.

9. A nucleic acid molecule, a set of nucleic acid molecules, an expression vector or a set of expression vectors encoding the fusion protein of any one of claims 1 to 8.

10. A host cell, preferably a non-human host cell comprising the nucleic acid molecule, set of nucleic acid molecules, the expression vector or set of expression vectors of claim 9.

12. A method for producing the fusion protein of any one of claims 1 to 8 comprising
(a) culturing the host cell of claim 10 under conditions where the host cell expresses the fusion protein of any one of claims 1 to 8, and
(b) isolating the fusion protein of any one of claims 1 to 8 as expressed in (a).

13. A composition, preferably a pharmaceutical composition or a kit comprising the fusion protein of any one of claims 1 to 8, the nucleic acid sequence, set of nucleic acid molecules, expression vector or set of expression vectors of claim 9, or the host cell of claim 10.

14. The fusion protein of any one of claims 1 to 8, the nucleic acid sequence, a set of nucleic acid molecules, expression vector, or set of expression vectors of claim 9, or the host cell of claim 10 optionally in combination with CAR NK-cells or CAR T-cells for use in the treatment of a tumor.

15. A method for the ex vivo or in vitro expansion of NK-cells, T cells and/or NTK cells comprising
(a) coculturing NK-cells, T cells and/or NTK cells with target cells of NK cells, T cells and/or NTK cells, preferably B cells or tumor cells in the presence of the fusion protein of any one of claims 1 to 8, the nucleic acid sequence, set of nucleic acid molecules, expression vector or set of expression vectors of claim 9, or the host cell of claim 10,
(b) optionally purifying or isolating the expanded NK-cells, T cells and/or NTK cells as obtained in step (a) from the coculture.


SEQUENCE LISTING (80 sequences)

SEQ ID NO: 1 - DNA, 639 bp, synthetic construct
  source 1..639: mol_type = other DNA; note = RTX Light Chain
caaatcgtcctcagccagtctccagccatcctctccgcatctcctggcgagaaggtgaccatgacctgcagagcttccagctccgtcagctacatccactggttccagcagaagcctggaagcagcccaaagccctggatctacgccaccagcaacctggccagcggagtcccagtgaggttctcagggagcggaagtggcacctcctacagcctgaccatcagcagggtggaggctgaagatgcagccacctactactgccagcagtggacctccaatcctcccacctttggtggaggaaccaagcttgagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 2 - protein, 213 aa, synthetic construct
  source 1..213: note = RTX Light Chain
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 363 bp, synthetic construct
  source 1..363: mol_type = other DNA; note = RTX-VH
caggtacaactgcagcagcctggagccgagttggtgaagcctggcgccagcgtcaaaatgagctgcaaagcctcaggctacaccttcaccagctacaacatgcactgggtgaagcagacaccaggacgaggactggagtggatcggcgctatctaccctggcaacggagacacctcctacaaccagaagttcaaggggaaggcaacactgaccgctgacaagtccagcagcacagcctacatgcagctgagctccctgacttccgaggatagcgctgtgtactactgcgcaaggtccacctactacggaggcgactggtacttcaacgtgtggggcgccggaactaccgtgactgtgagcagc

SEQ ID NO: 4 - protein, 121 aa, synthetic construct
  source 1..121: note = RTX-VH
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSS

SEQ ID NO: 5 - DNA, 642 bp, synthetic construct
  source 1..642: mol_type = other DNA; note = BCMA Light Chain
gacatccagatgacccagagccctagctcactgagcgccagcgtgggcgacagggtgaccattacctgctccgccagccaggacatcagcaactacctgaactggtaccagcagaagcccggcaaggcccccaagctgctgatctactacacctccaacctgcactccggcgtgcccagcaggttcagcggaagcggcagcggcaccgatttcaccctgaccatctccagcctgcagcccgaggacttcgccacctactactgccagcagtacaggaagctcccctggactttcggccagggcaccaagcttgagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 6 - protein, 214 aa, synthetic construct
  source 1..214: note = BCMA Light Chain
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - DNA, 363 bp, synthetic construct
  source 1..363: mol_type = other DNA; note = BCMA-VH
caggtgcagctggtccagagcggcgccgaagtgaagaagcccggcagctccgtgaaagtgagctgcaaggccagcggcggcaccttcagcaactactggatgcactgggtgaggcaggcccccggacagggcctggagtggatgggcgccacctacaggggccacagcgacacctactacaaccagaagttcaagggccgggtgaccatcaccgccgacaagagcaccagcaccgcctacatggaactgagcagcctcaggagcgaggacaccgctgtgtattactgcgccaggggcgccatctacgacggctacgacgtgctggacaactggggccagggcacactagtgaccgtgtccagc

SEQ ID NO: 8 - protein, 121 aa, synthetic construct
  source 1..121: note = BCMA-VH
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 9 - DNA, 330 bp, synthetic construct
  source 1..330: mol_type = other DNA; note = CD137-VL
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggcgctcactttcggcggagggaccaaggtggagatcaaacga

SEQ ID NO: 10 - protein, 110 aa, synthetic construct
  source 1..110: note = CD137-VL
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKR

SEQ ID NO: 11 - DNA, 363 bp, synthetic construct
  source 1..363: mol_type = other DNA; note = CD137-VH
caggtgcaactacagcagtggggcgcaggactgttgaagccttcggagaccctgtccctcacctgcgctgtctatggtgggtccttcagtggttactactggagctggatacgccagtccccagagaaggggctggagtggattggggaaatcaatcatggtggatacgtcacctacaatccgtccctggagagtcgagtcaccatatcagtagacacgtccaagaaccagttctccctgaagctgagctctgtgaccgccgcggacacggctgtatattactgtgcgagggactatggtccggggaattatgactggtacttcgatctctggggccgtggcaccctggtcactgtctcctcg

SEQ ID NO: 12 - protein, 121 aa, synthetic construct [sequence omitted]

SEQ ID NO: 13 - protein, 52 aa, synthetic construct
  source 1..52: note = Flexible linker 1
GGGGSGGGGSGGGGSGGGGSAAQPAGSSASGAGGGGSGGGGSGGGGSGGGGS

SEQ ID NO: 14 - protein, 21 aa, synthetic construct
  source 1..21: note = Flexible linker 2
GGGGSGGGGSGGGGSAQPAMA

SEQ ID NO: 15 - protein, 20 aa, synthetic construct
  source 1..20: note = Flexible linker 3
GGGGSGGGGSGGGGSGGGGS

SEQ ID NO: 16 - protein, 19 aa, synthetic construct
  source 1..19: note = Flexible linker 4
ASGAGGGGSGGGGSGGGGS

SEQ ID NO: 17 - protein, 20 aa, synthetic construct
  source 1..20: note = Flexible linker 5
GGGGSGGGGSGGGGSGGGGS

SEQ ID NO: 18 - DNA, 156 bp, synthetic construct
  source 1..156: mol_type = other DNA; note = Flexible linker 1
ggaggaggcggatctggtggaggaggatcaggaggaggcggatcaggaggtggcggaagtgcggcccagccggccggttcttcggcctcgggggccggaggtggcggttctggtggtggcggatcaggaggaggcggatcaggaggcggaggatct

SEQ ID NO: 19 - DNA, 63 bp, synthetic construct
  source 1..63: mol_type = other DNA; note = Flexible linker 2
ggaggaggcggatctggtggaggaggatcaggaggaggcggatcggcccagccggccatggcg

SEQ ID NO: 20 - DNA, 60 bp, synthetic construct
  source 1..60: mol_type = other DNA; note = Flexible linker 3
ggtggtggtggttctggtggtggtggttctggcggcggcggctccggtggtggtggatcc

SEQ ID NO: 21 - DNA, 57 bp, synthetic construct
  source 1..57: mol_type = other DNA; note = Flexible linker 4
gcctcgggggccggtggtggtggttctggcggcggcggctccggtggtggtggatcc

SEQ ID NO: 22 - DNA, 60 bp, synthetic construct; identical to SEQ ID NO: 20
  source 1..60: mol_type = other DNA; note = Flexible linker 5

SEQ ID NO: 23 - protein, 6 aa, synthetic construct
  source 1..6: note = His-tag
HHHHHH

SEQ ID NO: 24 - DNA, 18 bp, synthetic construct
  source 1..18: mol_type = other DNA; note = His-tag
catcatcatcatcatcat

SEQ ID NO: 25 - protein, 11 aa, synthetic construct
  source 1..11: note = Myc-tag
EQKLISEEDLN

SEQ ID NO: 26 - DNA, 33 bp, synthetic construct
  source 1..33: mol_type = other DNA; note = Myc-tag
gaacaaaaactcatctcagaagaggatctgaat

SEQ ID NO: 27 - protein, 114 aa, Homo sapiens
  source 1..114: note = IL15
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 28 - DNA, 342 bp, Homo sapiens
  source 1..342: mol_type = genomic DNA; note = IL15
aactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 29 - protein, 77 aa, Homo sapiens
  source 1..77: note = IL15 sushi domain
ITCPPPMSVEHADIWVKSYSLYSRERYICNSGFKRKAGTSSLTECVLNKATNVAHWTTPSLKCIRDPALVHQRPAPP

SEQ ID NO: 30 - DNA, 231 bp, Homo sapiens
  source 1..231: mol_type = genomic DNA; note = IL15 sushi domain
atcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccc

SEQ ID NO: 31 - protein, 757 aa, synthetic construct
  source 1..757: note = RTX-CD137scFv-IL15
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSAQPAMAEIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRGGGGSGGGGSGGGGSGGGGSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASGAGGGGSGGGGSGGGGSITCPPPMSVEHADIWVKSYSLYSRERYICNSGFKRKAGTSSLTECVLNKATNVAHWTTPSLKCIRDPALVHQRPAPPGGGGSGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSRGGPEQKLISEEDLNSAVDHHHHHH

SEQ ID NO: 32 - DNA, 2271 bp, synthetic construct
  source 1..2271: mol_type = other DNA; note = RTX-CD137scFv-IL15
caggtacaactgcagcagcctggagccgagttggtgaagcctggcgccagcgtcaaaatgagctgcaaagcctcaggctacaccttcaccagctacaacatgcactgggtgaagcagacaccaggacgaggactggagtggatcggcgctatctaccctggcaacggagacacctcctacaaccagaagttcaaggggaaggcaacactgaccgctgacaagtccagcagcacagcctacatgcagctgagctccctgacttccgaggatagcgctgtgtactactgcgcaaggtccacctactacggaggcgactggtacttcaacgtgtggggcgccggaactaccgtgactgtgagcagcgcctccactaagggacctagcgttttcccactggctccttcctctaagagcacttccggtggaacagccgctctcggatgcctggtcaaggattacttcccagaacccgtgactgtgagctggaatagcggcgcattgacctcaggggtccacacattcccagccgtacttcagagttccggcctgtatagcctgtcctctgtcgtgaccgttcctagctcaagcctcggtacacagacctacatctgcaacgtgaaccataagcccagcaacaccaaggtggacaagaaagtcgaacccaaatcctgcgacaaaacgcacacaggaggaggcggatctggtggaggaggatcaggaggaggcggatcggcccagccggccatggcggaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggcgctcactttcggcggagggaccaaggtggagatcaaacgaggtggtggtggttctggtggtggtggttctggcggcggcggctccggtggtggtggatcccaggtgcaactacagcagtggggcgcaggactgttgaagccttcggagaccctgtccctcacctgcgctgtctatggtgggtccttcagtggttactactggagctggatacgccagtccccagagaaggggctggagtggattggggaaatcaatcatggtggatacgtcacctacaatccgtccctggagagtcgagtcaccatatcagtagacacgtccaagaaccagttctccctgaagctgagctctgtgaccgccgcggacacggctgtatattactgtgcgagggactatggtccggggaattatgactggtacttcgatctctggggccgtggcaccctggtcactgtctcctcggcctcgggggccggtggtggtggttctggcggcggcggctccggtggtggtggatccatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccacccggtggtggtggttctggtggtggtggttctggcggcggcggctccggtggtggtggatccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttctcgaggagggcccgaacaaaaactcatctcagaagaggatctgaatagcgccgtcgaccatcatcatcatcatcat

SEQ ID NO: 33 - protein, 757 aa, synthetic construct
  source 1..757: note = BCMA-CD137scFv-IL15
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSAQPAMAEIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRGGGGSGGGGSGGGGSGGGGSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASGAGGGGSGGGGSGGGGSITCPPPMSVEHADIWVKSYSLYSRERYICNSGFKRKAGTSSLTECVLNKATNVAHWTTPSLKCIRDPALVHQRPAPPGGGGSGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSRGGPEQKLISEEDLNSAVDHHHHHH

SEQ ID NO: 34 - DNA, 2271 bp, synthetic construct
  source 1..2271: mol_type = other DNA; note = BCMA-CD137scFv-IL15
caggtgcagctggtccagagcggcgccgaagtgaagaagcccggcagctccgtgaaagtgagctgcaaggccagcggcggcaccttcagcaactactggatgcactgggtgaggcaggcccccggacagggcctggagtggatgggcgccacctacaggggccacagcgacacctactacaaccagaagttcaagggccgggtgaccatcaccgccgacaagagcaccagcaccgcctacatggaactgagcagcctcaggagcgaggacaccgctgtgtattactgcgccaggggcgccatctacgacggctacgacgtgctggacaactggggccagggcacactagtgaccgtgtccagcgcctccactaagggacctagcgttttcccactggctccttcctctaagagcacttccggtggaacagccgctctcggatgcctggtcaaggattacttcccagaacccgtgactgtgagctggaatagcggcgcattgacctcaggggtccacacattcccagccgtacttcagagttccggcctgtatagcctgtcctctgtcgtgaccgttcctagctcaagcctcggtacacagacctacatctgcaacgtgaaccataagcccagcaacaccaaggtggacaagaaagtcgaacccaaatcctgcgacaaaacgcacacaggaggaggcggatctggtggaggaggatcaggaggaggcggatcggcccagccggccatggcggaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggcgctcactttcggcggagggaccaaggtggagatcaaacgaggtggtggtggttctggtggtggtggttctggcggcggcggctccggtggtggtggatcccaggtgcaactacagcagtggggcgcaggactgttgaagccttcggagaccctgtccctcacctgcgctgtctatggtgggtccttcagtggttactactggagctggatacgccagtccccagagaaggggctggagtggattggggaaatcaatcatggtggatacgtcacctacaatccgtccctggagagtcgagtcaccatatcagtagacacgtccaagaaccagttctccctgaagctgagctctgtgaccgccgcggacacggctgtatattactgtgcgagggactatggtccggggaattatgactggtacttcgatctctggggccgtggcaccctggtcactgtctcctcggcctcgggggccggtggtggtggttctggcggcggcggctccggtggtggtggatccatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccacccggtggtggtggttctggtggtggtggttctggcggcggcggctccggtggtggtggatccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttctcgaggagggcccgaacaaaaactcatctcagaagaggatctgaatagcgccgtcgaccatcatcatcatcatcat

SEQ ID NO: 35 - protein, 1038 aa, synthetic construct
  source 1..1038: note = RTX-DuoFab-CD137scFv-IL15
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSGGGGSAAQPAGSSASGAGGGGSGGGGSGGGGSGGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSAQPAMAEIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRGGGGSGGGGSGGGGSGGGGSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASGAGGGGSGGGGSGGGGSITCPPPMSVEHADIWVKSYSLYSRERYICNSGFKRKAGTSSLTECVLNKATNVAHWTTPSLKCIRDPALVHQRPAPPGGGGSGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSRGGPEQKLISEEDLNSAVDHHHHHH

SEQ ID NO: 36 - DNA, 3114 bp, synthetic construct
  source 1..3114: mol_type = other DNA; note = RTX-DuoFab-CD137scFv-IL15
caggtacaactgcagcagcctggagccgagttggtgaagcctggcgccagcgtcaaaatgagctgcaaagcctcaggctacaccttcaccagctacaacatgcactgggtgaagcagacaccaggacgaggactggagtggatcggcgctatctaccctggcaacggagacacctcctacaaccagaagttcaaggggaaggcaacactgaccgctgacaagtccagcagcacagcctacatgcagctgagctccctgacttccgaggatagcgctgtgtactactgcgcaaggtccacctactacggaggcgactggtacttcaacgtgtggggcgccggaactaccgtgactgtgagcagcgcctccactaagggacctagcgttttcccactggctccttcctctaagagcacttccggtggaacagccgctctcggatgcctggtcaaggattacttcccagaacccgtgactgtgagctggaatagcggcgcattgacctcaggggtccacacattcccagccgtacttcagagttccggcctgtatagcctgtcctctgtcgtgaccgttcctagctcaagcctcggtacacagacctacatctgcaacgtgaaccataagcccagcaacaccaaggtggacaagaaagtcgaacccaaatcctgcgacaaaacgcacacaggaggaggcggatctggtggaggaggatcaggaggaggcggatcaggaggtggcggaagtgcggcccagccggccggttcttcggcctcgggggccggaggtggcggttctggtggtggcggatcaggaggaggcggatcaggaggcggaggatctcaagttcaactgcagcagcctggagccgaattggtgaagcctggcgcaagcgtcaaaatgagctgcaaagcgagtggctacaccttcacctcctacaacatgcactgggtgaagcaaacaccaggacgaggactggagtggattggcgcaatctaccctggcaacggagacacgagctacaaccagaagttcaaggggaaagcaacactgaccgctgacaagagcagcagcacagcctacatgcagctgagctccctgacttccgaggattccgctgtgtactactgcgcacgctccacctattacggtggcgactggtacttcaacgtttggggcgccggaactacggtgactgtgagtagcgcgagcactaaggggccaagtgttttcccactggcaccgtcctctaagagcacttccggtggaacagcggctcttggatgcctggtcaaggattacttccccgaacccgtcacggtgagctggaatagcggcgcattgacctctggcgtgcacacattccctgccgttctgcaatcttccggcctctattccctgtcctctgtggtgaccgttccaagctcaagcctcgggacccaaacctacatctgcaacgtgaaccataagccgagcaacacgaaggtcgacaagaaggtggagcccaagagctgcgacaaaacgcacacaggaggaggcggatctggtggaggaggatcaggaggaggcggatcggcccagccggccatggcggaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggcgctcactttcggcggagggaccaaggtggagatcaaacgaggtggtggtggttctggtggtggtggttctggcggcggcggctccggtggtggtggatcccaggtgcaactacagcagtggggcgcaggactgttgaagccttcggagaccctgtccctcacctgcgctgtctatggtgggtccttcagtggttactactggagctggatacgccagtccccagagaaggggctggagtggattggggaaatcaatcatggtggatacgtcacctacaatccgtccctggagagtcgagtcaccatatcagtagacacgtccaagaaccagttctccctgaagctgagctctgtgaccgccgcggacacggctgtatattactgtgcgagggactatggtccggggaattatgactggtacttcgatctctggggccgtggcaccctggtcactgtctcctcggcctcgggggccggtggtggtggttctggcggcggcggctccggtggtggtggatccatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccacccggtggtggtggttctggtggtggtggttctggcggcggcggctccggtggtggtggatccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttctcgaggagggcccgaacaaaaactcatctcagaagaggatctgaatagcgccgtcgaccatcatcatcatcatcat

SEQ ID NO: 37 - protein, 1038 aa, synthetic construct
  source 1..1038: note = BCMA-DuoFab-CD137scFv-IL15
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSGGGGSAAQPAGSSASGAGGGGSGGGGSGGGGSGGGGSQVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSAQPAMAEIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRGGGGSGGGGSGGGGSGGGGSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASGAGGGGSGGGGSGGGGSITCPPPMSVEHADIWVKSYSLYSRERYICNSGFKRKAGTSSLTECVLNKATNVAHWTTPSLKCIRDPALVHQRPAPPGGGGSGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSRGGPEQKLISEEDLNSAVDHHHHHH

SEQ ID NO: 38 - DNA, 3114 bp, synthetic construct
  source 1..3114: mol_type = other DNA; note = BCMA-DuoFab-CD137scFv-IL15
caggtgcaactggtccagagcggcgccgaagtgaagaagcccggcagctccgtgaaagtgagctgcaaggccagcggcggcaccttcagcaactactggatgcactgggtgaggcaggcccccggacagggcctggagtggatgggcgccacctacaggggccacagcgacacctactacaaccagaagttcaagggccgggtgaccatcaccgccgacaagagcaccagcaccgcctacatggaactgagcagcctcaggagcgaggacaccgctgtgtattactgcgccaggggcgccatctacgacggctacgacgtgctggacaactggggccagggcacactagtgaccgtgtccagcgcctccactaagggacctagcgttttcccactggctccttcctctaagagcacttccggtggaacagccgctctcggatgtctggtcaaggattacttcccagaacccgtgactgtgagctggaatagcggcgcattgacctcaggggtccacacattcccagccgtacttcagagttccggcctgtatagcctgtcctctgtcgtgaccgttcctagctcaagcctcggtacacagacctacatctgcaacgtgaaccataagcccagcaacaccaaggtggacaagaaagtcgaacccaaatcctgcgacaaaacgcacacaggaggaggcggatctggtggaggaggatcaggaggaggcggatcaggaggtggcggaagtgcggcccagccggccggttcttcggcctcgggggccggaggtggcggttctggtggtggcggatcaggaggaggcggatcaggaggcggaggatctcaggtgcagctggtccagagcggcgccgaagtgaagaagcccggcagctccgtgaaagtgagctgcaaggccagcggcggcaccttcagcaactactggatgcactgggtgaggcaggcccccggacagggcctggagtggatgggcgccacctacaggggccacagcgacacctactacaaccagaagttcaagggccgggtgaccatcaccgccgacaagagcaccagcaccgcctacatggaactgagcagcctcaggagcgaggacaccgctgtgtattactgcgccaggggcgccatctacgacggctacgacgtgctggacaactggggccagggcacactagtgaccgtgtccagcgcctccactaagggacctagcgttttcccactggctccttcctctaagagcacttccggtggaacagccgctctcggatgcctggtcaaggattacttcccagaacccgtgactgtgagctggaatagcggcgcattgacctcaggggtccacacattcccagccgtacttcagagttccggcctgtatagcctgtcctctgtcgtgaccgttcctagctcaagcctcggtacacagacctacatctgcaacgtgaaccataagcccagcaacaccaaggtggacaagaaagtcgaacccaaatcctgcgacaaaacgcacacaggaggaggcggatctggtggaggaggatcaggaggaggcggatcggcccagccggccatggcggaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggcgctcactttcggcggagggaccaaggtggagatcaaacgaggtggtggtggttctggtggtggtggttctggcggcggcggctccggtggtggtggatcccaggtgcaactacagcagtggggcgcaggactgttgaagccttcggagaccctgtccctcacctgcgctgtctatggtgggtccttcagtggttactactggagctggatacgccagtccccagagaaggggctggagtggattggggaaatcaatcatggtggatacgtcacctacaatccgtccctggagagtcgagtcaccatatcagtagacacgtccaagaaccagttctccctgaagctgagctctgtgaccgccgcggacacggctgtatattactgtgcgagggactatggtccggggaattatgactggtacttcgatctctggggccgtggcaccctggtcactgtctcctcggcctcgggggccggtggtggtggttctggcggcggcggctccggtggtggtggatccatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccacccggtggtggtggttctggtggtggtggttctggcggcggcggctccggtggtggtggatccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttctcgaggagggcccgaacaaaaactcatctcagaagaggatctgaatagcgccgtcgaccatcatcatcatcatcat

SEQ ID NO: 39 - protein, 5 aa, synthetic construct
  source 1..5: note = Truncated hinge
DKTHT

SEQ ID NO: 40 - DNA, 15 bp, synthetic construct
  source 1..15: mol_type = other DNA; note = Truncated hinge
gacaaaacgcacaca

SEQ ID NO: 41 - protein, 103 aa, synthetic construct
  source 1..103: note = CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 42 - DNA, 309 bp, synthetic construct
  source 1..309: mol_type = other DNA; note = CH1
gcctccactaagggacctagcgttttcccactggctccttcctctaagagcacttccggtggaacagccgctctcggatgcctggtcaaggattacttcccagaacccgtgactgtgagctggaatagcggcgcattgacctcaggggtccacacattcccagccgtacttcagagttccggcctgtatagcctgtcctctgtcgtgaccgttcctagctcaagcctcggtacacagacctacatctgcaacgtgaaccataagcccagcaacaccaaggtggacaagaaagtcgaacccaaatcctgc

SEQ ID NO: 43 - protein, 5 aa, synthetic construct
  source 1..5: note = Spacer1
SRGGP

SEQ ID NO: 44 - DNA, 15 bp, synthetic construct
  source 1..15: mol_type = other DNA; note = Spacer1
tctcgaggagggccc

SEQ ID NO: 45 - protein, 4 aa, synthetic construct
  source 1..4: note = Spacer2
SAVD

SEQ ID NO: 46 - DNA, 12 bp, synthetic construct
  source 1..12: mol_type = other DNA; note = Spacer2
agcgccgtcgac

SEQ ID NO: 47 - DNA, 657 bp, synthetic construct
  source 1..657: mol_type = other DNA; note = CD19 light chain
gatattgtgatgactcagtctccagccaccctgtctttgtctccaggggaaagagccaccctctcctgcaggtctagtaagagtctccagaatgtcaatggcaacacttacttgtattggttccagcagaagccaggccagtctcctcagctcctgatatatcggatgtccaaccttaattcaggagtcccagacaggttcagtggcagtgggtcaggaactgaattcacactgacaatcagtagtctggagcctgaagattttgcagtgtattactgtatgcaacatctggaatatccgatcacgttcggtgctgggaccaagcttgagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 48 - protein, 219 aa, synthetic construct
  source 1..219: note = CD19 light chain
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - DNA, 363 bp, synthetic construct
  source 1..363: mol_type = other DNA; note = CD19-VH
gaggtgcagctggtggagtctgggggaggcttggtaaagcctggggggtctcttaaactctcctgtgcagcctctggatatactttcactagctatgttatgcactgggtccgccaggctccagggaaggggctggagtggattggatatattaatccttacaatgatggtactaagtacaatgagaagttccagggcagagtcaccatttcctccgacaaatccattagcacagcctacatggagctgagcagcctgagatctgaggacacggccatgtattactgtgcaagagggacttattactacggtaccagggtatttgactactggggccaaggcactctcgtcaccgtctcctcg

SEQ ID NO: 50 - protein, 121 aa, synthetic construct
  source 1..121: note = CD19-VH
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 51 - DNA, 321 bp, synthetic construct
  source 1..321: mol_type = other DNA; note = NKp46-VL
gacattcagatgacccaatcccctgcttctctctcagcctctgtgggagagactgtgaccataacctgtcgtgttagcgagaacatctactcctatctcgcctggtatcagcagaaacaggggaaatccccacaactgctcgtgtacaatgccaagactctggcagaaggagtgccaagccgcttttccgggtctgggtctgggacacagttctcactgaagatcaactctttgcaacctgaggattttggctcttactactgtcagcatcactatggcacaccatggacgtttggtggcggcactaagctggagattaag

SEQ ID NO: 52 - protein, 107 aa, synthetic construct
  source 1..107: note = NKp46-VL
DIQMTQSPASLSASVGETVTITCRVSENIYSYLAWYQQKQGKSPQLLVYNAKTLAEGVPSRFSGSGSGTQFSLKINSLQPEDFGSYYCQHHYGTPWTFGGGTKLEIK

SEQ ID NO: 53 - DNA, 366 bp, synthetic construct
  source 1..366: mol_type = other DNA; note = NKp46-VH
gaggtacagttgcaggagagtgggcctggactggtcaaaccctcccaatctctgagcttgacatgcacagtcacaggctacagcatcacctccgactacgcttggaattggattcgacagtttcccggcaacaagctggaatggatgggctacatcacctatagtggtagcacttcctataatccctcacttgagagccggatttccatcactagggatacgagcaccaaccagttcttcctgcagctgaatagcgtcaccaccgaagatactgccacctattactgcgcaagaggcggttactatggcagttcatggggtgtattcgcctattggggacaggggacacttgtgacagtgtctgct

SEQ ID NO: 54 - protein, 122 aa, synthetic construct
  source 1..122: note = NKp46-VH
EVQLQESGPGLVKPSQSLSLTCTVTGYSITSDYAWNWIRQFPGNKLEWMGYITYSGSTSYNPSLESRISITRDTSTNQFFLQLNSVTTEDTATYYCARGGYYGSSWGVFAYWGQGTLVTVSA

SEQ ID NO: 55 - DNA, 324 bp, synthetic construct
  source 1..324: mol_type = other DNA; note = NKG2D-VL
tcttctgagctgactcaggaccctgctgtgtctgtggccttgggacagacagtcaggatcacatgccaaggagacagcctcagaggctattatgcaagctggtaccagcagaagccaggacaggcccctgtacttgtcacctatggtaacaacaagcggccctcaggaatcccagaccgattctctggctccagttcaggaaacacagcttccttgaccatcactgggactcaggcggaagatgaggctgactactattgtaactcccgggacaacaatggtaacttcgttatattcggcggagggaccaagctgaccgtcctg

SEQ ID NO: 56 - protein, 108 aa, synthetic construct
  source 1..108: note = NKG2D-VL
SSELTQDPAVSVALGQTVRITCQGDSLRGYYASWYQQKPGQAPVLVTYGNNKRPSGIPDRFSGSSSGNTASLTITGTQAEDEADYYCNSRDNNGNFVIFGGGTKLTVL

SEQ ID NO: 57 - DNA, 381 bp, synthetic construct
  source 1..381: mol_type = other DNA; note = NKG2D-VH
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgaaagatctctatgattacgtttgggggagttatcgccctaactggtacttcgatctctggggccgtggcaccctggtcactgtctcctca

SEQ ID NO: 58 - protein, 127 aa, synthetic construct
  source 1..127: note = NKG2D-VH
QVQLVQSGAEVKKPGASVKVSCKASGYTFTSYGISWVRQAPGQGLEWMGGIIPIFGTANYAQKFQGRVTITADESTSTAYMELSSLRSEDTAVYYCAKDLYDYVWGSYRPNWYFDLWGRGTLVTVSS

SEQ ID NO: 59 - DNA, 330 bp, synthetic construct
  source 1..330: mol_type = other DNA; note = CD137-VL-cys
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggcgctcactttcggctgcgggaccaaggtggagatcaaacga

SEQ ID NO: 60 - protein, 110 aa, synthetic construct
  source 1..110: note = CD137-VL-cys
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGCGTKVEIKR

SEQ ID NO: 61 - DNA, 363 bp, synthetic construct
  source 1..363: mol_type = other DNA; note = CD137-VH-cys
caggtgcaactacagcagtggggcgcaggactgttgaagccttcggagaccctgtccctcacttgcgctgtctatggtgggtccttcagtggttactactggagctggatacgccagtccccagagaagtgtctggagtggattggggaaatcaatcatggtggatacgtcacctacaatccgtccctggagagtcgagtcaccatatcagtagacacgtccaagaaccagttctccctgaagctgagctctgtgaccgccgcggacacggctgtatattactgtgcgagggactatggtccggggaattatgactggtacttcgatctctggggccgtggcaccctggtcactgtctcctcg

SEQ ID NO: 62 - protein, 121 aa, synthetic construct
  source 1..121: note = CD137-VH-cys
QVQLQQWGAGLLKPSETLSLTCAVYGGSFSGYYWSWIRQSPEKCLEWIGEINHGGYVTYNPSLESRVTISVDTSKNQFSLKLSSVTAADTAVYYCARDYGPGNYDWYFDLWGRGTLVTVSS

SEQ ID NO: 63 - protein, 18 aa, synthetic construct
  source 1..18: note = Linker-NKp46-scFv
VEGGSGGSGGSGGSGGVD

SEQ ID NO: 64 - DNA, 20 bp, synthetic construct
  source 1..20: mol_type = other DNA; note = Linker-NKG2D-scFv
ggggsggggsggggsggggs

SEQ ID NO: 65 - DNA, 54 bp, synthetic construct
  source 1..54: mol_type = other DNA; note = Linker-NKp46-scFv
gttgaaggtggatccggcggatcaggagggagtggtggcagtggaggtgttgac

SEQ ID NO: 66 - DNA, 54 bp, synthetic construct
  source 1..54: mol_type = other DNA; note = Linker-NKG2D-scFv
gggagtgcatccgccccaaagcttgaagaaggtgaattttcagaagcacgcgta

SEQ ID NO: 67 - protein, 133 aa, synthetic construct [sequence omitted]

SEQ ID NO: 68 - DNA, 399 bp, synthetic construct
  source 1..399: mol_type = other DNA; note = IL2
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactaact

SEQ ID NO: 69 - protein, 757 aa, synthetic construct
  source 1..757: note = CD19-CD137scFv-IL15
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSAQPAMAEIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRGGGGSGGGGSGGGGSGGGGSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASGAGGGGSGGGGSGGGGSITCPPPMSVEHADIWVKSYSLYSRERYICNSGFKRKAGTSSLTECVLNKATNVAHWTTPSLKCIRDPALVHQRPAPPGGGGSGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSRGGPEQKLISEEDLNSAVDHHHHHH

SEQ ID NO: 70 - DNA, 2271 bp, synthetic construct
  source 1..2271: mol_type = other DNA; note = CD19-CD137scFv-IL15
gaggtgcagctggtggagtctgggggaggcttggtaaagcctggggggtctcttaaactctcctgtgcagcctctggatatactttcactagctatgttatgcactgggtccgccaggctccagggaaggggctggagtggattggatatattaatccttacaatgatggtactaagtacaatgagaagttccagggcagagtcaccatttcctccgacaaatccattagcacagcctacatggagctgagcagcctgagatctgaggacacggccatgtattactgtgcaagagggacttattactacggtaccagggtatttgactactggggccaaggcactctcgtcaccgtctcctcggcctccactaagggacctagcgttttcccactggctccttcctctaagagcacttccggtggaacagccgctctcggatgcctggtcaaggattacttcccagaacccgtgactgtgagctggaatagcggcgcattgacctcaggggtccacacattcccagccgtacttcagagttccggcctgtatagcctgtcctctgtcgtgaccgttcctagctcaagcctcggtacacagacctacatctgcaacgtgaaccataagcccagcaacaccaaggtggacaagaaagtcgaacccaaatcctgcgacaaaacgcacacaggaggaggcggatctggtggaggaggatcaggaggaggcggatcggcccagccggccatggcggaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggcgctcactttcggcggagggaccaaggtggagatcaaacgaggtggtggtggttctggtggtggtggttctggcggcggcggctccggtggtggtggatcccaggtgcaactacagcagtggggcgcaggactgttgaagccttcggagaccctgtccctcacttgcgctgtctatggtggctccttcagtggttactactggagctggatacgccagtccccagagaaggggctggagtggattggggaaatcaatcatggtggatacgtcacctacaatccgtccctggagagtcgagtcaccatatcagtagacacgtccaagaaccagttctccctgaagctgagctctgtgaccgccgcggacacggctgtatattactgtgcgagggactatggtccggggaattatgactggtacttcgatctctggggccgtggcaccctggtcactgtctcctcggcctcgggggccggtggtggtggttctggcggcggcggctccggtggtggtggatccatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccacccggtggtggtggttctggtggtggtggttctggcggcggcggctccggtggtggtggatccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttctcgaggagggcccgaacaaaaactcatctcagaagaggatctgaatagcgccgtcgaccatcatcatcatcatcat

SEQ ID NO: 71 - protein, 757 aa, synthetic construct
  source 1..757: note = RTX-CD137scFvdss-IL15
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSAQPAMAEIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGCGTKVEIKRGGGGSGGGGSGGGGSGGGGSQVQLQQWGAGLLKPSETLSLTCAVYGGSFSGYYWSWIRQSPEKCLEWIGEINHGGYVTYNPSLESRVTISVDTSKNQFSLKLSSVTAADTAVYYCARDYGPGNYDWYFDLWGRGTLVTVSSASGAGGGGSGGGGSGGGGSITCPPPMSVEHADIWVKSYSLYSRERYICNSGFKRKAGTSSLTECVLNKATNVAHWTTPSLKCIRDPALVHQRPAPPGGGGSGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSRGGPEQKLISEEDLNSAVDHHHHHH

SEQ ID NO: 72 - DNA, 2271 bp, synthetic construct
  source 1..2271: mol_type = other DNA; note = RTX-CD137scFvdss-IL15
caggtacaactgcagcagcctggagccgagttggtgaagcctggcgccagcgtcaaaatgagctgcaaagcctcaggctacaccttcaccagctacaacatgcactgggtgaagcagacaccaggacgaggactggagtggatcggcgctatctaccctggcaacggagacacctcctacaaccagaagttcaaggggaaggcaacactgaccgctgacaagtccagcagcacagcctacatgcagctgagctccctgacttccgaggatagcgctgtgtactactgcgcaaggtccacctactacggaggcgactggtacttcaacgtgtggggcgccggaactaccgtgactgtgagcagcgcctccactaagggacctagcgttttcccactggctccttcctctaagagcacttccggtggaacagccgctctcggatgcctggtcaaggattacttcccagaacccgtgactgtgagctggaatagcggcgcattgacctcaggggtccacacattcccagccgtacttcagagttccggcctgtatagcctgtcctctgtcgtgaccgttcctagctcaagcctcggtacacagacctacatctgcaacgtgaaccataagcccagcaacaccaaggtggacaagaaagtcgaacccaaatcctgcgacaaaacgcacacaggaggaggcggatctggtggaggaggatcaggaggaggcggatcggcccagccggccatggcggaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggcgctcactttcggctgcgggaccaaggtggagatcaaacgaggtggtggtggttctggtggtggtggttctggcggcggcggctccggtggtggtggatcccaggtgcaactacagcagtggggcgcaggactgttgaagccttcggagaccctgtccctcacttgcgctgtctatggtgggtccttcagtggttactactggagctggatacgccagtccccagagaagtgtctggagtggattggggaaatcaatcatggtggatacgtcacctacaatccgtccctggagagtcgagtcaccatatcagtagacacgtccaagaaccagttctccctgaagctgagctctgtgaccgccgcggacacggctgtatattactgtgcgagggactatggtccggggaattatgactggtacttcgatctctggggccgtggcaccctggtcactgtctcctcggcctcgggggccggtggtggtggttctggcggcggcggctccggtggtggtggatccatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccacccggtggtggtggttctggtggtggtggttctggcggcggcggctccggtggtggtggatccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttctcgaggagggcccgaacaaaaactcatctcagaagaggatctgaatagcgccgtcgaccatcatcatcatcatcat

SEQ ID NO: 73 - protein, 653 aa, synthetic construct
  source 1..653: note = RTX-CD137scFv-IL2
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSAQPAMAEIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRGGGGSGGGGSGGGGSGGGGSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASGAGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 74 - DNA, 1959 bp, synthetic construct
  source 1..1959: mol_type = other DNA; note = RTX-CD137scFv-IL2
caggtacaactgcagcagcctggagccgagttggtgaagcctggcgccagcgtcaaaatgagctgcaaagcctcaggctacaccttcaccagctacaacatgcactgggtgaagcagacaccaggacgaggactggagtggatcggcgctatctaccctggcaacggagacacctcctacaaccagaagttcaaggggaaggcaacactgaccgctgacaagtccagcagcacagcctacatgcagctgagctccctgacttccgaggatagcgctgtgtactactgcgcaaggtccacctactacggaggcgactggtacttcaacgtgtggggcgccggaactaccgtgactgtgagcagcgcctccactaagggacctagcgttttcccactggctccttcctctaagagcacttccggtggaacagccgctctcggatgcctggtcaaggattacttcccagaacccgtgactgtgagctggaatagcggcgcattgacctcaggggtccacacattcccagccgtacttcagagttccggcctgtatagcctgtcctctgtcgtgaccgttcctagctcaagcctcggtacacagacctacatctgcaacgtgaaccataagcccagcaacaccaaggtggacaagaaagtcgaacccaaatcctgcgacaaaacgcacacaggaggaggcggatctggtggaggaggatcaggaggaggcggatcggcccagccggccatggcggaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggcgctcactttcggcggagggaccaaggtggagatcaaacgaggtggtggtggttctggtggtggtggttctggcggcggcggctccggtggtggtggatcccaggtgcaactacagcagtggggcgcaggactgttgaagccttcggagaccctgtccctcacttgcgctgtctatggtgggtccttcagtggttactactggagctggatacgccagtccccagagaaggggctggagtggattggggaaatcaatcatggtggatacgtcacctacaatccgtccctggagagtcgagtcaccatatcagtagacacgtccaagaaccagttctccctgaagctgagctctgtgaccgccgcggacacggctgtatattactgtgcgagggactatggtccggggaattatgactggtacttcgatctctggggccgtggcaccctggtcactgtctcctcggcctcgggggccggtggtggtggttctggcggcggcggctccggtggtggtggatccgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactaact

SEQ ID NO: 75 - protein, 753 aa, synthetic construct
  source 1..753: note = RTX-NKp46scFv-IL15
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSAQPAMAEVQLQESGPGLVKPSQSLSLTCTVTGYSITSDYAWNWIRQFPGNKLEWMGYITYSGSTSYNPSLESRISITRDTSTNQFFLQLNSVTTEDTATYYCARGGYYGSSWGVFAYWGQGTLVTVSAVEGGSGGSGGSGGSGGVDDIQMTQSPASLSASVGETVTITCRVSENIYSYLAWYQQKQGKSPQLLVYNAKTLAEGVPSRFSGSGSGTQFSLKINSLQPEDFGSYYCQHHYGTPWTFGGGTKLEIKASGAGGGGSGGGGSGGGGSITCPPPMSVEHADIWVKSYSLYSRERYICNSGFKRKAGTSSLTECVLNKATNVAHWTTPSLKCIRDPALVHQRPAPPGGGGSGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSRGGPEQKLISEEDLNSAVDHHHHHH

SEQ ID NO: 76 - DNA, 2259 bp, synthetic construct
  source 1..2259: mol_type = other DNA; note = RTX-NKp46scFv-IL15
caggtacaactgcagcagcctggagccgagttggtgaagcctggcgccagcgtcaaaatgagctgcaaagcctcaggctacaccttcaccagctacaacatgcactgggtgaagcagacaccaggacgaggactggagtggatcggcgctatctaccctggcaacggagacacctcctacaaccagaagttcaaggggaaggcaacactgaccgctgacaagtccagcagcacagcctacatgcagctgagctccctgacttccgaggatagcgctgtgtactactgcgcaaggtccacctactacggaggcgactggtacttcaacgtgtggggcgccggaactaccgtgactgtgagcagcgcctccactaagggacctagcgttttcccactggctccttcctctaagagcacttccggtggaacagccgctctcggatgcctggtcaaggattacttcccagaacccgtgactgtgagctggaatagcggcgcattgacctcaggggtccacacattcccagccgtacttcagagttccggcctgtatagcctgtcctctgtcgtgaccgttcctagctcaagcctcggtacacagacctacatctgcaacgtgaaccataagcccagcaacaccaaggtggacaagaaagtcgaacccaaatcctgcgacaaaacgcacacaggaggaggcggatctggtggaggaggatcaggaggaggcggatcggcccagccggccatggcggaggtacagttgcaggagagtgggcctggactggtcaaaccctcccaatctctgagcttgacatgcacagtcacaggctacagcatcacctccgactacgcttggaattggattcgacagtttcccggcaacaagctggaatggatgggctacatcacctatagtggtagcacttcctataatccctcacttgagagccggatttccatcactagggatacgagcaccaaccagttcttcctgcagctgaatagcgtcaccaccgaagatactgccacctattactgcgcaagaggcggttactatggcagttcatggggtgtattcgcctattggggacaggggacacttgtgacagtgtctgctgttgaaggtggatccggcggatcaggagggagtggtggcagtggaggtgttgacgacattcagatgacccaatcccctgcttctctctcagcctctgtgggagagactgtgaccataacctgtcgtgttagcgagaacatctactcctatctcgcctggtatcagcagaaacaggggaaatccccacaactgctcgtgtacaatgccaagactctggcagaaggagtgccaagccgcttttccgggtctgggtctgggacacagttctcactgaagatcaactctttgcaacctgaggattttggctcttactactgtcagcatcactatggcacaccatggacgtttggtggcggcactaagctggagattaaggcctcgggggccggtggtggtggttctggcggcggcggctccggtggtggtggatccatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccacccggtggtggtggttctggtggtggtggttctggcggcggcggctccggtggtggtggatccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttctcgaggagggcccgaacaaaaactcatctcagaagaggatctgaatagcgccgtcgaccatcatcatcatcatcat

SEQ ID NO: 77 - protein, 759 aa, synthetic construct
  source 1..759: note = RTX-NKG2DscFv-IL15
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSAQPAMAQVQLVQSGAEVKKPGASVKVSCKASGYTFTSYGISWVRQAPGQGLEWMGGIIPIFGTANYAQKFQGRVTITADESTSTAYMELSSLRSEDTAVYYCAKDLYDYVWGSYRPNWYFDLWGRGTLVTVSSGSASAPKLEEGEFSEARVSSELTQDPAVSVALGQTVRITCQGDSLRGYYASWYQQKPGQAPVLVTYGNNKRPSGIPDRFSGSSSGNTASLTITGTQAEDEADYYCNSRDNNGNFVIFGGGTKLTVLASGAGGGGSGGGGSGGGGSITCPPPMSVEHADIWVKSYSLYSRERYICNSGFKRKAGTSSLTECVLNKATNVAHWTTPSLKCIRDPALVHQRPAPPGGGGSGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSRGGPEQKLISEEDLNSAVDHHHHHH

SEQ ID NO: 78 - DNA, 2277 bp, synthetic construct
  source 1..2277: mol_type = other DNA; note = RTX-NKG2DscFv-IL15
caggtacaactgcagcagcctggagccgagttggtgaagcctggcgccagcgtcaaaatgagctgcaaagcctcaggctacaccttcaccagctacaacatgcactgggtgaagcagacaccaggacgaggactggagtggatcggcgctatctaccctggcaacggagacacctcctacaaccagaagttcaaggggaaggcaacactgaccgctgacaagtccagcagcacagcctacatgcagctgagctccctgacttccgaggatagcgctgtgtactactgcgcaaggtccacctactacggaggcgactggtacttcaacgtgtggggcgccggaactaccgtgactgtgagcagcgcctccactaagggacctagcgttttcccactggctccttcctctaagagcacttccggtggaacagccgctctcggatgcctggtcaaggattacttcccagaacccgtgactgtgagctggaatagcggcgcattgacctcaggggtccacacattcccagccgtacttcagagttccggcctgtatagcctgtcctctgtcgtgaccgttcctagctcaagcctcggtacacagacctacatctgcaacgtgaaccataagcccagcaacaccaaggtggacaagaaagtcgaacccaaatcctgcgacaaaacgcacacaggaggaggcggatctggtggaggaggatcaggaggaggcggatcggcccagccggccatggcgcaggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgaaagatctctatgattacgtttgggggagttatcgccctaactggtacttcgatctctggggccgtggcaccctggtcactgtctcctcagggagtgcatccgccccaaagcttgaagaaggtgaattttcagaagcacgcgtatcttctgagctgactcaggaccctgctgtgtctgtggccttgggacagacagtcaggatcacatgccaaggagacagcctcagaggctattatgcaagctggtaccagcagaagccaggacaggcccctgtacttgtcacctatggtaacaacaagcggccctcaggaatcccagaccgattctctggctccagttcaggaaacacagcttccttgaccatcactgggactcaggcggaagatgaggctgactactattgtaactcccgggacaacaatggtaacttcgttatattcggcggagggaccaagctgaccgtcctggcctcgggggccggtggtggtggttctggcggcggcggctccggtggtggtggatccatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccacccggtggtggtggttctggtggtggtggttctggcggcggcggctccggtggtggtggatccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttctcgaggagggcccgaacaaaaactcatctcagaagaggatctgaatagcgccgtcgaccatcatcatcatcatcat

SEQ ID NO: 79 - protein, 663 aa, synthetic construct
  source 1..663: note = IL15-RTXscFv-41BB-ligand
ITCPPPMSVEHADIWVKSYSLYSRERYICNSGFKRKAGTSSLTECVLNKATNVAHWTTPSLKCIRDPALVHQRPAPPGGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSGGGSSGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSSGGGGSGGGGSGGSAQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKAAAHHHHHHGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 80 - DNA, 1989 bp, synthetic construct
  source 1..1989: mol_type = other DNA; note = IL15-RTXscFv-41BB-ligand
atcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccacccggtggtggttctggtggtggtggttctggcggcggctccggtggtggtggatccctgcagaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctggaggcggatctggaggcggatcgtcaggaggaggatcacaggtacaactgcagcagcctggagccgagttggtgaagcctggcgccagcgtcaaaatgagctgcaaagcctcaggctacaccttcaccagctacaacatgcactgggtgaagcagacaccaggacgaggactggagtggatcggcgctatctaccctggcaacggagacacctcctacaaccagaagttcaaggggaaggcaacactgaccgctgacaagtccagcagcacagcctacatgcagctgagctccctgacttccgaggatagcgctgtgtactactgcgcaaggtccacctactacggaggcgactggtacttcaacgtgtggggcgccggaactaccgtgactgtgagcagcggtggtggtggttctggtggtggtggttctggtggttctgcccaaatcgtcctcagccagtctccagccatcctctccgcatctcctggcgagaaggtgaccatgacctgcagagcttccagctccgtcagctacatccactggttccagcagaagcctggaagcagcccaaagccctggatctacgccaccagcaacctggccagcggagtcccagtgaggttctcagggagcggaagtggcacctcctacagcctgaccatcagcagggtggaggctgaagatgcagccacctactactgccagcagtggacctccaatcctcccacctttggtggaggaaccaagcttgagatcaaggccgccgcccatcatcatcatcatcatggtggtggtggttctcgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcatctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaa